(19) United States
(12) Patent Application Publication
(10) Pub. No.: US 2026/0209749 A1
(43) Pub. Date: Jul. 23, 2026

(54) ELECTROCHEMICAL SYNTHESIS WITH REDOX STABLE NUCLEOTIDES

(71) Applicant: ATLAS DATA STORAGE, INC., Chicago, IL (US)

(72) Inventors: Tiandi Wu, South San Francisco, CA (US); Jeremy LACKEY, Foster City, CA (US); Yan You LIN, South San Francisco, CA (US); Stefan PITSCH, South San Francisco, CA (US)

(21) Appl. No.: 19/512,208

(22) PCT Filed: Feb. 1, 2024
(86) PCT No.: PCT/US2024/013992

§ 371 Date: Feb. 18, 2026

Related U.S. Application Data
(60) Provisional application No. 63/482,653, filed on Feb. 1, 2023.

(51) Int. Cl. C12N 15/10

(52) U.S. Cl. CPC C12N 15/1065

(57) ABSTRACT

Provided herein are compositions, devices, systems and methods for constructing and storing polynucleotides encoding information with redox resistant bases. The compositions, devices, systems, and methods described herein provide for storage or synthesis of a library comprising a plurality of polynucleotides with one or more redox resistance bases. Further provided herein are methods to increase DNA synthesis yield and fidelity.

### CROSS-REFERENCE TO RELATED APPLICATION

This application claims priority to U.S. Provisional Patent Application No. 63/482,653, filed on Feb. 1, 2023, which is hereby incorporated by reference in its entirety. All publications, patents, and patent applications mentioned in this specification are herein incorporated by reference to the same extent as if each individual publication, patent, or patent application was specifically and individually indicated to be incorporated by reference.

### BACKGROUND

Biomolecules (e.g., nucleic acids) have applications in research, medicine, and information storage. However, there is a need for high-density, scalable, automated, highly accurate and highly efficient systems for generating biomolecules.

### BRIEF SUMMARY

Provided herein is a library comprising a plurality of polynucleotides, wherein the plurality of polynucleotides comprises a redox resistant base, and wherein the library encodes an item of information. In some instances, at least 10% of bases in the plurality of polynucleotides comprise the redox resistant base. In some instances, at least one of four canonical bases is replaced with a redox resistant base. In some instances, the redox resistant base is a non-canonical base. In some instances, the non-canonical base can pair with a canonical base. In some instances, the redox resistant base has an oxidation potential larger than that of deoxyguanosine. In some instances, a ratio of redox resistant bases comprising the non-canonical base to canonical bases in the plurality of polynucleotides is about 1:1 to about 1:9. In some instances, the plurality of polynucleotides comprises at least one, two, or three different canonical bases. In some instances, the plurality of polynucleotides comprise adenosine, thymidine, cytidine, or any combination thereof. In some instances, the non-canonical base comprises diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, or 2,6-diaminopurine. In some instances, the non-canonical base comprises inosine. In some instances, the plurality of polynucleotides comprises 50 to 300 bases in length. In some instances, the item of information comprises text information, audio information, visual information, or any combination thereof. In some instances, each of the plurality of polynucleotides comprises at least one data block and at least one non-data block. In some instances, the at least one data block comprises a portion of the item of information. In some instances, the at least one non-data block comprises metadata related to the item of information. In some instances, the metadata comprises an index, data type, data size, data format, encryption codec, date of synthesis, date of last access, dates of previous handling, owner information, manufacture information, storage mechanism, or any combination thereof. In some instances, the item of information is stored in the library with at least 10% redundancy. In some instances, the plurality of polynucleotides comprises 1000 to 500,000 polynucleotides. In some instances, the library comprises at least one adaptor sequence. In some instances, the at least one adaptor is configured to bind to a flow cell. In some instances, the library comprises at least one barcode.

Further provided herein is a method of storing an item of information in a plurality of polynucleotides, comprising: converting a first string of symbols encoding an item of information to a second string of symbols, wherein the second string of symbols comprises sequences of a plurality of polynucleotides in a library provided herein. In some instance, the plurality of polynucleotides in the library comprises a redox resistant base. In some instances, the method further comprises constructing the library comprising the plurality of polynucleotides. In some instances, the method further comprises storing the library comprising the plurality of polynucleotides. In some instances, converting the item of information comprises: (a) generating a codec comprising one or more rules; and (b) applying the codec to the first string of symbols to generate the second string of symbols. In some instances, the one or more rules comprises an error correction scheme, a codebook, a sequence constraint, or any combination thereof. In some instances, the sequence constraint comprises one or more constraints related to length, inosine content, guanosine content, guanosine cytosine content, repeats of one or more bases, or any combination thereof. In some instances, the second string of symbols comprises at least one data block and at least one non-data block. In some instances, at least one data block comprises a portion of the item of information. In some instances, the at least one non-data block comprises metadata related to the item of information. In some instances, the metadata comprises an index, data type, data size, data format, encryption codec, date of synthesis, date of last access, dates of previous handling, owner information, manufacture information, storage mechanism, or any combination thereof. In some instances, constructing comprises synthesizing the plurality of polynucleotides.

Further provided herein is a method for synthesizing a library provided herein. In some instance, the plurality of polynucleotides in the library comprises a redox resistant base. In some instances, synthesizing comprises electrochemical deblocking using electrochemical acid generation. In some instances, electrochemical acid generation comprises contacting a protected polynucleotide with a composition comprising one or more redox compounds. In some instances, synthesizing comprises: (a) contacting a nucleoside attached to a solid support with a protected nucleoside, wherein the protected nucleoside is configured to form a covalent bond with the nucleoside to generate a protected polynucleotide; (b) contacting the protected polynucleotide with a composition comprising one or more redox compounds, and (c) applying a voltage to a solvent in fluid communication with the protected polynucleotide, wherein the voltage results in deprotection of the terminal nucleoside of the protected polynucleotide. In some instances, the composition further comprises an organic salt and at least one solvent. In some instances, the one or more redox compounds comprises a substituted or unsubstituted quinone. In some instances, the one or more redox compounds comprises a mixture of quinone and benzoquinone. In some instances, the organic salt comprises a tetraalkylammonium cation. In some instances, the organic salt comprises a hexafluorophosphate anion. In some instances, the organic salt is tetrabutylammonium hexafluorophosphate. In some instances, the at least one solvent is acetonitrile, methanol, ethanol, dichloromethane, chloroform, 1,2-dichloromethane, dimethylformamide, ethylene glycol, propylene carbonate, or a mixture thereof. In some instances, a concentration of the one or more redox compounds is 0.1-2M. In some instances, concentration of the organic salt is 10-50 mM. In some instances, the voltage is less than 2 volts. In some instances, the voltage is 0.1-2 volts. In some instances, the voltage is applied for 0.001-5000 seconds. In some instances, the voltage is applied for 0.001-5 seconds. In some instances, the voltage is applied in one or more pulses. In some instances, the time between pulses is 0-500 milliseconds. In some instances, the protected polynucleotide comprises an acid-cleavable protecting group. In some instances, the voltage generates acid.

Further provided herein a method for retrieving an item of information. In some instances, the item of information is stored in a library comprising a plurality of polynucleotides provided herein. In some instance, the plurality of polynucleotides in the library comprises a redox resistant base. In some instances, retrieving the item of information comprises: (a) sequencing the library comprising the plurality of polynucleotides to obtain a readout; and (b) converting readout into the item of information. In some instances, retrieving the item of information further comprises amplifying the plurality of polynucleotides. In some instances, the item of information is retrieved with at least 99% accuracy. In some instances, converting the readout into the item of information comprises: (a) applying the codec or a portion thereof to the readout comprising a third string of symbols to generate a fourth string of symbols; and (b) assembling the fourth string of symbols to retrieve the item of information. In some instances, the second string of symbols and the third string of symbols comprise nucleic acid sequences. In some instances, the second string of symbols and the third string of symbols are at least 99% identical. In some instances, the first string of symbols and the fourth string of symbols are at least 99% identical.

Further provided herein is a method for increasing fidelity of DNA encoding an item of information, comprising replacing one or more bases of a DNA sequence with a redox resistant base.

Further provided herein is a method for storing DNA encoding an item of information, comprising: (a) receiving a DNA sequence encoding an item of information; (b) replacing one or more bases of the DNA sequence with a redox resistant base; and (c) storing the DNA sequence with the redox resistant base. In some instances, replacing the one or more bases comprises replacing at least 10% of bases in the DNA sequence with the lowest oxidation potential.

Further provided herein is a device for polynucleotide synthesis comprising: a surface comprising a plurality of loci configured for polynucleotide synthesis of the library provided herein; and a plurality of vias or routing configured for addressable control of the plurality of loci, wherein the area of each loci is 50-500 nm. In some instances, the loci comprises a pitch distance of no more than 1000 nm. In some instances, the device comprises at least 10 loci per square micron. In some instances, the device is integrated into a CMOS. In some instances, the device further comprises a fluidics interface.

Further provided herein is a device for storing an item of information in a plurality of polynucleotides, comprising: one or more compartments, wherein each compartment comprises: a library provided herein or a portion thereof, wherein the library encodes the item of information, or a portion thereof; and a medium for storing the library or the portion thereof. In some instances, the one or more compartments are in communication. In some instances, the one or more compartments are not in communication. In some instances, the one or more compartments are independently accessible. In some instances, each of the one or more compartments are independently accessible via a robotic system. In some instances, the medium comprises a solid, a liquid, a gas, or any combination thereof. In some instances, a medium comprises a salt solution at a molar ratio of less than 20:1 salt cation to phosphate groups in the DNA. In some instances, the salt solution is dried to create a dried product. In some instances, the device further comprises a solid support comprising a surface. In some instances, the device further comprises a plurality of structures located on the surface, wherein the plurality of polynucleotide are extended from the plurality of structures.

Further provided herein is a system for storing an item of information, comprising: (a) a computing system comprising at least one processor and instructions executable by the at least one processor to perform one or more operations, the one or more operations comprising: converting a first string of symbols to a second string of symbols, wherein the second string of symbols comprises a DNA sequence with a redox resistant base; and (b) a material deposition system in communication with the computing system, comprising: (i) a substrate for constructing the DNA sequence; and (ii) a deposition unit for depositing one or more building blocks, reagents, or both for constructing the DNA sequence.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B illustrates deoxyguanosine (FIG. 1A) and its overoxidation (FIG. 1B), according to some embodiments.

FIG. 2 illustrates the electrochemical deblocking process, according to some embodiments.

FIG. 3 illustrates the structure of deoxyguanosine (dG) and deoxyinosine (dI), according to some embodiments.

FIG. 4 illustrates LCMS chromatograms from polynucleotides comprising dG (bottom) and polynucleotides that have replaced dG with dI (top), according to some embodiments.

FIG. 5 illustrates an exemplary workflow for storing information in polynucleotides, according to some embodiments.

FIG. 6 illustrates an exemplary workflow for retrieving information stored in polynucleotides, according to some embodiments.

FIGS. 7A-7F illustrates non-limiting examples of high-density devices and cross-sectional views, according to some embodiments. FIG. 7A illustrates a cross-section view of a high-density device for polynucleotide synthesis. Two exemplary addressable device arrays are shown for clarity only. FIG. 7B illustrates a top view of a high-density device for polynucleotide synthesis. Nine exemplary addressable device arrays are shown for clarity only. FIG. 7C illustrates a top view of a high-density device for polynucleotide synthesis. Four exemplary addressable device arrays are shown for clarity only. FIG. 7D illustrates a cross-section view of a high-density device for polynucleotide synthesis. Two exemplary device arrays are shown for clarity only. FIG. 7E illustrates a top view of a high-density device for polynucleotide synthesis. Sixteen exemplary addressable device arrays are shown for clarity only. FIG. 7F depicts a schematic of a CMOS-integrated device array.

FIGS. 8A-8D illustrate non-limiting examples of solid supports and their storage, according to some embodiments. FIG. 8A is an example of rack-style instrument. Such instruments may comprise hundreds or thousands of solid support arrays according to some embodiments. FIG. 8B is a front side of an example of a solid support array according to some embodiments. Such arrays in some instances may comprise thousands or millions of polynucleotide synthesis devices as described herein. FIG. 8C is a back side of an example of a solid support array according to some embodiments. FIG. 8D is a schema of solid support comprising an active area and fluidics interface according to some embodiments.

FIG. 9 illustrates an example of a computer system according to some embodiments.

FIG. 10 is a block diagram illustrating architecture of a computer system according to some embodiments.

FIG. 11 is a diagram demonstrating a network configured to incorporate a plurality of computer systems, a plurality of cell phones and personal data assistants, and Network Attached Storage (NAS) according to some embodiments.

FIG. 12 is a block diagram of a multiprocessor computer system using a shared virtual address memory space according to some embodiments.

FIG. 13 shows cyclic voltammetry data of dG and dI phosphoramidite, including the oxidation potential of substrates, according to some embodiments.

### DETAILED DESCRIPTION

There is a need for larger capacity storage systems as the amount of information generated and stored is increasing exponentially. A biomolecule such as a DNA molecule provides a suitable host for information storage in-part due to its stability over time and capacity for four bit information coding, as opposed to traditional binary information coding. Provided herein are methods to increase DNA synthesis yield and fidelity, as well as decreased error rates using redox resistant bases.

### Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects are described herein primarily with regard to the preparation of nucleic acids molecules, the sequence of the polynucleotide being known and chosen before the synthesis or assembly of the nucleic acid molecules.

As used herein, the term “symbol,” generally refers to a representation of a unit of digital information. Digital information may be divided or translated into one or more symbols. In an example, a symbol may be a bit and the bit may have a numerical value. In some examples, a symbol may have a value of ‘0’ or ‘1’. In some examples, digital information may be represented as a sequence of symbols or a string of symbols. In some examples, the sequence of symbols or the string of symbols may comprise binary data.

Provided herein are methods and compositions for production of synthetic (e.g. de novo synthesized or chemically synthesized) polynucleotides. Polynucleotides may also be referred to as oligonucleotides or oligos. Polynucleotide sequences described herein may be, unless stated otherwise, comprise DNA or RNA.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(G)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R, —C(O)OR^{a}, —C(O)N(R^{a})₂, —N(R^{a})C(O)OR, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hckel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R⁵—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —RL—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜRa (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical are optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” or “heterocycloalkyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R⁵—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{a}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hickel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

Provided herein are methods and compositions for production of synthetic (e.g. de novo synthesized, enzymatically synthesized, chemically synthesized) biomolecules. In some instances, biomolecules are synthesized in a template-independent manner. In some instances, biomolecules comprise polynucleotides. Polynucleotides may also be referred to as oligonucleotides or oligos. Polynucleotide sequences described herein may be, unless stated otherwise, comprise DNA or RNA. In some instances, biomolecules comprise polymers which comprise two or more monomers. Biomolecules in some instances refer to polymers such as nucleic acids (e.g., DNA, RNA), carbohydrates (e.g., sugars), peptides/proteins, lipids, fatty acids, terpenes, peptoids, or mixture thereof. In some instances, biomolecules may be synthesized in an iterative fashion using methods well-known in the art (with or without protecting groups). In some instances, biomolecules may be synthesized in an iterative fashion from monomers, dimers, trimers, or other appropriate building block.

Provided herein are libraries comprising a plurality of polynucleotides. In some instances, the plurality of polynucleotides comprises a redox resistant base. In some instances, the library encodes an item of information.

Also provided herein are methods of storing an item of information in a plurality of polynucleotides. In some instances, the methods comprise converting a first string of symbols encoding an item of information to a second string of symbols. In some instances, the second string of symbols comprises sequences of a plurality of polynucleotides in a library provided herein. In some instances, converting the item of information comprises one or more of: (a) generating a codec comprising one or more rules; and (b) applying the codec to the first string of symbols to generate the second string of symbols.

Also provided herein are methods for constructing the library comprising the plurality of polynucleotides. In some instances, constructing comprises synthesizing the plurality of polynucleotides. In some instances, synthesizing comprises electrochemical deblocking using electrochemical acid generation. In some instances, electrochemical acid generation comprises contacting a protected polynucleotide with a composition comprising one or more redox compounds. In some instances, synthesizing comprises one or more of: (a) contacting a nucleoside attached to a solid support with a protected nucleoside; (b) contacting the protected polynucleotide with a composition comprising one or more redox compounds, and (c) applying a voltage to a solvent in fluid communication with the protected polynucleotide. In some instances, the protected nucleoside is configured to form a covalent bond with the nucleoside to generate a protected polynucleotide. In some instances, the voltage results in deprotection of the terminal nucleoside of the protected polynucleotide.

Further provided herein are methods for storing the library comprising the plurality of polynucleotides. In some instances, the plurality of polynucleotides are stored using the systems and methods provided herein.

Further provided herein are methods for retrieving an item of information. In some instances, the item of information is stored in a library comprising a plurality of polynucleotides provided herein. In some instances, retrieving the item of information comprises one or more of: (a) sequencing the library comprising the plurality of polynucleotides to obtain a readout; and (b) converting readout into the item of information. In some instances, retrieving the item of information further comprises amplifying the plurality of polynucleotides.

Further provided herein are methods for increasing fidelity of DNA encoding an item of information, comprising replacing one or more bases of a DNA sequence with a redox resistant base.

Further provided herein are methods for storing DNA encoding an item of information. In some instances, the methods comprise one or more of: (a) receiving a DNA sequence encoding an item of information; (b) replacing one or more bases of the DNA sequence with a redox resistant base; and (c) storing the DNA sequence with the redox resistant base.

Further provided herein are devices for polynucleotide synthesis. In some intances, the device can comprise one or more of: a surface comprising a plurality of loci configured for polynucleotide synthesis of the library provided herein; and a plurality of vias or routing configured for addressable control of the plurality of loci. In some instances, the area of each loci is 50-500 nm.

Further provided herein are devices for storing an item of information in a plurality of polynucleotides. In some instances, the device comprises one or more of: one or more compartments, and a medium for storing the library or the portion thereof. In some instances, each compartment comprises: a library provided herein or a portion thereof. In some instances, the library encodes the item of information, or a portion thereof.

Further provided herein are systems for storing an item of information. In some instances, a system can comprise one or more of: (a) a computing system comprising at least one processor and instructions executable by the at least one processor to perform one or more operations; and (b) a material deposition system in communication with the computing system. In some instances, the one or more operations comprises converting a first string of symbols to a second string of symbols. In some instances, the second string of symbols comprises a DNA sequence with a redox resistant base. In some instances, the material deposition system comprises one or more of: (i) a substrate for constructing the DNA sequence; and (ii) a deposition unit for depositing one or more building blocks, reagents, or both for constructing the DNA sequence.

### Redox Resistant Nucleic Acid Libraries

Provided herein are device, methods, compositions, and systems for storage or synthesis of a library comprising a plurality of polynucleotides with one or more redox resistance bases. The use of a redox resistant base in a plurality of polynucleotides can increase the quality of polynucleotides in synthesis or storage. For example, a redox resistant base is less susceptible to oxidative damage that can occur during synthesis or storage. In some instances, synthesis or storage of polynucleotides comprising a redox resistant base have an increased yield compared to polynucleotides without the redox resistant base. In some instances, synthesis or storage of polynucleotides comprising a redox resistant base have a lower error rate compared to polynucleotides without the redox resistant base. In some instances, synthesis or storage of polynucleotides comprising a redox resistant base have increased fidelity compared to polynucleotides without the redox resistant base.

In some instances, a library encodes information, such as an item of information or a plurality of items of information. An item of information may be text information, audio information, visual information, or any combination thereof, such as those described herein. In some instances, an item of information does not comprise genomic information. Each of the plurality of polynucleotides may comprise a data block comprising the item of information and a non-data block comprising metadata of the item of information. In some instances, each of the plurality of polynucleotides comprises an adapter sequence for capture or amplification of the polynucleotide. In some instances, the adapter sequence is configured to bind to a flow cell.

The library can comprise about 1000 to 500,000 polynucleotides. Each of the plurality of polynucleotides can comprise about 50 to 300 bases in length. In some instances, each of the plurality of polynucleotides comprises at least one canonical base and at least one non-canonical base. In some instances, each of the plurality of polynucleotides comprises a redox resistant base. The canonical base may be a natural base, such as adenosine, thymidine, cytidine, or guanosine. In some instances, the non-canonical base is a redox resistant base.

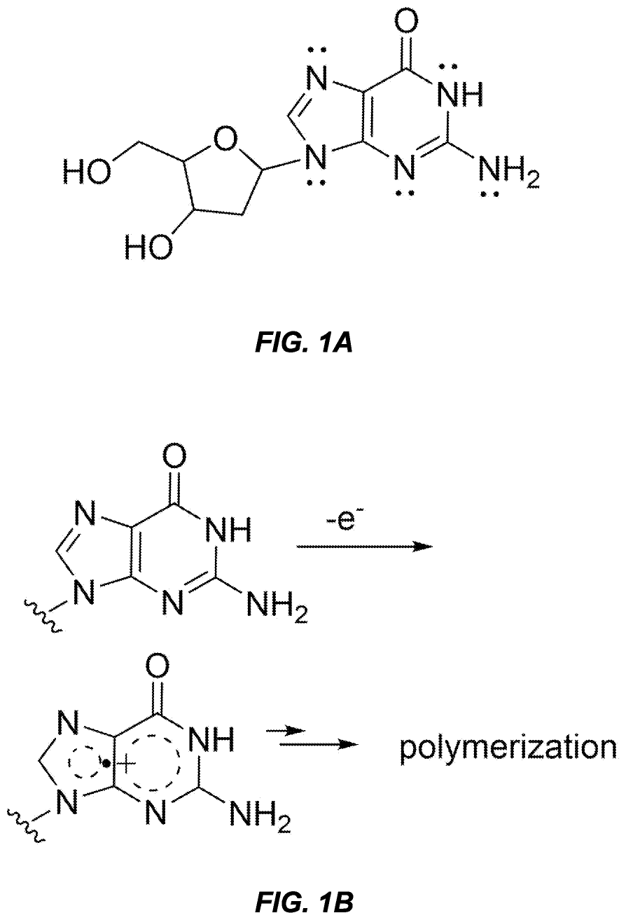

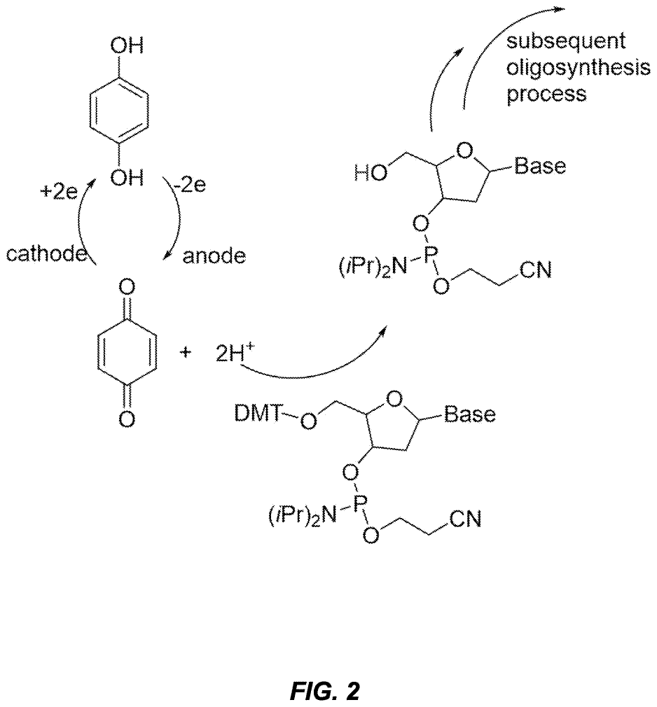

The quality of a polynucleotide may be reduced due to oxidation of a nucleobase during synthesis or storage. For example, deoxyguanosine (e.g., FIG. 1A) contains 2-amino substituted purine skeleton and is the most electron-rich nucleobase among the four canonical nucleobases (A, T, C, and G). Such a structure can be affected during electrochemical anodic process during synthesis of a polynucleotide (e.g., FIG. 2). In some instances, the structure can be affected by oxidation during storage of a polynucleotide. In some instances, overoxidation of guanosine occurs during synthesis or storage (e.g., FIG. 1B), thus yielding high error rates, lower yields, or both of the polynucleotides. Additionally, polynucleotides synthesized on a surface of a device or chip can be susceptible to oxidation due to proximity to an anode, for example, during electrochemical deblocking.

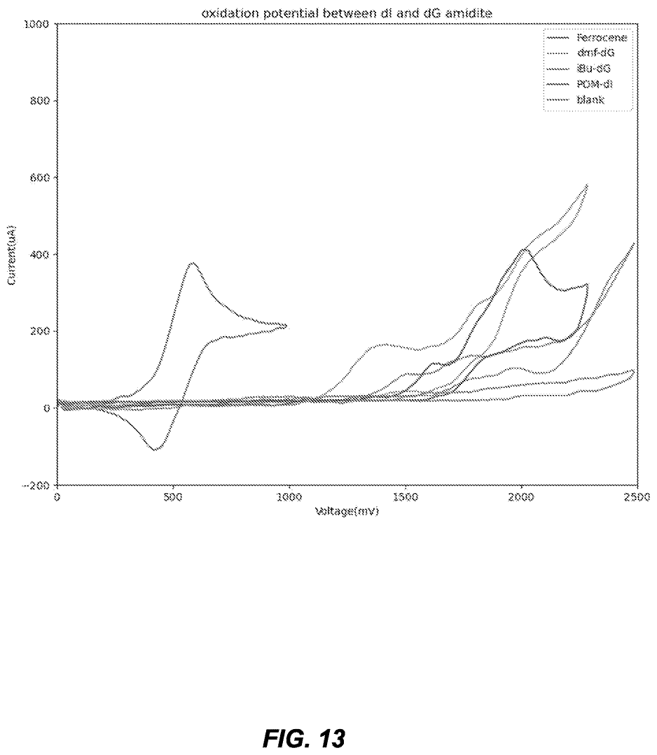

The quality of a polynucleotide can be increased by reducing oxidative damage that can occur to a polynucleotide during synthesis or storage. Oxidative damage may be reduced during synthesis by decreasing a voltage that is used for electrochemical deblocking. Alternatively, oxidative damage may be reduced by using bases that have a higher oxidation potential. In some instances, oxidative damage is reduced in a polynucleotide by employing a redox resistant base. A redox resistant base can be resistant to oxidation. In some instances, a redox resistant base has an oxidation potential larger than that of deoxyguanosine (dG) phosphoramidite (e.g., FIG. 13). In some instances, a redox resistant base in a non-canonical base (e.g., not a natural base, such as A, T, C, or G). In some instances, a redox resistant base is an unnatural base, such as those described herein.

In some instances, the non-canonical base (unnatural base) comprises any one of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifluoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N4-ethylcytosine, N-2 substituted purines, N-6 substituted purines, 0-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′: 4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methyl aminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine and those in which the purine or pyrimidine base is replaced with a heterocycle.

In some instances, the non-canonical base comprises any one of diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, or 2,6-diaminopurine.

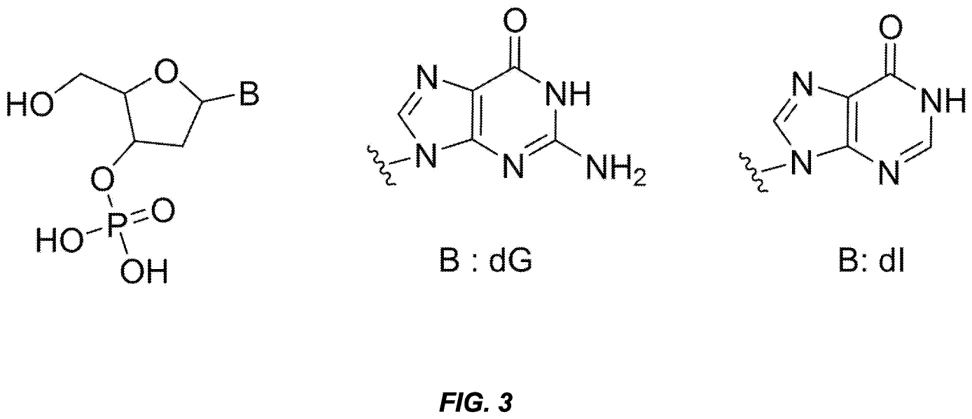

In some instances, the quality of a polynucleotide is increased by replacing all or a portion of guanosine with a redox resistant base (e.g., non-canonical base) that is less susceptible to oxidation. In some instances, the redox resistant base is inosine. In some instances, deoxyinosine (dI) phosphoramidite is used as a substitute for a canonical base that is most susceptible to oxidation, such as deoxyguanosine (dG) in electrochemical synthesis of a polynucleotide or a library comprising a plurality of polynucleotides. For example, dI shares the same purine backbone with dG but without 2-amino moiety (as shown in FIG. 3), and further has a lower oxidation potential than dG. Additionally, incorporation of dI results in amplification by DNA polymerases, as shown, for example in Kobayashi, et. al., 2004, “Analyses of PCR Products Using DNA Templates Containing A Consecutive Deoxyinosine Sequence,” Nucleic Acids Symp. Ser. 225-226, which is incorporated herein by reference in its entirety. In some instances, the use of dI in electrochemical synthesis of DNA is motivated due to similar characteristics between the two nucleobases, dI and dG, with favorability of a lower redox potential of dI. In some instances, replacing dI with dG results in less oxidative damage to the nucleobase during a redox process at the anode during synthesis, thus increasing quality of polynucleotides synthesized. In some examples, replacing dI with dG results in less oxidative damage to polynucleotides by at least 5%, 10%, 15%, 20%, 25%, 30%, 35%, 40%, 45%, 50%, 55%, 60%, 65%, 70%, 75%, 80%, 85%, 90%, 95%, 100%. In some instances, replacing dG with dI results in an increase in yield, decrease in error rate, or both. In some instances, replacing dG with dI results in an increase in yield in polynucleotides by at least 5%, 10%, 15%, 20%, 25%, 30%, 35%, 40%, 45%, 50%, 55%, 60%, 65%, 70%, 75%, 80%, 85%, 90%, 95%, 100%, 120%, 150%, or 200% or more. In some instances, replacing dG with dI results in a decrease in error rate in polynucleotides by at least 5%, 10%, 15%, 20%, 25%, 30%, 35%, 40%, 45%, 50%, 55%, 60%, 65%, 70%, 75%, 80%, 85%, 90%, 95%, or 100%.

Provided herein are libraries comprising redox resistant bases. Libraries comprising redox resistant bases can have higher quality, higher yields, lower error rates, less oxidative damage, or any combination thereof, compared to libraires without redox resistant bases. In some instances, at least 5%, 10%, 15%, 20%, 25%, 30%, 35%, 40%, 45%, 50%, 55%, 60%, 65%, 70%, or 75% of bases in a library comprising a plurality of polynucleotides are redox resistant bases. In some instances, all of the redox resistant bases are the same base (e.g., inosine). In some instances, the redox resistant bases are different bases. In some instances, at least one of the four canonical bases (e.g., A, T, C, and G) is replaced with a non-canonical base that is a redox resistant base (e.g., replacing G with I). In some instances, the ratio of non-canonical bases to canonical bases in a library comprising a plurality of polynucleotides is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or about 1:10. In some instances, the library comprises at least one, two, or three canonical bases. In some instances, the library comprises all four canonical bases. In some instances, the library comprises at least one, two, or three non-canonical bases, such as those provided herein. In some instances, the library comprises inosine.

In some instances, a library comprises about 1000 to 500,000 polynucleotides. In some instances, a library comprises 1,000 to 5,000, 1,000 to 10,000, 1,000 to 20,000, 1,000 to 50,000, 1,000 to 100,000, 1,000 to 200,000, 1,000 to 500,000, 5,000 to 10,000, 5,000 to 20,000, 5,000 to 50,000, 5,000 to 100,000, 5,000 to 200,000, 5,000 to 500,000, 10,000 to 20,000, 10,000 to 50,000, 10,000 to 100,000, 10,000 to 200,000, 10,000 to 500,000, 20,000 to 50,000, 20,000 to 100,000, 20,000 to 200,000, 20,000 to 500,000, 50,000 to 100,000, 50,000 to 200,000, 50,000 to 500,000, 100,000 to 200,000, 100,000 to 500,000, or 200,000 to 500,000 polynucleotides. In some instances, a library comprises 1,000, 5,000, 10,000, 20,000, 50,000, 100,000, 200,000, or 500,000 polynucleotides. In some instances, a library comprises at least 1,000, 5,000, 10,000, 20,000, 50,000, 100,000, or 200,000 polynucleotides. In some instances, a library comprises at most 5,000, 10,000, 20,000, 50,000, 100,000, 200,000, or 500,000 polynucleotides.

In some instances, each of the polynucleotides in the library are about 25 to 500 bases in length. In some instances, each of the polynucleotides in the library are about 25 to 50, 25 to 100, 25 to 150, 25 to 200, 25 to 250, 25 to 300, 25 to 350, 25 to 400, 25 to 450, 25 to 500, 50 to 100, 50 to 150, 50 to 200, 50 to 250, 50 to 300, 50 to 350, 50 to 400, 50 to 450, 50 to 500, 100 to 150, 100 to 200, 100 to 250, 100 to 300, 100 to 350, 100 to 400, 100 to 450, 100 to 500, 150 to 200, 150 to 250, 150 to 300, 150 to 350, 150 to 400, 150 to 450, 150 to 500, 200 to 250,200 to 300,200 to 350, 200 to 400,200 to 450,200 to 500, 250 to 300, 250 to 350, 250 to 400, 250 to 450, 250 to 500, 300 to 350, 300 to 400, 300 to 450, 300 to 500, 350 to 400, 350 to 450, 350 to 500, 400 to 450, 400 to 500, or 450 to 500 bases in length. In some instances, each of the polynucleotides in the library are about 25, 50, 100, 150, 200, 250, 300, 350, 400, 450, or 500 bases in length. In some instances, each of the polynucleotides in the library are at least about 25, 50, 100, 150, 200, 250, 300, 350, 400, or 450 bases in length. In some instances, each of the polynucleotides in the library are at most about 50, 100, 150, 200, 250, 300, 350, 400, 450, or 500 bases in length.

The libraries provided herein may store information. The information may be text information, audio information, visual information, or any combination thereof. In some instances, each of the plurality of polynucleotides in a library comprise a data block and a non-data block. The data block may comprise a portion of the item of information, while the non-data block may comprise identifiable information of the item in the data block. In some instances, the non-data block comprises metadata of the item of information. In some instances, the metadata comprises an index, data type, data size, data format, encryption codec, date of synthesis, date of last access, dates of previous handling, owner information, manufacture information, storage mechanism, or any combination thereof. In some instances, each of the plurality of polynucleotides or a portion of the plurality of polynucleotides comprises a barcode to order, sort, list, rank, or otherwise identify or organize the plurality of polynucleotides in the library. In some instances, alternatively or in addition to barcodes or non-data blocks in the plurality of polynucleotides, one or more containers holding the library may comprise a barcode or tag (e.g., RFID tag) comprising information to identify or organize the plurality of polynucleotides of the library.

The libraries provided herein may be proxy libraries or reference libraries to another one or more libraries encoding items of information. For example, the plurality of polynucleotides in a proxy library may comprise information about which other one or more libraries comprise items of information. The proxy library may be used to determine which other one or more libraries to retrieve to recover an item of information or a desired portion of the item of information. In some instances, the proxy library can be used to estimate error rate, uniformity, or both for a library encoding an item of information. The proxy library may further comprise metadata, such as those provided herein.

The libraries provided herein or a portion thereof may encode redundancy. In some instances, the portion that encodes for redundancy is about 10% to about 90%. In some instances, the portion that encodes for redundancy is about 20% to about 80%. In some instances, the portion that encodes for redundancy is about 20% to about 30%, about 20% to about 40%, about 20% to about 50%, about 20% to about 60%, about 20% to about 70%, about 20% to about 80%, about 30% to about 40%, about 30% to about 50%, about 30% to about 60%, about 30% to about 70%, about 30% to about 80%, about 40% to about 50%, about 40% to about 60%, about 40% to about 70%, about 40% to about 80%, about 50% to about 60%, about 50% to about 70%, about 50% to about 80%, about 60% to about 70%, about 60% to about 80%, or about 70% to about 80%. In some instances, the portion that encodes for redundancy is about 10%, about 20%, about 30%, about 40%, about 50%, about 60%, about 70%, about 80% or about 90%. In some instances, the portion that encodes for redundancy is at least about 10%, about 20%, about 30%, about 40%, about 50%, about 60%, about 70%, or about 80%. In some instances, the portion that encodes for redundancy is at most about 20%, about 30%, about 40%, about 50%, about 60%, about 70%, about 80%, or about 90%. In some instances, constructing a library comprising redox resistant bases decreases the redundancy that needs to be encoded in the plurality of polynucleotides to recover the item of information. In some instances, replacing one or more bases of a DNA sequence redox resistant bases decreases the redundancy that needs to be encoded in the plurality of polynucleotides to recover the item of information.

### Systems and Methods for Nucleic Acid Based Information Storage

Provided herein are devices, compositions, systems and methods for nucleic acid-based information (data) storage. The libraries provided herein comprising a redox resistant base (e.g., inosine) may be used to store information in DNA. In some instances, a library comprising a redox resistant base may increase the yield or fidelity, or decrease error rates of the polynucleotides of the library during synthesis or storage processes.

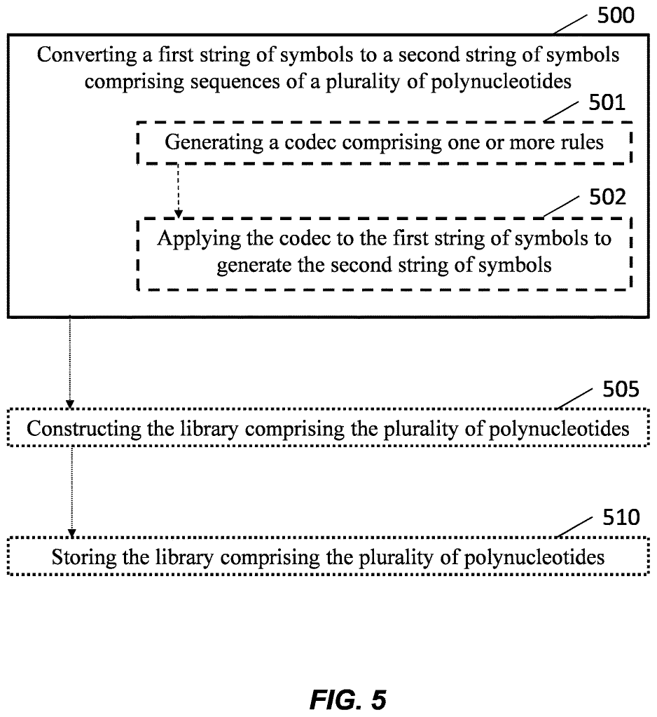

Provided herein are methods for storing an item of information in a plurality of polynucleotides (e.g., FIG. 5). As an example, optionally, a first string of symbols may be received. The first string of symbols may encode an item of information, such as a digital sequence encoding an item of information. In some instances, a first string of symbols comprises digital information in a binary code for processing by a computer. In some examples, methods for storing information comprise obtaining or receiving one or more items of information in the form of an initial code. In some instances, a first string of symbols is DNA sequence encoding an item of information. In some examples, the DNA sequence comprising canonical bases (e.g., A, T, C, G). In some instances, the DNA sequence does not comprise non-canonical bases, such as inosine.

Items of information include, without limitation, text, audio and visual information. Exemplary sources for items of information include, without limitation, books, periodicals, electronic databases, medical records, letters, forms, voice recordings, animal recordings, biological profiles, broadcasts, films, short videos, emails, bookkeeping phone logs, internet activity logs, drawings, paintings, prints, photographs, pixelated graphics, and software code. Exemplary biological profile sources for items of information include, without limitation, gene libraries, genomes, gene expression data, and protein activity data. Exemplary formats for items of information include, without limitation, .txt, .PDF, .doc, .docx, .ppt, .pptx, .xls, .xlsx, .rtf, .jpg, gif, .psd, .bmp, .tiff, .png, and .mpeg. The amount of individual file sizes encoding for an item of information, or a plurality of files encoding for items of information, in digital format include, without limitation, up to 1024 bytes (equal to 1 KB), 1024 KB (equal to 1 MB), 1024 MB (equal to 1 GB), 1024 GB (equal to 1 TB), 1024 TB (equal to 1 PB), 1 exabyte, 1 zettabyte, 1 yottabyte, 1 xenottabyte or more. In some instances, an amount of digital information is at least 1 gigabyte (GB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 gigabytes. In some instances, the amount of digital information is at least 1 terabyte (TB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 terabytes. In some instances, the amount of digital information is at least 1 petabyte (PB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 petabytes. In some instances, items of information are encoded using with an encoding scheme (e.g., 2 bits per base, 3 bits per base, or other encoding scheme).

The first string of symbols may be converted to a second string of symbols 500. The second string of symbols can comprise sequences of a plurality of polynucleotides, such as the plurality of polynucleotides in the libraries described herein. In some instances, the second string of symbols comprise sequences of polynucleotides comprising a redox resistant base. In some examples, the first string of symbols may be converted to a second string of symbols by generating a codec comprising one or more rules 501; and applying the codec to the first string of symbols to generate the second string of symbols 502. The one or more rules can comprise an error correction scheme, a codebook, a sequence constraint, or any combination thereof. In some instances, the error correction scheme can be used for spreading the digital or binary data to be stored over many polynucleotides. In some further embodiments, spreading the data also builds redundancy to correct errors. In some instances, spreading the data builds redundancy to correct for erasures (e.g., lost oligos). In some instances, the error correction scheme comprises a Reed-Solomon (RS) code, a linear error correction code (or linear block code), such as a low-density parity-check (LDPC) code, a linear block error-correcting code, such as polar code, a high-performance forward error correction (FEC), such as a Turbo-code, or any combination thereof (e.g., RS-based LDPC codes). In some instances, the codebook is used to map the first set of symbols to a second string of symbols based on one or more constraints. In some instances, the sequence constraint comprises one or more constraints related to length, inosine content, guanosine content, guanosine cytosine content, repeats of one or more bases, or any combination thereof. The second string of symbols comprising sequence of a plurality of polynucleotides can comprise at least one data block and at least one non-data block, as described herein.

In some instances, converting the first string of symbols to the second string of symbols comprises converting digital data of an item of information to sequences of polynucleotides comprising one or more redox resistant bases. In some instances, about 10% to 40% of bases in the sequences of polynucleotides comprise one or more redox resistant bases. In some instances, about 10% to 15%, 10% to 20%, 10% to 25%, 10% to 30%, 10% to 35%, 10% to 40%, 15% to 20%, 15% to 25%, 15% to 30%, 15% to 35%, 15% to 40%, 20% to 25%, 20% to 30%, 20% to 35%, 20% to 40%, 25% to 30%, 25% to 35%, 25% to 40%, 30% to 35%, 30% to 40%, or 35% to 40% of bases in the sequences of polynucleotides comprise one or more redox resistant bases. In some instances, about 10%, 15%, 20%, 25%, 30%, 35%, or 40% of bases in the sequences of polynucleotides comprise one or more redox resistant bases. In some instances, at least about 10%, 15%, 20%, 25%, 30%, 35%, or 40% of bases in the sequences of polynucleotides comprise one or more redox resistant bases. In some instances, at most about 10%, 15%, 20%, 25%, 30%, 35%, or 40% of bases in the sequences of polynucleotides comprise one or more redox resistant bases. In some instances, the sequences of the polynucleotides comprise adenosine, thymidine, cytidine, and inosine. In some instances, the sequences of the polynucleotides do not comprise guanosine. In some instances, all of the bases in the sequences of the polynucleotides have an oxidation potential of larger than that of dG.

In some instances, converting the first string of symbols to the second string of symbols comprises replacing one or more bases in a DNA sequences (e.g., a first string of symbols) encoding an item of information to with redox resistant bases to generate the sequences of polynucleotides comprising one or more redox resistant bases (e.g., second string of symbols). In some instances, about 10% to 40% of bases in the DNA sequence with the lowest oxidation potential are replaced. In some instances, about 10% to 15%, 10% to 20%, 10% to 25%, 10% to 30%, 10% to 35%, 10% to 40%, 15% to 20%, 15% to 25%, 15% to 30%, 15% to 35%, 15% to 40%, 20% to 25%, 20% to 30%, 20% to 35%, 20% to 40%, 25% to 30%, 25% to 35%, 25% to 40%, 30% to 35%, 30% to 40%, or 35% to 40% of bases in the DNA sequence with the lowest oxidation potential are replaced. In some instances, about 10%, 15%, 20%, 25%, 30%, 35%, or 40% of bases in the DNA sequence with the lowest oxidation potential are replaced. In some instances, at least about 10%, 15%, 20%, 25%, 30%, 35%, or 40% of bases in the DNA sequence with the lowest oxidation potential are replaced. In some instances, at most about 10%, 15%, 20%, 25%, 30%, 35%, or 40% of bases in the DNA sequence with the lowest oxidation potential are replaced. In some instances, all of the guanosine bases in the DNA sequence are replaced with inosine. In some instances, about 10% to 40% of the guanosine bases in the DNA sequence are replaced with inosine. In some instances, about 10% to 15%, 10% to 20%, 10% to 25%, 10% to 30%, 10% to 35%, 10% to 40%, 15% to 20%, 15% to 25%, 15% to 30%, 15% to 35%, 15% to 40%, 20% to 25%, 20% to 30%, 20% to 35%, 20% to 40%, 25% to 30%, 25% to 35%, 25% to 40%, 30% to 35%, 30% to 40%, or 35% to 40% of the guanosine bases in the DNA sequence are replaced with inosine. In some instances, about 10%, 15%, 20%, 25%, 30%, 35%, or 40% of the guanosine bases in the DNA sequence are replaced with inosine. In some instances, at least about 10%, 15%, 20%, 25%, 30%, 35%, or 40% of the guanosine bases in the DNA sequence are replaced with inosine. In some instances, at most about 10%, 15%, 20%, 25%, 30%, 35%, or 40% of the guanosine bases in the DNA sequence are replaced with inosine. In some instances, replacing one or more bases of a DNA sequence with a redox resistant base increases the fidelity of DNA encoding an item of information.

A plurality of polynucleotides comprising the second string of symbols may be constructed 505. The plurality of polynucleotides may be constructed on a solid support comprising a surface, such as those described herein, where the plurality of polynucleotides are extended from the surface comprising a plurality of structures.

In some instances, constructing a plurality of polynucleotides 505 comprises synthesizing the plurality of polynucleotides. As an example, a surface material for nucleic acid extension, a design for loci for nucleic acid extension (aka, arrangement spots), and reagents for nucleic acid synthesis can be selected. The surface of a structure can be prepared for nucleic acid synthesis, such as those described herein. The surface of a structure for polynucleotide synthesis may be configured for addressable control of regions of the surface. De novo polynucleotide synthesis can be performed. In some instances, synthesis comprises electrochemical deblocking using electrochemical acid generation. Electrochemical acid generation can comprise contacting a protected polynucleotide with a composition comprising one or more redox compounds. As an example, synthesis of polynucleotides can comprise one or more of: contacting a nucleoside attached to a solid support with a protected nucleoside; contacting the protected polynucleotide with a composition comprising one or more redox compounds; and applying a voltage to a solvent in fluid communication with the protected polynucleotide. In some examples, the protected nucleoside is configured to form a covalent bond with the nucleoside to generate a protected polynucleotide. In some examples, the voltage results in deprotection of the terminal nucleoside of the protected polynucleotide. The one or more redox compounds can comprise a substituted or unsubstituted quinone, such as those described herein. The composition can further comprise an organic salt and at least one solvent, as further described herein. The synthesized polynucleotides may be stored and available for subsequent release, in whole or in part.

In some instances, constructing a plurality of polynucleotides 505 comprises assembling the plurality of polynucleotides. Assembling the plurality of polynucleotides can comprise assembly by one or more building blocks and reagents. The one or more building blocks can comprise nucleic acid sequences. In some instances, the one or more building blocks are assembled in a fixed order. In some instances, the one or more building blocks are assembled in a random order. In some instances, the one or more building blocks are assembled using overlap-extension polymerase chain reaction (PCR), polymerase cycling assembly, sticky end ligation, biobricks assembly, golden gate assembly, gibson assembly, recombinase assembly, ligase cycling reaction, or template directed ligation. The assembled polynucleotides may be stored and available for subsequent release, in whole or in part.

Once the library comprising a plurality of polynucleotides are constructed, they may be stored 510. In some instances, the polynucleotides are stored in one or more compartments, each comprising the library or a portion thereof. In some examples, the one or more compartments are in fluidic and/or electronic communication. In some examples, the one or more compartments are not in fluidic and/or electronic communication. In some instances, the one or more compartments comprise a medium for storing the library of a portion thereof. The medium may be in a gas phase, liquid phase, or solid phase (e.g., salt solution at a molar ratio of less than 20:1 salt cation to phosphate groups in the DNA). In some instances, each of the one or more compartments comprises a barcode or tag (e.g., RFID tag) for identification (e.g., through metadata) the polynucleotides stored in each compartment. The one or more compartments may be stored in a storage system (e.g., racks or trays) described herein.

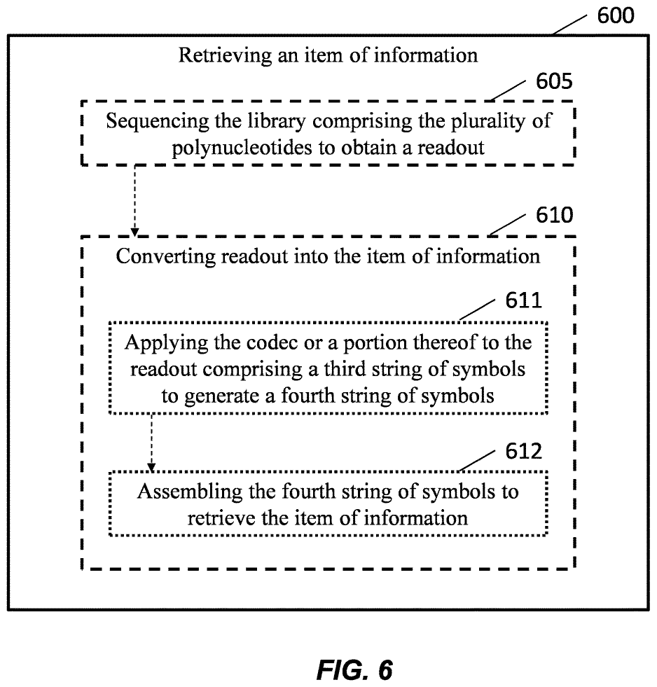

The information stored in polynucleotides using the system and methods described herein may be retrieved (e.g., FIG. 6). In some instances, once released, the polynucleotides, in whole or in part, are sequenced to obtain a readout 605. The readout may comprise a third string of symbols comprising the sequences of polynucleotides. In some instances, the readout is subject to decryption to convert sequences of polynucleotides back to the item of information 610. In some examples, converting the readout back into the item of information comprises applying the codec or a portion thereof to the readout comprising a third string of symbols to generate a fourth string of symbols 611. The fourth string of symbols can comprise a digital sequence. In some examples, the digital sequence can then assembled to obtain an alignment encoding for the original item of information 612. In some instances, retrieving the item of information comprises amplifying the polynucleotides. In some examples, the polynucleotides are amplified before sequencing. In some instances, the second string of symbols and the third string of symbols are at least 95%, 96%, 97%, 98%, 99%, 99.5%, 99.8%, 99.9%, 99.95%, or 99.99% identical. In some instances, the first string of symbols and the fourth string of symbols are at least 95%, 96%, 97%, 98%, 99%, 99.5%, 99.8%, 99.9%, 99.95%, or 99.99% identical. In some instances, the item of information is retrieved with at least 95%, 96%, 97%, 98%, 99%, 99.5%, 99.8%, 99.9%, 99.95%, or 99.99% accuracy.

### Nucleic Acid Synthesis

Provided herein are devices, methods, compositions, and systems for nucleic acid storage and synthesis. In some instance, nucleic acid storage or synthesis comprise a solid support. In some instances, solid supports comprise surfaces. Further provided herein are pluralities of devices which are combined to form larger arrays or chips. Further provided herein are devices and methods which are configured for electrochemical deprotection or deblocking during polynucleotide synthesis. In some instances, methods provided herein minimize voltages required for deblocking. Further provided herein are combinations of redox-active molecules for electrochemical acid generation. Further provided herein are combinations of redox-active molecules for voltage-controlled deblocking.

Conventional methods of electrochemical acid generation often require voltages in excess of those tolerable by high-density transistor devices (e.g., CMOS). Excess voltages in some instances give rise to unstable currents which reduce the fidelity of deblocking during polynucleotide synthesis. In some instances, fidelity of deblocking is increased by synthesizing polynucleotides comprising redox resistant bases, such as those provided herein. In some instances, methods described herein are configured to operate at voltages less than 2 volts. In some instances, methods described herein are configured for voltages of no more than 2.00, 1.95, 1.9, 1.85, 1.80, 1.75, 1.70, 1.65, 1.60, or no more than 1.50 volts. In some instances, methods described herein are configured for voltages of 0.1-2, 0.1-1.5, 1-1.9, 1-1.8, 1-1.7, 1-1.6 or 1-1.5 volts. In some instances, compositions described herein allow for reduced concentrations of redox compounds relative to previous methods. In some instances, compositions described herein allow for reduced concentrations of additives, such as reduced or eliminate concentrations of bases. In some instances, compositions described herein allow for reduced concentrations of additives, such as reduced or eliminate concentrations of amine bases (e.g., 2,6-lutidine). In some instances, an amine base, such as lutidine, is present at a concentration of about 0.1-10 mM. In some instances, an amine base is present at a concentration of 0.1-0.5 mM, 0.1-1 mM, 0.1-5 mM, 0.1-10 mM, 0.5-1 mM, 0.5-5 mM, 0.5-10 mM, 1-5 mM, 1-10 mM, or 5-10 mM. In some instances, an amine base is present at a concentration of 0.1, 0.5, 1, 5, or 10 mM. In some instances, an amine base is present at a concentration of at least 0.1, 0.5, 1, or 5 mM. In some instances, an amine base is present at a concentration of at most 0.5, 1, 5, or 10 mM.

Described herein are redox compositions which facilitate electrochemical acid generation. In some instances, a composition comprises one or more redox compounds. In some instances, a composition comprises a first redox compound and a second redox compound. In some instances, the first redox compound is a quinone or substituted derivative thereof. In some instances, the second redox compound is a hydroquinone or substituted derivative thereof. In some instances, a redox compound has the structure:

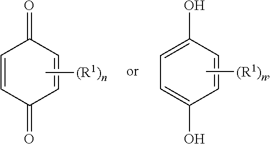

wherein
   each R¹ is independently halogen, —CN, —OR^{a}, —SR^{a}, —S(═O)R^{b}, —NO₂, —NR^{c}R^{d}, —S(═O)₂R^{d}, —NR^{a}S(═O)₂R^{d}, —S(═O)₂NR^{c}R^{d}, —C(═O)R^{b}, —OC(═O)R^{b}, —CO₂R^{a}, —OCO₂R^{a}, —C(═O)NR^{c}R^{d}, —OC(═O)NR^{c}R^{d}, —NR^{a}C(═O)NR^{c}R^{d}, —NR^{a}C(═O)R^{b}, —NR^{a}C(═O)OR^{a}, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen or —OR^{a}; and the cycloalkyl, heterocycloalkyl, aryl, and heteroaryl is optionally substituted with one, two, or three of halogen, C₁-C₆ alkyl, C₁-C₆ haloalkyl, —OR^{a}, or —NR^{c}R^{d}; or two or more R¹ are taken together to form a cycloalkyl, heterocycloalkyl, aryl, or heteroaryl ring;
R^{a} is hydrogen, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂;
R^{b} is C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂;
each R^{c} and R^{d} is independently hydrogen, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂;
or R^{c} and R^{d}, together with the nitrogen atom to which they are attached, form a heterocycloalkyl or heteroaryl; wherein the heterocycloalkyl and heteroaryl is optionally substituted with one, two, or three of halogen, C₁-C₆ alkyl, C—C₆ haloalkyl, —OH, —OMe, or —NH₂; and
n is 0-4.

In some instances, n is 0. In some instances, n is 1. In some instances, n is 2. In some instances, n is 3. In some instances, n is 4. In some instances, R¹ is independently selected from —CN, hydrogen, C₁-C₆ alkyl, halo, —OH, or —NH₂. In some instances, R¹ is independently Cl, F, Br, or I. In some instances, at least one R¹ is not hydrogen. In some instances, two or more R¹ are taken together to form a cycloalkyl, heterocycloalkyl, aryl, or heteroaryl ring. In some instances, two or more R¹ are taken together to form an aryl or heteroaryl ring. In some instances, a redox compound has the structure:

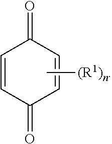

In some instances, a redox compound has the structure:

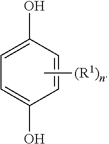

In some instances, a redox compound has the structure:

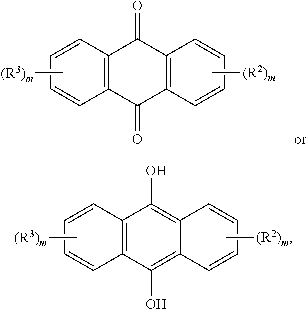

wherein
   each R² or R³ is independently hydrogen, halogen, —CN, —OR^{a}, —SR^{a}, —S(═O)R^{b}, —NO₂, —NR^{c}R^{d}, —S(═O)₂R^{d}, —NR^{a}S(═O)₂R^{d}, —S(═O)₂NR^{c}R^{d}, —C(═O)R^{b}, —OC(═O)R^{c}, —CO₂R^{a}, —OCO₂R^{a}, —C(═O)NR^{c}R^{d}. —OC(═O)NR^{c}R^{d}, —NR^{a}C(═O)NR^{c}R^{d}, —NR^{a}C(═O)R, —NR^{a}C(═O)OR^{a}, C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen or —OR^{a}; and the cycloalkyl, heterocycloalkyl, aryl, and heteroaryl is optionally substituted with one, two, or three of halogen, C₁-C₆ alkyl, C₁-C₆ haloalkyl, —OR^{a}, or —NR^{c}R^{d}; or two or more R² or R³ are taken together to form a cycloalkyl, heterocycloalkyl, aryl, or heteroaryl ring; R³ is hydrogen, C₁-C₆ alkyl, C₂-C₆ alkenyl, C₂-C₆ alkynyl, C₁-C₆ heteroalkyl. C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂;
   R^{b} is C₁-C₆ alkyl, C₂-C₆ alkenyl, C₂-C₆ alkynyl, C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂;
each R^{c} and R^{d} is independently hydrogen, C₁-C₆ alkyl, C₂-C₆ alkenyl, C₂-C₆ alkynyl. C₁-C₆ heteroalkyl, C₃-C₈ cycloalkyl, C₂-C₈ heterocycloalkyl, aryl, or heteroaryl; wherein the alkyl, alkenyl, alkynyl, and heteroalkyl is optionally substituted with one, two, or three of halogen, —OH, —OMe, or —NH₂; and the cycloalkyl, heterocycloalkyl, aryl, and heteroaryl is optionally substituted with one, two, or three of halogen, C₁-C₆ alkyl, C₁-C₆ haloalkyl, —OH, —OMe, or —NH₂;
or R^{c} and R^{d}, together with the nitrogen atom to which they are attached, form a heterocycloalkyl or heteroaryl; wherein the heterocycloalkyl and heteroaryl is optionally substituted with one, two, or three of halogen, C₁-C₆ alkyl, C₁-C₆ haloalkyl, —OH, —OMe, or —NH₂; and
m is 0-8.

In some instances, m is 0. In some instances, m is 1. In some instances, m is 2. In some instances, m is 3. In some instances, m is 4. In some instances, at least one R² is not hydrogen. In some instances, at least one R³ is not hydrogen. In some instances, R² or R³ is independently selected from —CN, hydrogen, C₁-C₆ alkyl, halo, —OH, or —NH₂. In some instances, R² or R³ is independently Cl, F, Br. or I. In some instances, a redox compound is selected from a compound of Table 1.

TABLE 1
Redox Compounds

| # | Oxidized Structure | # | Reduced Structure |
|---|---|---|---|
| 1A | 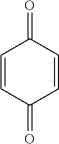 | 1B | 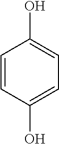 |
| 2A | 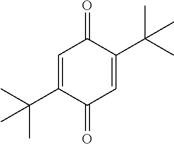 [SMILES: CC(C)(C)C1=CC(=O)C(C(C)(C)C)=CC1=O] | 2B | 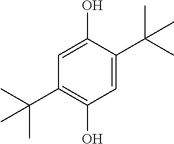 [SMILES: CC(C)(C)c1cc(O)c(C(C)(C)C)cc1O] |
| 3A | 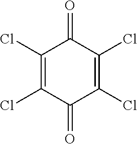 [SMILES: O=C1C(Cl)=C(Cl)C(=O)C(Cl)=C1Cl] | 3B | 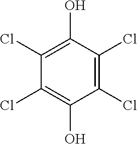 [SMILES: Oc1c(Cl)c(Cl)c(O)c(Cl)c1Cl] |
| 4A | 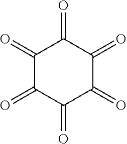 [SMILES: O=C1C(=O)C(=O)C(=O)C(=O)C1=O] | 4B | 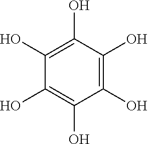 [SMILES: Oc1c(O)c(O)c(O)c(O)c1O] |
| 5A | 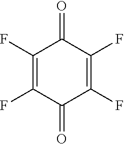 [SMILES: O=C1C(F)=C(F)C(=O)C(F)=C1F] | 5B | 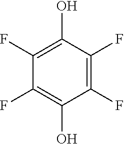 [SMILES: Oc1c(F)c(F)c(O)c(F)c1F] |
| 6A | 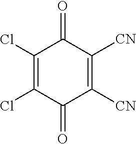 [SMILES: N#CC1=C(C#N)C(=O)C(Cl)=C(Cl)C1=O] | 6B | 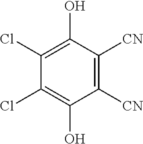 [SMILES: N#Cc1c(O)c(Cl)c(Cl)c(O)c1C#N] |
| 7A | 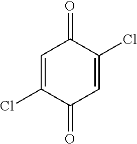 | 7B | 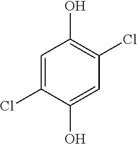 |
| 8A | 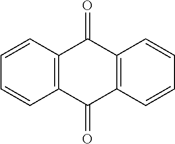 [SMILES: O=C1c2ccccc2C(=O)c2ccccc21] | 8B | 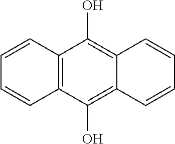 [SMILES: Oc1c2ccccc2c(O)c2ccccc12] |
| 9A | 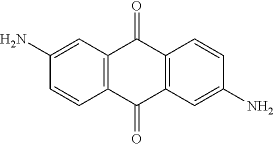 [SMILES: Nc1ccc2c(c1)C(=O)c1ccc(N)cc1C2=O] | 9B | 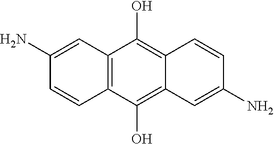 [SMILES: Nc1ccc2c(O)c3cc(N)ccc3c(O)c2c1] |

Redox compositions may comprise a first redox compound and a second redox compound e.g., redox “pair”). In some instances, a redox pair is selected from pairs E1-E43 in Table 3. In some instances, a first redox compound is selected the “B” column of Table 1. In some instances, a second redox compound is selected the “A” column of Table 1. In some instances, the first redox compound has the structure:

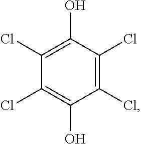 [SMILES: Oc1c(Cl)c(Cl)c(O)c(Cl)c1Cl]

and the second redox compound has the structure

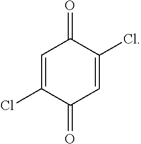

In some instances, the first redox compound has the structure:

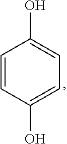

and the second redox compound has the structure

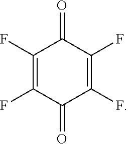 [SMILES: O=C1C(F)=C(F)C(=O)C(F)=C1F]

In some instances, the first redox compound has the structure:

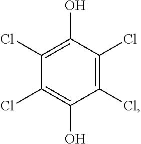 [SMILES: Oc1c(Cl)c(Cl)c(O)c(Cl)c1Cl]

and the second redox compound has the structure

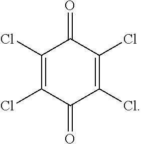 [SMILES: O=C1C(Cl)=C(Cl)C(=O)C(Cl)=C1Cl]

In some instances, first redox compound has the structure:

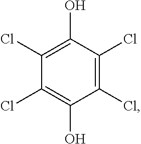 [SMILES: Oc1c(Cl)c(Cl)c(O)c(Cl)c1Cl]

and the second redox compound has the structure

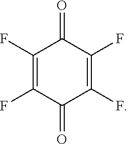 [SMILES: O=C1C(F)=C(F)C(=O)C(F)=C1F]

In some instances, the first redox compound and the second redox compound are selected such that a redox potential is less than 2 volts. In some instance, a first redox compound is present in a reduced state. In some instance, a second redox compound is present in an oxidized state. In some instances, redox compositions described herein further comprise a plurality of polynucleotides. In some instances, at least some of the polynucleotides comprise acid-sensitive deblocking groups.

The concentration of a redox compound may be varied to achieve a desired redox potential. In some instances, a redox compound is present at 0.1-1M. In some instances, a redox compound is present at 0.0002-1, 0.0002-0.01, 0.001-0.01, 0.001-0.01, 0.001-1, 0.01-0.1, 0.01-0.5, 0.01-1, 0.1-0.5, 0.1-0.9, 0.3-0.7, 0.4-0.6, 0.4-1, 0.5-1, or 0.6-1M. In some instances, a redox compound is present at about 0.0002, 0.001, 0.005, 0.01, 0.05, 0.1, 0.2, 0.3, 0.4, 0.5, 0.6, 0.7, 0.8, 0.9, or about 1M. In some instances, a redox compound is present at no more than 0.005, 0.01, 0.1, 0.2, 0.3, 0.4, 0.5, 0.6, 0.7, 0.8, 0.9, or no more than 1M. In some instances, a first redox compound is present at 0.0002-0.01, 0.001-0.01, 0.001-0.01, 0.001-1, 0.01-0.1, 0.1-0.5, 0.1-0.9, 0.3-0.7, 0.4-0.6, 0.4-1, 0.5-1, or 0.6-1M. In some instances, a first redox compound is present at 0.001, 0.005, 0.01, 0.05, 0.1, 0.2, 0.3, 0.4, 0.5, 0.6, 0.7, 0.8, 0.9, or about 1M. In some instances, a second redox compound is present at 0.0002-1, 0.0002-0.01, 0.001-0.01, 0.001-0.01, 0.001-1, 0.01-0.1, 0.1-0.5, 0.1-0.9, 0.3-0.7, 0.4-0.6, 0.4-1, 0.5-1, or 0.6-1M. In some instances, a second redox compound is present at 0.0002, 0.001, 0.005, 0.01, 0.05, 0.1, 0.2, 0.3, 0.4, 0.5, 0.6, 0.7, 0.8, 0.9, or about 1M. In some instances, the first redox compound and the second redox compound are present at a ratio of about 1:1, 5:1, 10:1, 50:1, or 100:1. In some instances, the first redox compound and the second redox compound are present at a ratio of about 1:1 to 5:1, 1:1 to 10:1, 1:1 to 50:1, 1:1 to 100:1, 5:1 to 10:1, 5:1 to 50:1, 5:1 to 100:1, 10:1 to 50:1, 10:1 to 100:1, or 50:1 to 100:1. In some instances, the first redox compound and the second redox compound are present at a ratio of at least about 1:1, 5:1, 10:1, or 50:1. In some instances, the first redox compound and the second redox compound are present at a ratio of at most about 5:1, 10:1, 50:1, or 100:1. In some examples, the first redox compound is hydroquinone and the second redox compound is benzoquinone.

The redox compositions described herein may comprise additional components. In some instances, a redox composition comprises a salt. In some instances, the salt is an organic salt. In some instances, the organic salt is a phase-transfer catalyst. In some instances, the organic salt comprises a tetra alkyl ammonium cation. In some instances, the organic salt comprises an tetrabutylammonium cation. In some instances, the organic salt comprises an tetramethylammonium cation. In some instances, the organic salt comprises an hexafluorophosphate cation. In some instances, the organic salt comprises hexafluorophosphate, tetrafluoroborate, tetraphenylborate, hexafluorophosphate, perchlorate, tetrachloroferrate, hexafluoroarsenate, hexafluoroantimonate, pentafluorohy-droxyantimonate, hexachloroantimonate, tetrakispentafluorophenylborate, toulenesulfonate, tetrakis-(pentafluoromethylphenyl) borate,bi(trifluoromethylsulphonyl)amides or tris(trifluoromethylsulphonyl)methides. In some instances, the salt is tetraethylammoniump-toulenesulfonate (TEA-PTS). In some instances, the salt is tetrabutylammonium hexaflorophosphate (TBA-HFP). In some instances, the concentration of a salt (e.g., organic salt) is 10-100 mM. In some instances, the concentration of a salt (e.g., organic salt) is 10-50 mM. In some instances, the concentration of a salt (e.g., organic salt) is 10-50 mM, 5-100 mM, 15-75 mM, 25-100 mM, 25-50 mM, 15-25 mM, or 15-50 mM. In some instances, the concentration of a salt (e.g., organic salt) is about 5, 10, 15, 17, 20, 25, 30, 35, 40, 45, 50, 55, 60, 65, 70, 75, 80, 85, 90, 95 or about 100 mM. In some instances, the concentration of a salt (e.g., organic salt) is about 17 mM. In some instances, the concentration of a salt (e.g., organic salt) is about 25 mM. In some instances, the concentration of a salt (e.g., organic salt) is about 50 mM. In some instances, the concentration of a salt (e.g., organic salt) is about 100 mM. In some instances, the concentration of a salt (e.g., organic salt) is no more than 5, 10, 15, 17, 20, 25, 30, 35, 40, 45, 50, 55, 60, 65, 70, 75, 80, 85, 90, 95 or no more than 100 mM.

The redox compositions described herein may comprise at least one solvent. In some instances, the solvent is an organic solvent. In some instances, the solvent comprises hydrocarbons (e.g., hexane, decane, benzene, toluene, xylene, isomers thereof, and the like), ethers (e.g., THF, diethyl ether, methyl t-butyl ether, and the like), esters (e.g., methyl acetate, ethyl acetate, tert-butylacetate, etc.), lactones, ketones (e.g., acetone, methyl ethyl ketone, cyclopentanone, and the like), alcohols (e.g., ethanol, butanol, isopropanol, and the like), amides (e.g., DMF, N-methylpyrrolidinone, or other amides), ureas, carbonates (e.g., diethylcarbonate, or other carbonate), carbamates, aldehydes, amines, cyanates, isocyanates, sulfoxides, sulfones, aromatics, heteroaromatics, thiols, phosphoramides, nitriles (e.g., acetonitrile), alkynes, alkenes, alkanes, halogenated solvents (e.g., tetrachloromethane, dichloromethane, chloroform, or other halogenated solvent), silanes, perfluorocarbons (C₂-C₁₈ perfluorinated branched or straight alkanes such as perfluorohexane, perfluoroheptane, perfluorodecane, perfluoro aromatics such as perfluorobenzene, or other perfluorocarbon), supercritical fluids, ionic liquids, compressed gases, and the like. In some instances, solvents comprise a nitrile, such as acetonitrile. In some instances, solvents comprise an alcohol, such as methanol or ethanol. In some instances, solvents comprise a halogenated solvent, such as dichloromethane, chloroform, or 1,2-dichloromethane. Any of the solvents described herein may be present as a mixture. In some examples, the solvent comprises tetrahydrofuran. In some examples, the solvent comprises acetonitrile. In some examples, the solvent comprises acetonitrile and one of more of methanol, ethanol, i-propanol, dimethylsulfoxide, dioxane, ethyleneglycol, N, N-dimethylformamide, or tetrahydrofuran. In some instances, a mixture of solvents comprises at least two solvents. In some examples, a mixture comprising acetonitrile and a second solvent comprises more acetonitrile than the second solvent. In some examples, two solvents are present in a ratio of about 1:1, 2:1, 3:1, 4:1, 5:1, 6:1, 7:1, 8:1, 9:1, 10:1, 11:1, 12:1, 13:1, 14:1, 15:1, 19:1, 20:1, 25:1, 49:1, 99:1, 3:2, 5:2, 7:2, 9:2, 11:2, or 13:2.

Compositions described herein may be substantially free from bases. In some instances, the base is a non-nucleophilic base. In some instances, base is an amine base. Non-limited examples of amine bases include 2,6-lutidine, diisopropylethylamine, 1,8-diazabicyclo[5.4.0]undec-7-ene (DBU), 1,5-Diazabicyclo(4.3.0)non-5-ene (DBN), 2,6-Di-tert-butylpyridine, pyridine, or other amine base. In some instances the base is a phosphazene base, such as t-Bu-P₄.

Redox compositions described herein may result in higher levels of spatial control over deblocking. In some instances, neighboring (e.g., adjacent) loci for polynucleotide synthesis which are not subjected to deblocking voltages are not deblocked. In some instances, adjacent loci are deblocked less than 1%, 0.5%, 0.2%, 0.1%, 0.05%, 0.01%, 0.005%, 0.001% or less than 0.0001%.

### Synthesis Devices

Provided herein are devices for polynucleotide synthesis. Such devices in some instances comprise a solid support comprising a plurality of features (or loci) for polynucleotide synthesis. Such devices may comprise conductive elements or electrodes. Such electrodes may function as anodes or cathodes. Polynucleotides comprise a protecting or blocking group bound to a terminal base during at least one synthesis cycle. Application of a voltage through electrodes during a synthesis step in some instances generates a deprotection reagent (such as an acid or other deprotection reagent) which deblocks polynucleotides (removes the protection group). The length of time the voltage is applied, number of times it applied, and other variables have a significant effect on the extent of deprotection, speed of deprotection, and reduction in unwanted side reactions caused by excess deblocking reagents. The geometry of the device's surface and electrodes also may influence the efficiency of the deblocking step.

Provided herein are devices for polynucleotide synthesis comprising layers of materials. Such devices may comprise any number of layers of materials comprising conductors, semiconductors, or insulative materials. Traditional devices comprise a base layer, conducting materials (one or more conducting layers configured for use as an electrode; conducting materials may be buried in the base layer or above the base layer), and a porous growth layer surface. In some instances, conductive layer is in electrical contact with layer. Each of such layers may be individually patterned to generate features for polynucleotide synthesis such as pores, holes, wells, channels, or other shape. Various layers of such devices are in some instances combined to form addressable solid supports. Layers or surfaces of such devices may be in fluid communication with solvents, solutes, or other reagents used during polynucleotide synthesis. Further described herein are devices comprising a plurality of surfaces. In some instances, surfaces comprise features for polynucleotides synthesis in proximity to conducting materials. In some instances, devices described herein comprise 1, 2, 5, 10, 50, 100, or even thousands of surfaces per device. In some instances, a voltage is applied to one or more layers of a device described herein to facilitate polynucleotide synthesis. In some instances, a voltage is applied to one or more layers of a device described herein to facilitate a step in polynucleotide synthesis, such as deblocking. Different layers on different surfaces of different devices are often energized with a voltage at varying times or with varying voltages. For example, a positive voltage is applied to a first layer, and a negative voltage is applied to a second layer of the same or a different device. In some instances, one or more layers on different devices are energized, while others are disconnected from a ground. In some instances, base layers comprise additional circuitry, such as complementary metal-oxide-semiconductors (CMOS) devices. In some instances, various layers of one or more devices are connected laterally via routing, and/or vertically with vias. In some instances, various layers of one or more devices are connected laterally via routing, and/or vertically with vias to a CMOS layer. In some instances, various layers of one or more devices are connected to a CMOS device via wire bonds, pogo pin contacts, or through Si Vias (TSV).

A first device provided herein comprises a base layer, and a patterned top layer. In some instances, the top layer comprises a conducting material (conducting layer). In some instances, a polynucleotide synthesis surface is formed on the solvent-exposed surface of the base layer. Such a device provides fluid communication between the polynucleotide synthesis surface and the top layer.

A second device provided herein comprises a base layer, a buried shield electrode, and a patterned top layer. In some instances, the top layers and comprise a conducting material. In some instances, devices comprise a conducting layer present in the base layer. In some instances, a polynucleotide synthesis surface is formed by pores in the top layer. Such a device provides fluid communication between the polynucleotide synthesis surface and the top layer. In some instances, the buried shield electrode does not contact the synthesis surface or top layer. In some instances, voltage is passed through the shield electrode to influence the flow of ions in a solvent which contacts the synthesis surface. In some instances a different voltage is applied to the shield electrode compared to the voltage applied to the top layer. In some instances, a voltage applied to the shield electrode is synchronized with an adjacent or proximate conducting layer. In some instances, the time between a voltage applied to the shield electrode and the proximate anode is no more than 0.001 microseconds, 0.1, 0.2, 0.5, 0.8, 1.0, 1.2, 1.5, 1.8, 2, 5, 8, 10, 12, 15, 20, 50, 80, or no more than 100 microseconds. In some instances, the time between a voltage applied to the shield electrode and the proximate anode is no more than 0.001 seconds, 0.1, 0.2, 0.5, 0.8, 1.0, 1.2, 1.5, 1.8, 2, 5, 8, 10, 12, 15, 20, 50, 80, or no more than 100 seconds. In some instances, the time between a voltage applied to the shield electrode and the proximate anode is no more than 0.1 microsecond, 0.2, 0.5, 0.8, 1.0, 1.2, 1.5, 1.8, 2, 5, 8, 10, 12, 15, 20, 50, 80, or no more than 100 microseconds. In some instances, the time between a voltage applied to the shield electrode and the proximate anode is about 0.1 microsecond, 0.2, 0.5, 0.8, 1.0, 1.2, 1.5, 1.8, 2, 5, 8, 10, 12, 15, 20, 50, 80, or about 100 microseconds. In some instances, the time between a voltage applied to the shield electrode and the proximate anode is 0.1-1, 0.1-5, 0.1-10, 0.1-100, 0.5-10, 0.5-100, 1-10, 1-50, 1-100, 5-50, 10-100 or 50-100 microseconds.

A third device provided herein comprises a base layer, and an intermediate layer, and a top layer. In some instances, the intermediate layer and layer comprise a conducting material. In some instances, the top layer comprises a polynucleotide synthesis surface. Such a device provides fluid communication between the polynucleotide synthesis surface and the intermediate layer. The polynucleotide synthesis surfaces in some instances are patterned as cylinders, substantially rectangular shapes, channels, or other shape. In some instances, polynucleotide synthesis surfaces are randomly distributed. In some instances, the intermediate layer comprises a thermal oxide. Devices in some instances comprise one or more additional bonding layers between the synthesis surface and the bottom layer.

A fourth device provided herein comprises a base layer, a first intermediate layer, a top layer. In some instances, the first intermediate comprises a polynucleotide synthesis surface. In some instances, the smallest feature dimension is. In some instances, the smallest feature dimension is proportional to the diffusion distance of a reagent generated proximate to a conducting layer.

A fifth device provided herein comprises a base layer, a first intermediate layer, a second intermediate layer, a top layer. In some instances, polynucleotides are synthesized on top layer. The polynucleotide synthesis surfaces in some instances are patterned as cylinders, substantially rectangular shapes, channels, or other shape. In some instances, polynucleotide synthesis surfaces are randomly patterned. In some instances, the smallest feature dimension is. In some instances, a device comprises additional bonding layers. In some instances, the smallest feature dimension is proportional to the diffusion distance of a reagent generated proximate to a conducting layer. In some instances, a device comprises a conductive layer configured for use as a cathode which is above the plane of one or more conductive layers configured for use as an anode (attached to a lower conductive layer). In some instances, the anode is in fluid communication with one or more loci for polynucleotide synthesis. In another configuration, a conductive layer configured for use as a cathode and located in the same plane as one or more conductive layers configured for use as an anode (attached to a lower conductive layer).

A sixth device provided herein comprises a plurality of addressable solid supports, which are in fluid communication with the flow cell area. In some instances, such a device is used to evaluate operational variables of the device. Each surface comprises a plurality of features for polynucleotide synthesis surrounded by a conducting layer. In some instances, a device comprises at least 1, 2, 5, 10, 20, 50, or more than 50 addressable solid supports. In some instances, the surfaces comprise a series of patterned features such as pores or wells for polynucleotide synthesis. The smallest feature dimension in some instances is the diameter of the wells and/or the distance between wells. In some instances, a device comprises at least 1, 2, 5, 10, 20, 50, or more than 50 addressable solid supports. In some instances, the surfaces comprise a series of patterned features such as channels for polynucleotide synthesis. The smallest feature dimension in some instances is the width of the channels and/or the distance between channels. The smallest feature dimension in some instances is the width of the channels and/or the distance between channels. In some instances, surfaces are located on a device such to maximize the available surface area. In some instances, the distance between any two surfaces is 5-1000 microns, 10-500, 50-500, 5-100, 3-10, 3-50, 25-500 or 50-1000 microns. Additional patterns of features are also in some instances used with the devices described herein. In some instances, the pattern of features on a device are random.

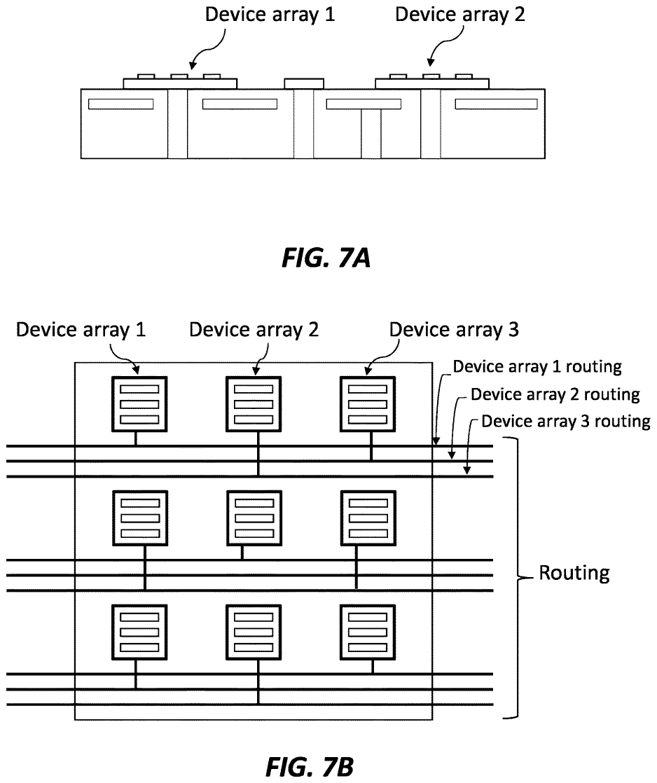

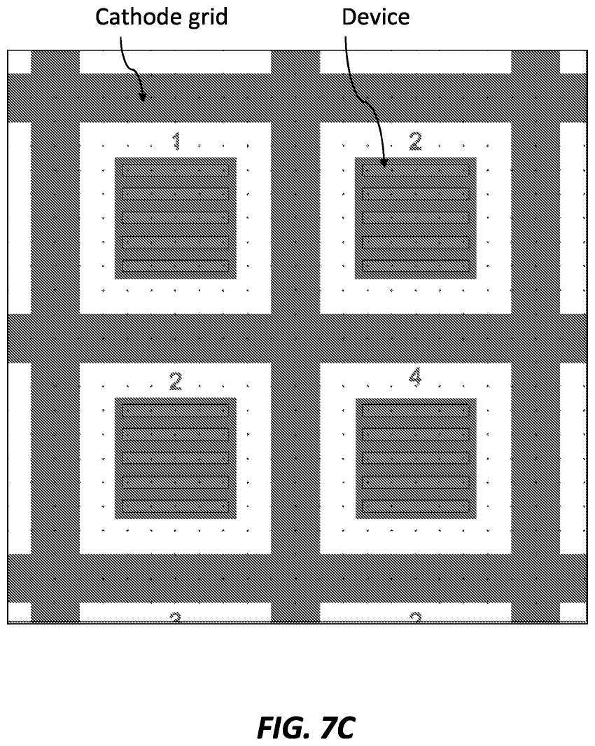

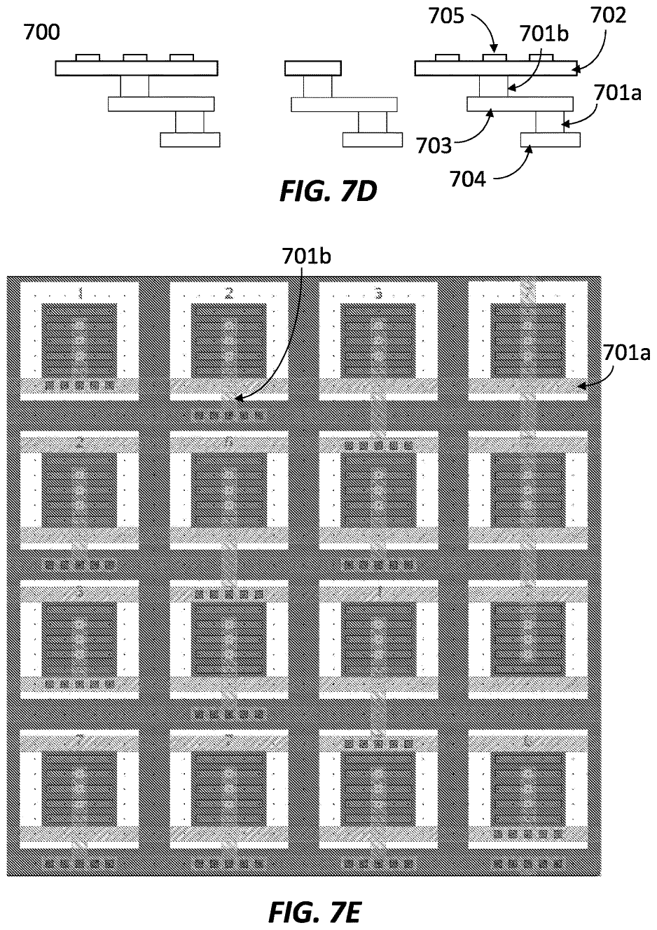

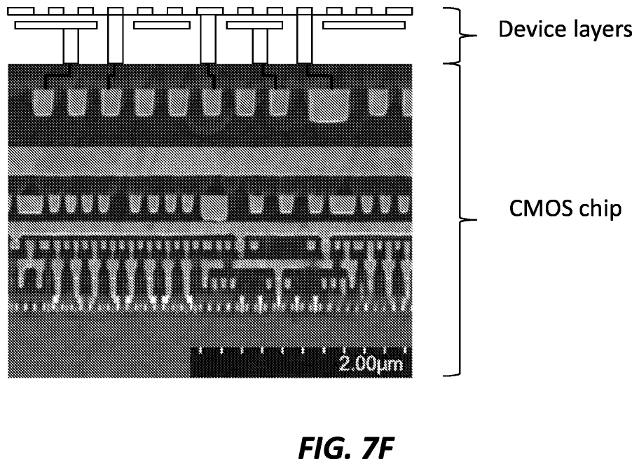

A seventh device described herein comprises a plurality of device arrays (or addressable solid supports), as shown in FIGS. 7A-7E. FIG. 7A shows two such device arrays for clarity, although such devices may comprise any number of device arrays. Nine such device arrays are shown in FIG. 7B, along with routing connections which allow addressable control of individual or groups of device arrays. The system in FIG. 7B comprises three rows (top, middle, bottom) and three columns (left, center, right). Device array 1 is shown at the top left, middle center, and bottom right. Device array 2 is shown at the top center, middle left, middle right, and bottom center. Device array 3 is shown at the top right and bottom left. The routing connections for each row are shown in the order of device array 1 routing, device array 2 routing, and device array 3, routing. In some instances, device array 1 is individually addressable from device arrays 2 and 3. Four such device arrays are shown in FIG. 7C for clarity only; any number of devices may be arrayed in this way. The four devices are addressable in groups 1, 2, and 4 as shown for clarity only: the number of addressable groups in some instances is equal to or less than the number of total device arrays. Conductive layers 702 in some instances generate reagents (e.g., acid) for electrochemical deprotection of biomolecules, such as polynucleotides. In some instances, 702 is configured for use as an anode. In some instances, a conducting layer is also configured for use as a cathode as shown in FIG. 7C. A cross section of devices of FIG. 7C or 7E are shown in FIG. 7D. In some instances, polynucleotides are synthesized on oxide layer 705. In some instances, polynucleotides are synthesized on conductive layer 702. Such devices are in some instances addressable by a first layer of routing 701a and a second layer of routing 701b. Such devices may comprise any number of routing layers, for example 1, 2, 3, 4, 5, 10, 20, 50, 100, or more than 100 layers of routing. Routing in different horizontal planes in some instances is connected by one or more vertical interconnect accesses (VIAs), 703 and 704. Such devices may comprise any number of vias, for example 1, 2, 3, 4, 5, 10, 20, 50, 100, 1000, or more than 1000 vias per square micron. The number and size of routing and vias in some instances are proportional to the number of addressable solid supports on a device. A device comprising 16 device arrays (similar to FIG. 7C) is shown in FIG. 7E. Routing 701b is superficial to routing 701a in the device. The sixteen device arrays of FIG. 7E are in some instances addressable as seven groups, but other configurations are also consistent with the devices and methods described herein.

Devices may comprise any number of device arrays. In some instances, devices comprise at least 10, 50, 100, 1000, 10,000, 100,000, or more than 100,000 device arrays in a single device. In some instances, devices comprise about 10, 50, 100, 1000, 10,000, 100,000, or about 100,000 device arrays in a single device. In some instances, devices comprise 10-50, 10-5000, 10-10,000, 100-1000, 100-10,000, 100-100,000, 1000-10,000, or 1000-100,000 device arrays in a single device.

Device arrays may be scaled to any size or dimensions. In some instances, device arrays are about 0.01, 0.02, 0.05, 0.1, 0.2, 0.5, 0.8, 1, 2, 5, 8, or about 10 microns in width. In some instances, device arrays are no more than 0.01, 0.02, 0.05, 0.1, 0.2, 0.5, 0.8, 1, 2, 5, 8, or no more than 10 microns in width. In some instances, device arrays are 0.01-10, 0.1-10, 0.1-1, 0.5-1, 1-10, or 5-30 microns in width. In some instances, device arrays are separated by about 0.01, 0.02, 0.05, 0.1, 0.2, 0.5, 0.8, 1, 2, 5, 8, or about 10 microns. In some instances, device arrays are separated by no more than 0.01, 0.02, 0.05, 0.1, 0.2, 0.5, 0.8, 1, 2, 5, 8, or no more than 10 microns. In some instances, device arrays are separated by 0.01-10, 0.1-10, 0.1-1, 0.5-1, 1-10, or 5-30 microns.

Devices with addressable device arrays may be addressed in different patterns or configurations. In some instances, only specific groups of device arrays are activated simultaneously. In some instances, device arrays are addressed according to. Any number of device arrays may be activated simultaneously. In some instances, about 1%, 2%, 5%, 10%, 20%, 50%, 75%, or about 100% of the device arrays in a device described herein are activated simultaneously. In some instances, no more than 1%, 2%, 5%, 10%, 20%, 50%, 75%, or no more than 99% of the device arrays in a device described herein are activated simultaneously. In some instances, at least 1%, 2%, 5%, 10%, 20%, 50%, 75%, or at least 99% of the device arrays in a device described herein are activated simultaneously.

Devices described herein may be fabricated using numerous methods, such as masking methods. In some instances, a lift-off fabrication method is used. Lift-off methods in some instances comprises addition of a sacrificial layer (e.g., photoresist or “PR”) to a base layer coated with an oxide layer, addition of a conductive layer, and removal of the sacrificial layer. In some instances, a dry-etch fabrication method is used. Dry-etch methods in some instances comprises addition of one or more layers to a base layer, such as an oxide layer, a first intermediate layer (e.g., TiN, or other material), a conductive layer (e.g., platinum), a second intermediate layer (e.g., TiN, or other material), and a sacrificial layer (e.g., photoresist); partial removal of the second intermediate layer to expose the conductive layer; partial removal of the conductive layer to expose the first intermediate layer; partial removal of the first conductive layer to expose the first intermediate layer; and partial removal of the first intermediate layer to expose the oxide layer.

### Electrochemistry

Provided herein are methods of applying voltage to devices with the redox compositions described herein. Such voltages may result in any number of different effects, such as electrochemical reaction with solvents or solutes. In some instances, apply a voltage results in deblocking or removal of protecting groups on molecules attached to a synthesis surface. In some instances molecules are polysaccharides, polynucleotides, polypeptides, or other polymer. In some instances, apply a voltage results in deblocking nucleic acids using the devices described herein. Further provided herein are methods wherein the devices described herein are energized with an electrical voltage. In some instances, the electrical voltage is used to deblock oligonucleotides bound to a solid support or surface. Such deblocking in some instances occurs through direct electrochemical reaction of a blocking group on a polynucleotide, or through the generation of deblocking reagents, such as an acid.

Methods described herein may comprising energizing a device with a voltage (applying a voltage) for a period of time. Applied voltages in some instances form a circuit between a cathode and an anode, leading to current flow through the device, solvent, and/or other components. In some instances, a layer of a device is configured as an anode or cathode. In some instances, a device comprises an anode which is located above the plane of a cathode (“sandwiched”). In some instances, conductive layer is in electrical contact with layer. In some instances, a device comprises an anode which is located in substantially the same plane of the cathode. Application of voltage in some instances is configured to perform a step of polynucleotide synthesis. In some instances, methods comprise application of a voltage to deblock polynucleotides. In some instances application of a voltage generates a deblocking reagent. In some instances, devices comprise conducting layers in fluid communication with a solvent, wherein the solvent comprises reagents which generate a deblocking reagent. In some instances, the deblocking reagent is an acid. In some instances, the acid is H⁺.

Methods described herein may comprise applying a voltage to one or more devices described herein. In some instances, such voltages result in deprotection of molecules (polynucleotides, polypeptides, polysaccharides, or other polymer) at one or more devices or regions. In some instances, application of a voltage at one or more devices results in deprotection of polynucleotides at one or more devices or regions within one or more devices. In some instances, a device is described as “inactive” if a deprotection reagent (e.g., acid or other reagent) is not generated at or in the vicinity of a device or region of a device. In some instances, a device is described as “active” if a deprotection reagent (e.g., acid or other reagent) is generated at or in the vicinity of a device or region of a device. In some instances, deprotection of polynucleotides occurs at or near one or more active devices, or regions of one or more active devices. In some instances, both active and inactive devices are energized with voltages. In some instances, voltage is applied to inactive devices in levels which are insufficient to generate a deprotection reagent. In some instances, deprotection comprises application of one or more voltages (or voltage levels) for periods of time. In some instances, a single voltage level is used for deprotection of polynucleotides. In some instances, more than one voltage level is used during deprotection. In some instances, a cathode voltage is kept constant at 0V, while the anode voltage is increased from 0V to 2V during a “pulse”. In some instances, a cathode voltage is kept constant at a negative voltage (e.g., −1V or other negative voltage), while the anode voltage is increased from 0V to 2V during a “pulse”. In some instances, a cathode voltage is decreased from 0V to a negative voltage (such as −1V), and an anode voltage is increased from 0V to 1V during a “pulse”. In some instances such voltages are synchronized, wherein the decrease in voltage at the cathode and increase in voltage at the anode occur at approximately the same time. In some instances such voltages are synchronized, wherein the decrease in voltage at the cathode and increase in voltage at the anode occur within 1 sec, 0.5 sec, 0.1 sec, 0.05 sec, 0.01 sec, 0.005 sec, 0.001 sec, or occur within 0.0005 sec of each other. In some instances, two voltage levels are used during a deprotection step. In some instances, two voltage levels are used during a deprotection step, e.g., a positive and neutral voltage. In some instances, two voltage levels are used during a deprotection step, e.g., a positive and negative voltage. In some instances, three voltage levels are used during a deprotection step, e.g., a positive, neutral (or zero/about zero), and negative voltage. Voltage in some instances is applied to multiple electrodes in fluid communication with the same surface, for example between a deblocking electrode and a shield electrode. Voltages between the deblocking electrode and shield electrode are in some instances are synchronized. In some instances, when the difference between the cathode and anode voltages exceeds a threshold, acid or other reagent is generated. In some instances, synchronizing positive anode and negative cathode voltages results in the advantage of reducing the magnitude of the voltages that are necessary to drive a device.

Devices may be described as circuits between an anode and a cathode. In some instances, such circuits are described as being in device states, such as “on”, “off”, or “alternate resistance”. In some instances, alternate resistance is a high resistance state, or “disconnect” state. In some instances, a high resistance state is a resistance state that is higher than an off state (e.g., low/no voltage in off state, but still connected to a ground). In some instances, a high resistance state provides an effective amount of resistance to reduce current flow through one or more inactive devices. Without being bound by theory, the disconnect state in some instances reduces undesired deprotection at areas adjacent to an on device. In some instances, a high resistance state provides an effective amount of resistance to reduce current flow to near zero in one or more inactive devices. In some instances an off state is generated by zero (or near zero) voltage between an inactive device and a common cathode. In some instances an off state exists even with a minimum voltage applied between an inactive device and a common cathode, wherein the minimum voltage is below that amount needed for deprotection. In some instances a high resistance state is generated by zero voltage between an inactive device and the cathode and a higher resistance between the inactive devices and a common cathode. In some instances, an off state indicates zero voltage or negative voltage between the anode and active device (cathode). In some instances, an on state indicates positive voltage between the anode and active device (cathode) which is sufficient for deprotection. In some instances, an inactive device is in the off or alternate resistance state. In some instances, an active device (where deprotection is desired) is cycled (pulsed) between one or more on and off states for a period of time. In some instances, an active device (where deprotection is desired) is cycled between one or more on and off states for a period of time and neighboring inactive devices are maintained in an alternative resistance state.

A voltage may be applied to the cathode in addition to the anode. In some instances, the cathode is biased with a negative voltage relative to ground. In some instances, biasing the voltage (bias voltage) of the cathode reduces the maximum anode voltage needed for electrochemical deprotection (e.g., the voltage difference between the anode and cathode will equal the anode voltage plus the magnitude of the negative bias voltage at the cathode). In some instances, a device comprises a contact bias on the cathode. In some instances, a bias voltage at the cathode is switched whenever the anode voltage is switched (e.g., synched). In some instances, a cathode controls electrochemistry for a single device. In some instances, a cathode controls electrochemistry for a plurality of devices (“common” cathode). In some instances, use of a common cathode results fewer transistors needed per device. In some instances, the bias voltage is no more than −0.1, −0.2, −0.3, −0.5, −0.7, −0.9, −1.0−1.1, −1.2, −1.5, −1.8, −2.0, −2.1, −2.2, or no more than −2.5 volts. In some instances, the biased voltage is at least −0.1, −0.2, −0.3, −0.5, −0.7, −0.9, −1.0−1.1, −1.2, −1.5, −1.8, −2.0, −2.1, −2.2, or at least −2.5 volts. In some instances, the biased voltage is about −0.1, −0.2, −0.3, −0.5, −0.7, −0.9, −1.0−1.1, −1.2, −1.5, −1.8, −2.0, −2.1, −2.2, or about −2.5 volts. In some instances, the biased voltage is −0.1 to −2.5 volts, −0.2 to −2.5 volts, −0.5 to −2.5 volts, −1.0 to −2.5 volts, −1.5 to −2.5 volts, −1.0 to −2.0 volts, −0.5 to −1.0 volts, −0.2 to −1.5 volts, or −2.0 to −2.5 volts.

The voltage between two layers of a device or surface may be varied. In some instances, a voltage is between the anode and cathode. In some instances the voltage is 0.5−3, 1−3, 1.5−2.5, 1−2.5, or 1.5−2 volts. In some instances, the voltage is at least 0.5, 0.75, 1, 1.2, 1.5, 1.7, 1.9, 2, 2.2, 2.4, or more than 2.4 volts. In some instances, the voltage is about 0.5, 0.75, 1, 1.2, 1.5, 1.7, 1.9, 2, 2.2, 2.4, or about 2.4 volts. In some instances, the voltage is −0.1 to −2.5 volts, −0.2 to −2.5 volts, −0.5 to −2.5 volts, −1.0 to −2.5 volts, −1.5 to −2.5 volts, −1.0 to −2.0 volts, −0.5 to −1.0 volts, −0.2 to −1.5 volts, or −2.0 to −2.5 volts. In some instances, a conducting layer of a device is charged with a positive voltage. In some instances, a conducting layer of a device is charged with a negative voltage. In some instances, a first layer of a device is charged with a positive voltage, and a second layer is charged with a negative voltage at the same time.

The (total) amount of time a voltage is applied may be varied for each synthesis cycle (e.g., deblocking, coupling, etc.). Voltage is applied in some instances for no more than 0.1, 0.2, 0.5, 0.8, 1, 2, 5, or no more than 10 seconds. Voltage is applied in some instances for 0.1-10, 0.5-10, 0.5-5, 0.1-5, 2-5, 2-10, 3-10, or 0.1-2 seconds. Voltage is applied in some instances about 0.1, 0.2, 0.5, 0.8, 1, 2, 5, or about 10 seconds. Voltage is applied in some instances for no more than 0.1, 0.2, 0.5, 0.8. 1, 2, 5, 10, 20, 50, 100, 200, 500, 800, or no more than 1000 milliseconds (ms). Voltage is applied in some instances for about 0.1, 0.2, 0.5, 0.8. 1, 2, 5, 10, 20, 50, 100, 200, 500, 800, or about 1000 milliseconds. Voltage is applied in some instances for 0.1-1000, 0.5-500, 0.5-50, 0.1-5, 2-50, 2-100, 3-200, 0.1-10, 1-100, 1-50, or 0.1-2 milliseconds.

Voltage may be applied as a single “on”/“off” cycle, or applied as a series of alternating “on” and “off” cycles to an active device. In some instances an “on” state is a positive voltage or a negative voltage. The application of voltage in the “on” state followed by an “off” state is in some instances defined as a “pulse.” In some instances, voltage is applied in a series of pulses, such as no more than 1, 2, 3, 4, 5, 6, 7, 8, 10, 20, 50, 80, 100, 110, 120, 150, 180, 200, 220, 250, 300, 500, or more than 500 pulses. In some instances, voltage is applied in a series of pulses, such as at least 1, 2, 3, 4, 5, 6, 7, 8, 10, 20, 50, 80, 100, 110, 120, 150, 180, 200, 220, 250, 300, 500, or at least 500 pulses. In some instances, voltage is applied in a series of pulses, such as 1-1000, 1-500, 1-300, 10-500, 10-100, 50-500, 50-200, 100-1000, 2-10, 2-8, 20-200, or 300-750 pulses. In some instances, voltage is applied in a series of pulses, such as about 1, 2, 3, 4, 5, 6, 7, 8, 10, 20, 50, 80, 100, 110, 120, 150, 180, 200, 220, 250, 300, 500, or about 500 pulses. In some instances, voltage is applied in a series of pulses, such as no more than 100, 200, 500, 800, 1000, 2000, 5000, 8000, 10000, 11000, 12000, 15000, 18000, 20000, 50000, 80000, 100,000, 200,000, 500,000, 800,000, or more than 1,000,000 pulses. In some instances, voltage is applied in a series of pulses, such as at least 100, 200, 500, 800, 1000, 2000, 5000, 8000, 10000, 11000, 12000, 15000, 18000, 20000, 50000, 80000, 100,000, 200,000, 500,000, 800,000, or at least 1,000,000 pulses. In some instances, voltage is applied in a series of pulses, such as about 100, 200, 500, 800, 1000, 2000, 5000, 8000, 10000, 11000, 12000, 15000, 18000, 20000, 50000, 80000, 100,000, 200,000, 500,000, 800,000, or about 1,000,000 pulses. In some instances, voltage is applied in a series of pulses, such as at least 10-1000, 10-5000, 100-10,000, 1000-50,000, 10000-100,000, 50000-500,000, 50000-1,000,000, 10,000-100,000 or 500,000-1,000,000 pulses.

The voltage application time may be divided by the number of pulses to define a pulse time (or pulse width, or time per pulse). In some instances a pulse time is no more than 0.1, 0.2, 0.5, 0.8. 1, 2, 5, 10, 20, 50, 100, 200, 500, 800, or no more than 1000 milliseconds. In some instances, a pulse time is 0 seconds. In some instances, a pulse time is greater than 0 seconds. In some instances a pulse time is no more than 0.1, 0.2, 0.5, 0.8. 1, 2, 5, 10, 20, 50, 100, 200, 500, 800, 1000, 2000, or no more than 5000 seconds. In some instances a pulse time is about 0.1, 0.2, 0.5, 0.8. 1, 2, 5, 10, 20, 50, 100, 200, 500, 800, or about 1000 milliseconds. In some instances a pulse time is about 0.1, 0.2, 0.5, 0.8. 1, 2, 5, 10, 20, 50, 100, 200, 500, 800, 1000, 2000, or no more than 5000 seconds. The pulse time in some instances is 0.1-1000, 0.5-500, 0.5-50, 0.1-5, 2-50, 2-100, 3-200, 0.1-10, 1-100, 1-50, or 0.1-2 milliseconds. The pulse time in some instances is 0.1-5000, 0.5-5000, 0.5-2000, 0.1-1000, 2-500, 2-100, 3-200, 0.1-10, 1-1000, 1-500, or 0.1-200 seconds. In some instances a pulse time is no more than 0.1, 0.2, 0.5, 0.8. 1, 2, 5, 10, 20, 50, 100, 200, 500, 800, or no more than 1000 microseconds. In some instances a pulse time is about 0.1, 0.2, 0.5, 0.8. 1, 2, 5, 10, 20, 50, 100, 200, 500, 800, or about 1000 microseconds. The pulse time in some instances is 0.1-1000, 0.5-500, 0.5-50, 0.1-5, 2-50, 2-100, 3-200, 0.1-10, 1-100, 1-50, or 0.1-2 microseconds. In some instances, a polynucleotide synthesis surface is washed with a solvent in between pulses. In some instances, a polynucleotide synthesis surface is not washed with a solvent in between pulses. In some instances a series of pulses are used to deliver voltage to a surface, followed by a wash step, followed by another series of pulses. Pulses need not be the same voltage. In some instances, a first pulse is positive, and a second pulse is negative. In some instances, the time between a positive and negative voltage is substantially instantaneous. In some instances, a first pulse is about 2 volts and a second pulse is about −0.6 volts. In some instances, a first pulse is 0.5 to 3 volts and a second pulse is −0.1 to −1.0 volts.

The time period between pulses may be varied to allow, without being bound by theory, electrochemically generated reagents to dissipate. The time between pulses in some instances is no more than 0.1, 0.2, 0.5, 0.8, 1, 2, 5, or no more than 10 seconds. The time between pulses in some instances is 0.1-10, 0.5-10, 0.5-5, 0.1-5, 2-5, 2-10, 3-10, or 0.1-2 seconds. The time between pulses in some instances is about 0.1, 0.2, 0.5, 0.8, 1, 2, 5, or about 10 seconds. The time between pulses in some instances is no more than 0.1, 0.2, 0.5, 0.8. 1, 2, 5, 10, 20, 50, 100, 200, 500, 800, or no more than 1000 milliseconds (ms). The time between pulses in some instances is about 0.1, 0.2, 0.5, 0.8. 1, 2, 5, 10, 20, 50, 100, 200, 500, 800, or about 1000 milliseconds. The time between pulses in some instances is 0.1-1000, 0.5-500, 0.5-50, 0.1-5, 2-50, 2-100, 3-200, 0.1-10, 1-100, 1-50, or 0.1-2 milliseconds. The time between pulses in some instances is no more than 0.1, 0.2, 0.5, 0.8. 1, 2, 5, 10, 20, 50, 100, 200, 500, 800, or no more than 1000 microseconds (ms). The time between pulses in some instances is about 0.1, 0.2, 0.5, 0.8. 1, 2, 5, 10, 20, 50, 100, 200, 500, 800, or about 1000 microseconds. The time between pulses in some instances is 0.1-1000, 0.5-500, 0.5-50, 0.1-5, 2-50, 2-100, 3-200, 0.1-10, 1-100, 1-50, or 0.1-2 microseconds. In some instances, the ratio between on and off times for series of pulses is described as a duty cycle. In some instances, a duty cycle is about 1:100, 1:50, 1:20, 1:10, 1:5, 1:2, 1:1.5, 1:1.05, 1.05:1, 1.5:1, 2:1, or about 3:1. In some instances, a duty cycle is no more than 1:100, 1:50, 1:20, 1:10, 1:5, 1:2, 1:1.5, 1:1.05, 1.05:1, 1.5:1, 2:1, or no more than 3:1. In some instances, a duty cycle is at least 1:100, 1:50, 1:20, 1:10, 1:5, 1:2, 1:1.5, 1:1.05, 1.05:1, 1.5:1, 2:1, or at least 3:1.

Various chemical reactions may be used to deblock polynucleotides, directly or indirectly. In some instances electrical voltage oxidizes or reduces a blocking group (protecting group) directly on a polynucleotide, causing the polynucleotide to be deblocked. In some instances, the voltage generates an in-situ reagent which deblocks the blocked nucleotide. In some instances, the polynucleotide comprises an acid-cleavable blocking group. In some instances, the reagent is dissolved in a solvent. In some instances, the reagent is an acid, such as H⁺. Various reagents may be used to electrochemically generate deblocking reagents such as acids. In some instances, a reagent comprises a quinone. In some instances, the quinone is benzoquinone, hydroquinone, anthraquinone, substituted benzoquinone, a hydrazine, a diazirine, or other reagent configured to generate an acid when a voltage is applied. In some instances, the reagent comprises a mixture of hydroquinone (HQ) and benzoquinone (BQ). In some instances, the reagent comprises a mixture of hydroquinone and benzoquinone, wherein the ratio of HQ:BQ is about 100:1, 50:1, 20:1, 10:1, 8:1, 5:1, 3:1, 2:1, 1:1, 1:2, 1:5, or about 1:10. In some instances, the ratio of HQ:BQ is at least 100:1, 50:1, 20:1, 10:1, 8:1, 5:1, 3:1, 2:1, or at least 1:1. In some instances, the ratio of HQ:BQ is no more than 100:1, 50:1, 20:1, 10:1, 8:1, 5:1, 3:1, 2:1, or no more than 1:1. In some instances, the ratio of HQ:BQ is 100:1-10:1, 50:1-1:1, 20:1-5:1, 15:1-5:1, 10:1-1:1, 10:1-2:1. 20:1-2:1, 1:1-1:5, 1:1-10:1 10:1-1:10, or 5:1-1:5.

Provided herein are methods of fabricating the devices and surfaces for polynucleotide synthesis. Described herein are layers integrated into a solid support. In some instances, layers comprise electrodes or are configured for use as electrodes. In some instances, electrodes are configured as cathodes or anodes. In some instances, an anode comprises a metal oxide. In some instances, nucleic acids are synthesized on an anode. In some instances, nucleic acids are synthesized on a metal oxide layer. In some instances, nucleic acids are synthesized on a porous metal oxide layer comprising a continuous metal layer beneath it. Electrodes in some instances comprise at least one conductor, and are fabricated of materials well known in the art. In some instances, electrodes comprise at least one conductor and one or more insulators or semi-conductors. Materials may comprise metals, non-metals, mixed-metal oxides, nitrides, carbides, silicon-based materials, or other material. In some instances, metal oxides include TiO₂, Ta₂O₅, IrO₂, RuO₂, RhO₂, Nb₂O₅, A₁₂O₃, BaO, Y₂O₃, HfO₂, SrO or other metal oxide known in the art. In some instances, metal carbides include TiC, WC, ThC₂, ThC, VC, W₂C, ZrC, HfC, NbC, TaC, Ta₂C, or other metal carbide known in the art. In some instances, metal nitrides include GaN, InN, BN, Be₃N₂, Cr₂N, MoN, Si₃N₄, TaN, Th₂N₂, VN, ZrN, TiN, HfN, NbC, WN, TaN, or other metal nitride known in the art. In some instances, a device disclosed herein is manufactured with a combination of materials listed herein or any other suitable material known in the art.

Solid supports comprising layers may be coated with additional materials such as semiconductors or insulators. In some instances, a layer is configured for use as an electrode. In some instances, electrodes are coated with materials for polynucleotide attachment and synthesis. Each electrode can control one, or a plurality of different loci for synthesis, wherein each locus for synthesis has a density of polynucleotides. In some instances, the density is at least 1 oligo per 10 nm², 20, 50, 100, 200, 500, 1,000, 2,000, 5,000 or at least 1 oligo per 10,000 nm². In some instances, the density is about 1 oligo per 10 nm² to about 1 oligo per 5,000 nm², about 1 oligo per 50 nm² to about 1 oligo per 500 nm², or about 1 oligo per 25 nm² to about 1 oligo per 75 nm². In some instances, the density of polynucleotides is about 1 oligo per 25 nm² to about 1 oligo per 75 nm².

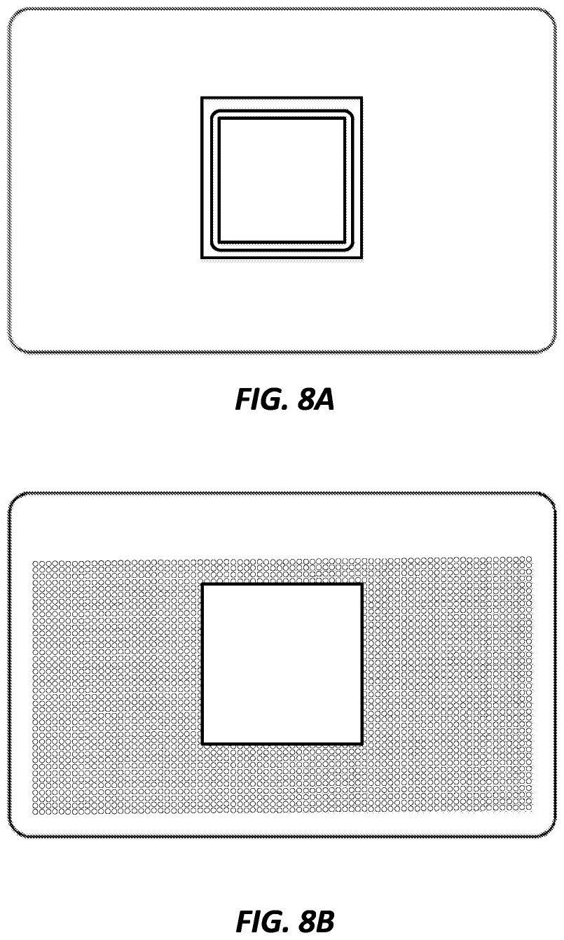

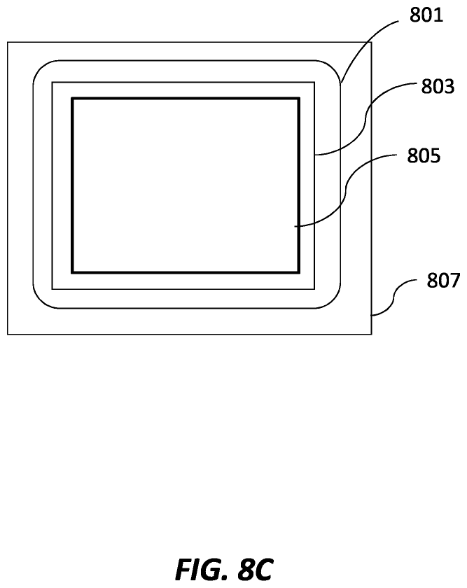

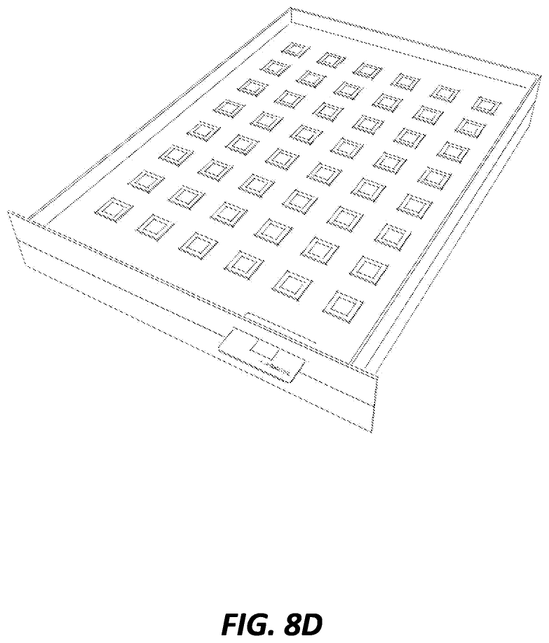

Described herein are devices wherein two or more solid supports are assembled. In some instances, solid supports are interfaced together on a larger unit. Interfacing may comprise exchange of fluids, electrical signals, or other medium of exchange between solid supports. This unit is capable of interface with any number of servers, computers, or networked devices. For example, a plurality of solid support is integrated onto a rack unit, which is conveniently inserted or removed from a server rack. The rack unit may comprise any number of solid supports. In some instances the rack unit comprises at least 1, 2, 5, 10, 20, 50, 100, 200, 500, 1000, 2000, 5000, 10,000, 20,000, 50,000, 100,000 or more than 100,000 solid supports. In some instances, two or more solid supports are not interfaced with each other. Nucleic acids (and the information stored in them) present on solid supports can be accessed from the rack unit. See, e.g., FIG. 8D. In some instances, solid supports are present on solid supports such as chips (FIGS. 8A-8C). Access includes removal of polynucleotides from solid supports, direct analysis of polynucleotides on the solid support, or any other method which allows the information stored in the nucleic acids to be manipulated or identified. Information in some instances is accessed from a plurality of racks, a single rack, a single solid support in a rack, a portion of the solid support, or a single locus on a solid support. In various instances, access comprises interfacing nucleic acids with additional devices such as mass spectrometers, HPLC, sequencing instruments, PCR thermocyclers, or other device for manipulating nucleic acids. Access to nucleic acid information in some instances is achieved by cleavage of polynucleotides from all or a portion of a solid support. Cleavage in some instances comprises exposure to chemical reagents (ammonia or other reagent), electrical potential, radiation, heat, light, acoustics, or other form of energy capable of manipulating chemical bonds. In some instances, cleavage occurs by charging one or more electrodes in the vicinity of the polynucleotides. In some instances, electromagnetic radiation in the form of UV light is used for cleavage of polynucleotides. In some instances, a lamp is used for cleavage of polynucleotides, and a mask mediates exposure locations of the UV light to the surface. In some instances, a laser is used for cleavage of polynucleotides, and a shutter opened/closed state controls exposure of the UV light to the surface. In some instances, access to nucleic acid information (including removal/addition of racks, solid supports, reagents, nucleic acids, or other component) is completely automated.

Solid supports as described herein comprise an active area. In some instances, the active area comprises addressable solid supports, regions, or loci for nucleic acid synthesis. In some instances, the active area comprises addressable regions or loci for nucleic acid storage. In some instances, an active area is in fluid communication with solvents or other reagents. The active area comprises varying dimensions. For example, the dimension of the active area is between about 1 mm to about 50 mm by about 1 mm to about 50 mm. In some instances, the active area comprises a width of at least or about 0.5, 1, 1.5, 2, 2.5, 3, 5, 5, 10, 12, 14, 16, 18, 20, 25, 30, 35, 40, 45, 50, 60, 70, 80, or more than 80 mm. In some instances, the active area comprises a height of at least or about 0.5, 1, 1.5, 2, 2.5, 3, 5, 5, 10, 12, 14, 16, 18, 20, 25, 30, 35, 40, 45, 50, 60, 70, 80, or more than 80 mm. An exemplary active area within a solid support is seen in FIG. 8C. A package 807 comprises an active area 805 within a solid support 803. The package 807 also comprises a fluidics interface 801.

Described herein are devices, compositions, systems and methods for solid support based nucleic acid synthesis and storage, wherein the solid support has a number of sites (e.g., spots) or positions for synthesis or storage. In some instances, the solid support comprises up to or about 10,000 by 10,000 positions in an area. In some instances, the solid support comprises between about 1000 and 20,000 by between about 1000 and 20,000 positions in an area. In some instances, the solid support comprises at least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions by least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions in an area. In some instances the area is up to 0.25, 0.5, 0.75, 1.0, 1.25, 1.5, or 2.0 inches squared. In some instances, the solid support comprises addressable loci having a pitch of at least or about 0.1, 0.2, 0.25, 0.3, 0.4, 0.5, 1.0, 1.5, 2.0, 2.5, 3.0, 3.5, 4.0, 4.5, 5, 6, 7, 8, 9, 10, or more than 10 um. In some instances, the solid support comprises addressable loci having a pitch of about 5 um. In some instances, the solid support comprises addressable loci having a pitch of about 2 um. In some instances, the solid support comprises addressable loci having a pitch of about 1 um. In some instances, the solid support comprises addressable loci having a pitch of about 0.2 um. In some instances, the solid support comprises addressable loci having a pitch of about 0.2 um to about 10 um, about 0.2 to about 8 um, about 0.5 to about 10 um, about 1 um to about 10 um, about 2 um to about 8 um, about 3 um to about 5 um, about 1 um to about 3 um or about 0.5 um to about 3 um. In some instances, the solid support comprises addressable loci having a pitch of about 0.1 um to about 3 um. In some instances, the solid support comprises addressable loci having a pitch of at least or about 0.01, 0.02, 0.025, 0.03, 0.04, 0.05, 0.1, 0.15, 0.02, 0.25, 0.30, 0.35, 0.4, 0.45, 0.5, 0.6, 0.7, 0.8, 0.9, 1, or more than 1 um. In some instances, the solid support comprises addressable loci having a pitch of about 0.5 um. In some instances, the solid support comprises addressable loci having a pitch of about 0.2 um. In some instances, the solid support comprises addressable loci having a pitch of about 0.1 um. In some instances, the solid support comprises addressable loci having a pitch of about 0.02 um. In some instances, the solid support comprises addressable loci having a pitch of about 0.02 um to about 1 um, about 0.02 to about 0.8 um, about 0.05 to about 0.1 um, about 0.1 um to about 1 um, about 0.2 um to about 0.8 um, about 0.3 um to about 0.5 um, about 0.1 um to about 0.3 um or about 0.05 um to about 0.3 um. In some instances, the solid support comprises addressable loci having a pitch of about 0.01 um to about 0.3 um.

The solid support for nucleic acid synthesis or storage as described herein comprises a high capacity for storage of data. For example, the capacity of the solid support is at least or about 1, 2, 3, 4, 5, 6, 7, 8, 9, 10, 20, 50, 100, 200, 300, 400, 500, 600, 700, 800, 900, 1000, or more than 1000 petabytes. In some instances, the capacity of the solid support is between about 1 to about 10 petabytes or between about 1 to about 100 petabytes. In some instances, the capacity of the solid support is about 100 petabytes. In some instances, the data is stored as addressable arrays of packets as droplets. In some instances, the data is stored as addressable arrays of packets as droplets on a spot. In some instances, the data is stored as addressable arrays of packets as dry wells. In some instances, the addressable arrays comprise at least or about 1, 2, 3, 4, 5, 6, 7, 8, 9, 10, 20, 50, 100, 200, or more than 200 gigabytes of data. In some instances, the addressable arrays comprise at least or about 1, 2, 3, 4, 5, 6, 7, 8, 9, 10, 20, 50, 100, 200, or more than 200 terabytes of data. In some instances, an item of information is stored in a background of data. For example, an item of information encodes for about 10 to about 100 megabytes of data and is stored in 1 petabyte of background data. In some instances, an item of information encodes for at least or about 1, 10, 20, 30, 40, 50, 60, 70, 80, 90, 100, 150, 200, 300, 400, 500, or more than 500 megabytes of data and is stored in 1, 10, 20, 30, 40, 50, 60, 70, 80, 90, 100, 150, 200, 300, 400, 500, or more than 500 petabytes of background data.

### Structures for Polynucleotide Synthesis

Provided herein are rigid or flexibles structures for polynucleotide synthesis. In the case of rigid structures, provided herein are devices having a structure for the generation of a library of polynucleotides. In some instances, the structure comprises a plate.

In the case of flexible structures, provided herein are devices wherein the flexible structure comprises a continuous loop wrapped around one or more fixed structures, e.g., a pair of rollers or a non-continuous flexible structure wrapped around separate fixed structures, e.g., a pair reels. In some instances, the structures comprise multiple regions for polynucleotide synthesis. An exemplary structure comprises a plate with distinct regions for polynucleotide synthesis. The distinct regions may be separated by breaking or cutting. Each of the distinct regions may be further released, sequenced, decrypted, and read or stored. An alternative structure comprises a tape comprises with distinct regions for polynucleotide synthesis. The distinct regions may be separated by breaking or cutting. Each of the distinct regions may be further released, sequenced, decrypted, and read or stored. Provided herein are flexible structures having a surface with a plurality of loci for polynucleotide extension. Each locus in a portion of the flexible structure, may be a substantially planar spot (e.g., flat), a channel, or a well. In some instances, each locus of the structure has a width of about 10 um and a distance between the center of each structure of about 21 um. In some instances, each locus of the structure has a width of about 1 um and a distance between the center of each structure of about 2 um. In some instances, each locus of the structure has a width of about 0.1 um and a distance between the center of each structure of about 0.2 um. Loci may comprise, without limitation, circular, rectangular, tapered, or rounded shapes. Alternatively or in combination, the structures are rigid. In some instances, the rigid structures comprise loci for polynucleotide synthesis. In some instances, the rigid structures comprise substantially planar regions, channels, or wells for polynucleotide synthesis.

In some instances, a well described herein has a width to depth (or height) ratio of 1 to 0.01, wherein the width is a measurement of the width at the narrowest segment of the well. In some instances, a well described herein has a width to depth (or height) ratio of 0.5 to 0.01, wherein the width is a measurement of the width at the narrowest segment of the well. In some instances, a well described herein has a width to depth (or height) ratio of about 0.01, 0.05, 0.1, 0.15, 0.16, 0.2, 0.5, or 1. Provided herein are structures for polynucleotide synthesis comprising a plurality of discrete loci for polynucleotide synthesis. Exemplary structures for the loci include, without limitation, substantially planar regions, channels, wells or protrusions. Structures described herein are may comprise a plurality of clusters, each cluster comprising a plurality of wells, loci or channels. Alternatively, described herein are may comprise a homogenous arrangement of wells, loci or channels. Structures provided herein may comprise wells having a height or depth from about 5 um to about 500 um, from about 5 um to about 400 um, from about 5 um to about 300 um, from about 5 um to about 200 um, from about 5 um to about 100 um, from about 5 um to about 50 um, or from about 10 um to about 50 um. In some instances, the height of a well is less than 100 um, less than 80 um, less than 60 um, less than 40 um or less than 20 um. In some instances, well height is about 10, 20, 30, 40, 50, 60, 70, 80, 90, 100, 200, 300, 400, 500 um or more. In some instances, the height or depth of the well is at least 10, 25, 50, 75, 100, 200, 300, 400, 500, 600, 700, 800, 900, 1000, or more than 1000 mn. In some instances, the height or depth of the well is in a range of about 10 nm to about 1000 nm, about 25 nm to about 900 nm, about 50 nm to about 800 nm, about 75 nm to about 700 nm, about 100 nm to about 600 nm, or about 200 nm to about 500. In some instances, the height or depth of the well is in a range of about 50 nm to about 1 um. In some instances, well height is about 10, 20, 30, 40, 50, 60, 70, 80, 90, 100, 200, 300, 400, 500, 700, 800, 900 or about 1000 nm.

Structures for polynucleotide synthesis provided herein may comprise channels. The channels may have a width to depth (or height) ratio of 1 to 0.01, wherein the width is a measurement of the width at the narrowest segment of the microchannel. In some instances, a channel described herein has a width to depth (or height) ratio of 0.5 to 0.01, wherein the width is a measurement of the width at the narrowest segment of the microchannel. In some instances, a channel described herein has a width to depth (or height) ratio of about 0.01, 0.05, 0.1, 0.15, 0.16, 0.2, 0.5, or 1.

Described herein are structures for polynucleotide synthesis comprising a plurality of discrete loci. Structures comprise, without limitation, substantially planar regions, channels, protrusions, or wells for polynucleotide synthesis. In some instances, structures described herein are provided comprising a plurality of channels, wherein the height or depth of the channel is from about 5 um to about 500 um, from about 5 um to about 400 um, from about 5 um to about 300 um, from about 5 um to about 200 um, from about 5 um to about 100 um, from about 5 um to about 50 um, or from about 10 um to about 50 um. In some cases, the height of a channel is less than 100 um, less than 80 um, less than 60 um, less than 40 um or less than 20 um. In some cases, channel height is about 10, 20, 30, 40, 50, 60, 70, 80, 90, 100, 200, 300, 400, 500 um or more. In some instances, the height or depth of the channel is at least 10, 25, 50, 75, 100, 200, 300, 400, 500, 600, 700, 800, 900, 1000, or more than 1000 nm. In some instances, the height or depth of the channel is in a range of about 10 nm to about 1000 nm, about 25 nm to about 900 nm, about 50 nm to about 800 nm, about 75 nm to about 700 nm, about 100 nm to about 600 nm, or about 200 nm to about 500. Channels described herein may be arranged on a surface in clusters or as a homogenous field.

The width of a locus on the surface of a structure for polynucleotide synthesis described herein may be from about 0.1 um to about 500 um, from about 0.5 um to about 500 um, from about 1 um to about 200 um, from about 1 um to about 100 um, from about 5 um to about 100 um, or from about 0.1 um to about 100 um, for example, about 90 um, 80 um, 70 um, 60 um, 50 um, 40 um, 30 um, 20 um, 10 um, 5 um, 1 um or 0.5 um. In some instances, the width of a locus is less than about 100 um, 90 um, 80 um, 70 um, 60 um, 50 um, 40 um, 30 um, 20 um or 10 um. In some instances, the width of a locus is at least 10, 25, 50, 75, 100, 200, 300, 400, 500, 600, 700, 800, 900, 1000, or more than 1000 nm. In some instances, the width of a locus is in a range of about 10 nm to about 1000 nm, about 25 nin to about 900 nm, about 50 nrn to about 800 nm, about 75 nm to about 700 nm, about 100 nm to about 600 nm, or about 200 nm to about 500. In some instances, the width of a locus is in a range of about 50 nm to about 1000 nm. In some instances, the distance between the center of two adjacent loci is from about 0.1 um to about 500 um, 0.5 um to about 500 um, from about 1 um to about 200 um, from about 1 um to about 100 um, from about 5 um to about 200 um, from about 5 um to about 100 um, from about 5 um to about 50 um, or from about 5 um to about 30 um, for example, about 20 um. In some instances, the total width of a locus is about 5 um, 10 um, 20 um, 30 um, 40 um, 50 um, 60 um, 70 um, 80 um, 90 um, or 100 um. In some instances, the total width of a locus is about 1 um to 100 um, 30 um to 100 um, or 50 um to 70 um. In some instances, the distance between the center of two adjacent loci is from about 0.5 um to about 2 um, 0.5 um to about 2 um, from about 0.75 um to about 2 um, from about 1 um to about 2 um, from about 0.2 um to about 1 um, from about 0.5 um to about 1.5 um, from about 0.5 um to about 0.8 um, or from about 0.5 um to about 1 um, for example, about 1 um. In some instances, the total width of a locus is about 50 nm, 0.1 um, 0.2 um, 0.3 um, 0.4 um, 0.5 um, 0.6 um, 0.7 um, 0.8 um, 0.9 um, 1 um, 1.1 um, 1.2 um, 1.3 um, 1.4 um, or 1.5 um. In some instances, the total width of a locus is about 0.5 um to 2 um, 0.75 um to 1 um, or 0.9 um to 2 um.

In some instances, each locus supports the synthesis of a population of polynucleotides having a different sequence than a population of polynucleotides grown on another locus. Provided herein are surfaces which comprise at least 10, 100, 256, 500, 1000, 2000, 3000, 4000, 5000, 6000, 7000, 8000, 9000, 10000, 11000, 12000, 13000, 14000, 15000, 20000, 30000, 40000, 50000 or more clusters. Provided herein are surfaces which comprise more than 2,000; 5,000; 10,000; 20,000; 30,000; 50,000; 100,000; 200,000; 300,000; 400,000; 500,000; 600,000; 700,000; 800,000; 900,000; 1,000,000; 5,000,000; or 10,000,000 or more distinct loci. In some cases, each cluster includes 1, 2, 3, 4, 5, 6, 7, 8, 9, 10, 20, 30, 40, 50, 60, 70, 80, 90, 100, 120, 130, 150, 200, 500 or more loci. In some cases, each cluster includes 50 to 500, 50 to 200, 50 to 150, or 100 to 150 loci. In some cases, each cluster includes 100 to 150 loci. In some instances, each cluster includes 109, 121, 130 or 137 loci.

Provided herein are loci having a width at the longest segment of 5 to 100 um. In some cases, the loci have a width at the longest segment of about 30, 35, 40, 45, 50, 55 or 60 um. In some cases, the loci are channels having multiple segments, wherein each segment has a center to center distance apart of 5 to 50 um. In some cases, the center to center distance apart for each segment is about 5, 10, 15, 20 or 25 um.

Provided herein are loci having a width at the longest segment of 5 to 500 nm. In some cases, the loci have a width at the longest segment of about 30, 35, 40, 45, 50, 55, 60, 80, or 100 nm. In some cases, the loci are channels having multiple segments, wherein each segment has a center to center distance apart of 5 to 50 nm. In some cases, the center to center distance apart for each segment is about 5, 10, 15, 20, 25, 50, 100, or 200 nm.

In some instances, the number of distinct polynucleotides synthesized on the surface of a structure described herein is dependent on the number of distinct loci available in the substrate. In some instances, the density of loci within a cluster of a substrate is at least or about 1 locus per mm², 10 loci per mm², 25 loci per mm², 50 loci per mm², 65 loci per mm², 75 loci per mm², 100 loci per mm², 130 loci per mm², 150 loci per mm², 175 loci per mm², 200 loci per mm², 300 loci per mm², 400 loci per mm², 500 loci per mm², 1,000 loci per mm², 10⁴ loci per mm², 10⁵ loci per mm², 10⁶ loci per mm², or more. In some cases, a substrate comprises from about 10 loci per mm² to about 500 mm², from about 25 loci per mm² to about 400 mm², from about 50 loci per mm² to about 500 mm², from about 100 loci per mm² to about 500 mm², from about 150 loci per mm² to about 500 mm², from about 10 loci per mm² to about 250 mm², from about 50 loci per mm² to about 250 mm², from about 10 loci per mm² to about 200 mm², or from about 50 loci per mm² to about 200 mm². In some cases, a substrate comprises from about 104 loci per mm² to about 10⁵ mm². In some cases, a substrate comprises from about 10⁵ loci per mm² to about 10⁷ mm². In some cases, a substrate comprises at least 10⁵ loci per mm². In some cases, a substrate comprises at least 10⁶ loci per mm². In some cases, a substrate comprises at least 10?loci per mm². In some cases, a substrate comprises from about 10 loci per mm² to about 10⁵ mm². In some instances, the density of loci within a cluster of a substrate is at least or about 1 locus per um², 10 loci per um², 25 loci per um², 50 loci per um², 65 loci per um², 75 loci per um², 100 loci per um², 130 loci per um², 150 loci per um², 175 loci per um², 200 loci per um², 300 loci per um², 400 loci per um², 500 loci per um², 1,000 loci per um² or more. In some cases, a substrate comprises from about 10 loci per um² to about 500 um², from about 25 loci per um² to about 400 um², from about 50 loci per um² to about 500 um², from about 100 loci per um² to about 500 um², from about 150 loci per um² to about 500 um², from about 10 loci per um² to about 250 um², from about 50 loci per um² to about 250 um², from about 10 loci per um² to about 200 um², or from about 50 loci per um² to about 200 um².

In some instances, the distance between the centers of two adjacent loci within a cluster is from about 10 um to about 500 um, from about 10 um to about 200 um, or from about 10 um to about 100 um. In some cases, the distance between two centers of adjacent loci is greater than about 10 um, 20 um, 30 um, 40 um, 50 um, 60 um, 70 um, 80 um, 90 um or 100 um. In some cases, the distance between the centers of two adjacent loci is less than about 200 um, 150 um, 100 um, 80 um, 70 um, 60 um, 50 um, 40 um, 30 um, 20 um or 10 um. In some cases, the distance between the centers of two adjacent loci is less than about 10000 nm, 8000 nm, 6000 nm, 4000 nm, 2000 nm 1000 nm, 800 nm, 600 nm, 400 run, 200 nm, 150 nm, 100 nm, 80 um, 70 nm, 60 nm, 50 nm, 40 nm, 30 nm, 20 mn or 10 nm. In some instances, each square meter of a structure described herein allows for at least 10⁷, 10⁸, 10⁹, 10¹⁰, 10¹¹ loci, where each locus supports one polynucleotide. In some instances, 10⁹ polynucleotides are supported on less than about 6, 5, 4, 3, 2 or 1 m² of a structure described herein.

In some instances, a structure described herein provides support for the synthesis of more than 2,000; 5,000; 10,000; 20,000; 30,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some cases, the structure provides support for the synthesis of more than 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence. In some instances, the structure provides a surface environment for the growth of polynucleotides having at least 50, 60, 70, 75, 80, 85, 90, 95, 100, 110, 120, 130, 140, 150, 160, 175, 200, 225, 250, 275, 300, 325, 350, 375, 400, 425, 450, 475, 500 bases or more. In some arrangements, structures for polynucleotide synthesis described herein comprise sites for polynucleotide synthesis in a uniform arrangement.

In some instances, polynucleotides are synthesized on distinct loci of a structure, wherein each locus supports the synthesis of a population of polynucleotides. In some cases, each locus supports the synthesis of a population of polynucleotides having a different sequence than a population of polynucleotides grown on another locus. In some instances, the loci of a structure are located within a plurality of clusters. In some instances, a structure comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a structure comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some cases, each cluster includes 1, 2, 3, 4, 5, 6, 7, 8, 9, 10, 20, 30, 40, 50, 60, 70, 80, 90, 100, 120, 130, 150 or more loci. In some instances, each cluster includes 50 to 500, 100 to 150, or 100 to 200 loci. In some instances, each cluster includes 109, 121, 130 or 137 loci. In some instances, each cluster includes 5, 6, 7, 8, 9, 10, 11 or 12 loci. In some instances, polynucleotides from distinct loci within one cluster have sequences that, when assembled, encode for a contiguous longer polynucleotide of a predetermined sequence.

### Structure Size

In some instances, a structure described herein is about the size of a plate (e.g., chip), for example between about 40 and 120 mm by between about 25 and 100 mm. In some instances, a structure described herein has a diameter less than or equal to about 1000 mm, 500 mm, 450 mm, 400 mm, 300 mm, 250 nm, 200 mm, 150 mm, 100 mm or 50 mm. In some instances, the diameter of a substrate is between about 25 mm and 1000 mm, between about 25 mm and about 800 mm, between about 25 mm and about 600 mm, between about 25 mm and about 500 mm, between about 25 mm and about 400 mm, between about 25 mm and about 300 mm, or between about 25 mm and about 200. Non-limiting examples of substrate size include about 300 mm, 200 mm, 150 mm, 130 mm, 100 mm, 84 mm, 76 mm, 54 mm, 51 mm and 25 mm. In some instances, a substrate has a planar surface area of at least 100 mm²; 200 mm²; 500 mm²; 1,000 mm²; 2,000 mm²; 4,500 mm²; 5,000 mm²; 10,000 mm²; 12,000 mm²; 15,000 mm²; 20,000 mm²; 30,000 mm²; 40,000 mm²; 50,000 mm² or more. In some instances, the thickness is between about 50 mm and about 2000 mm, between about 50 mm and about 1000 mm, between about 100 mm and about 1000 mm, between about 200 mm and about 1000 mm, or between about 250 mm and about 1000 mm. Non-limiting examples thickness include 275 mm, 375 mm, 525 mm, 625 mm, 675 mm, 725 mm, 775 mm and 925 mm. In some instances, the thickness is at least or about 0.5 mm, 1.0 mm, 1.5 mm, 2.0 mm, 2.5 mm, 3.0 mm, 3.5 mm, 4.0 mm, or more than 4.0 mm. In some cases, the thickness of varies with diameter and depends on the composition of the substrate. For example, a structure comprising materials other than silicon may have a different thickness than a silicon structure of the same diameter. Structure thickness may be determined by the mechanical strength of the material used and the structure must be thick enough to support its own weight without cracking during handling. In some instances, a structure is more than about 1, 2, 3, 4, 5, 10, 15, 30, 40, 50 feet in any one dimension.

### Materials

Provided herein are devices comprising a surface, wherein the surface is modified to support polynucleotide synthesis at predetermined locations and with a resulting low error rate, a low dropout rate, a high yield, and a high oligo representation. In some instances, surfaces of devices for polynucleotide synthesis provided herein are fabricated from a variety of materials capable of modification to support a de novo polynucleotide synthesis reaction. In some cases, the devices are sufficiently conductive, e.g., are able to form uniform electric fields across all or a portion of the devices. Devices described herein may comprise a flexible material. Exemplary flexible materials include, without limitation, modified nylon, unmodified nylon, nitrocellulose, and polypropylene. Devices described herein may comprise a rigid material. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, gold, platinum). Devices disclosed herein may be fabricated from a material comprising silicon, polystyrene, agarose, dextran, cellulosic polymers, polyacrylamides, polydimethylsiloxane (PDMS), glass, or any combination thereof. In some cases, devices disclosed herein are manufactured with a combination of materials listed herein or any other suitable material known in the art.

Devices described herein may comprise material having a range of tensile strength. Exemplary materials having a range of tensile strengths include, but are not limited to, nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.8 MPa). Solid supports described herein can have a tensile strength from 1 to 300, 1 to 40, 1 to 10, 1 to 5, or 3 to 11 MPa. Solid supports described herein can have a tensile strength of about 1, 1.5, 2, 3, 4, 5, 6, 7, 8, 9, 10, 11, 20, 25, 40, 50, 60, 70, 80, 90, 100, 150, 200, 250, 270, or more MPa. In some instances, a device described herein comprises a solid support for polynucleotide synthesis that is in the form of a flexible material capable of being stored in a continuous loop or reel, such as a tape or flexible sheet.

Young's modulus measures the resistance of a material to elastic (recoverable) deformation under load. Exemplary materials having a range of Young's modulus stiffness include, but are not limited to, nylon (3 GPa), nitrocellulose (1.5 GPa), polypropylene (2 GPa), silicon (150 GPa), polystyrene (3 GPa), agarose (1-10 GPa), polyacrylamide (1-10 GPa), polydimethylsiloxane (PDMS) (1-10 GPa). Solid supports described herein can have a Young's moduli from 1 to 500, 1 to 40, 1 to 10, 1 to 5, or 3 to 11 GPa. Solid supports described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load. In some instances, a solid support described herein has a surface with a flexibility of at least nylon.

In some cases, devices disclosed herein comprise a silicon dioxide base and a surface layer of silicon oxide. Alternatively, the devices may have a base of silicon oxide. Surface of the devices provided here may be textured, resulting in an increase overall surface area for polynucleotide synthesis. Devices disclosed herein in some instances comprise at least 5%, 10%, 25%, 50%, 80%, 90%, 95%, or 99% silicon. Devices disclosed herein in some instances are fabricated from silicon on insulator (SOI) wafer.

The structure may be fabricated from a variety of materials, suitable for the methods and compositions of the invention described herein. In instances, the materials from which the substrates/solid supports of the comprising the invention are fabricated exhibit a low level of polynucleotide binding. In some situations, material that are transparent to visible and/or UV light can be employed. Materials that are sufficiently conductive, e.g. those that can form uniform electric fields across all or a portion of the substrates/solids support described herein, can be utilized. In some instances, such materials may be connected to an electric ground. In some cases, the substrate or solid support can be heat conductive or insulated. The materials can be chemical resistant and heat resistant to support chemical or biochemical reactions such as a series of polynucleotide synthesis reactions. For flexible materials, materials of interest include nylon, both modified and unmodified, nitrocellulose, polypropylene, and the like.

For rigid materials, specific materials of interest include glass; fuse silica; silicon, plastics (for example polytetrafluoroethylene, polypropylene, polystyrene, polycarbonate, and blends thereof, and the like); metals (for example, gold, platinum, and the like). The structure can be fabricated from a material selected from the group consisting of silicon, polystyrene, agarose, dextran, cellulosic polymers, poly acrylamides, polydimethylsiloxane (PDMS), and glass. The substrates/solid supports or the microstructures, reactors therein may be manufactured with a combination of materials listed herein or any other suitable material known in the art.

In some instances, a substrate disclosed herein comprises a computer readable material. Computer readable materials include, without limitation, magnetic media, reel-to-reel tape, cartridge tape, cassette tape, flexible disk, paper media, film, microfiche, continuous tape (e.g., a belt) and any media suitable for storing electronic instructions. In some cases, the substrate comprises magnetic reel-to-reel tape or a magnetic belt. In some instances, the substrate comprises a flexible printed circuit board. Structures described herein may be transparent to visible and/or UV light. In some instances, structures described herein are sufficiently conductive to form uniform electric fields across all or a portion of a structure. In some instances, structures described herein are heat conductive or insulated. In some instances, the structures are chemical resistant and heat resistant to support a chemical reaction such as a polynucleotide synthesis reaction. In some instances, the substrate is magnetic. In some instances, the structures comprise a metal or a metal alloy.

Structures for polynucleotide synthesis may be over 1, 2, 5, 10, 30, 50 or more feet long in any dimension. In the case of a flexible structure, the flexible structure is optionally stored in a wound state, e.g., in a reel. In the case of a large rigid structure, e.g., greater than 1 foot in length, the rigid structure can be stored vertically or horizontally.

### Surface Preparation

Provided herein are methods to support the immobilization of a biomolecule on a substrate, where a surface of a structure described herein comprises a material and/or is coated with a material that facilitates a coupling reaction with the biomolecule for attachment. To prepare a structure for biomolecule immobilization, surface modifications may be employed that chemically and/or physically alter the substrate surface by an additive or subtractive process to change one or more chemical and/or physical properties of a substrate surface or a selected site or region of the surface. For example, surface modification involves (1) changing the wetting properties of a surface, (2) functionalizing a surface, i.e. providing, modifying or substituting surface functional groups, (3) defunctionalizing a surface, i.e. removing surface functional groups, (4) otherwise altering the chemical composition of a surface, e.g., through etching, (5) increasing or decreasing surface roughness. (6) providing a coating on a surface, e.g., a coating that exhibits wetting properties that are different from the wetting properties of the surface, and/or (7) depositing particulates on a surface. In some instances, the surface of a structure is selectively functionalized to produce two or more distinct areas on a structure, wherein at least one area has a different surface or chemical property that another area of the same structure. Such properties include, without limitation, surface energy, chemical termination, surface concentration of a chemical moiety, and the like.

In some instances, a surface of a structure disclosed herein is modified to comprise one or more actively functionalized surfaces configured to bind to both the surface of the substrate and a biomolecule, thereby supporting a coupling reaction to the surface. In some instances, the surface is also functionalized with a passive material that does not efficiently bind the biomolecule, thereby preventing biomolecule attachment at sites where the passive functionalization agent is bound. In some cases, the surface comprises an active layer only defining distinct loci for biomolecule support.

In some instances, the surface is contacted with a mixture of functionalization groups which are in any different ratio. In some instances, a mixture comprises at least 2, 3, 4, 5 or more different types of functionalization agents. In some cases, the ratio of the at least two types of surface functionalization agents in a mixture is about 1:1, 1:2, 1:5. 1:10, 2:10, 3:10, 4:10, 5:10, 6:10, 7:10, 8:10, 9:10, or any other ratio to achieve a desired surface representation of two groups. In some instances, desired surface tensions, wettabilities, water contact angles, and/or contact angles for other suitable solvents are achieved by providing a substrate surface with a suitable ratio of functionalization agents. In some cases, the agents in a mixture are chosen from suitable reactive and inert moieties, thus diluting the surface density of reactive groups to a desired level for downstream reactions. In some instances, the mixture of functionalization reagents comprises one or more reagents that bind to a biomolecule and one or more reagents that do not bind to a biomolecule. Therefore, modulation of the reagents allows for the control of the amount of biomolecule binding that occurs at a distinct area of functionalization.

In some instances, a method for substrate functionalization comprises deposition of a silane molecule onto a surface of a substrate. The silane molecule may be deposited on a high energy surface of the substrate. In some instances the high surface energy region includes a passive functionalization reagent. Methods described herein provide for a silane group to bind the surface, while the rest of the molecule provides a distance from the surface and a free hydroxyl group at the end to which a biomolecule attaches. In some instances, the silane is an organofunctional alkoxysilane molecule. Non-limiting examples of organofunctional alkoxysilane molecules include dimethylchloro-octodecyl-silane, methyldichloro-octodecyl-silane, trichloro-octodecyl-silane, and trimethyl-octodecyl-silane, triethyl-octodecyl-silane. In some instances, the silane is an amino silane. Examples of amino silanes include, without limitation, 11-acetoxyundecyltriethoxysilane, n-decyltriethoxysilane, (3-aminopropyl)trimethoxysilane, (3-aminopropyl)triethoxysilane, glycidyloxypropyl/trimethoxysilane and N-(3-triethoxysilylpropyl)-4-hydroxybutyramide. In some instances, the silane comprises 11-acetoxyundecyltriethoxysilane, n-decyltriethoxysilane, (3-aminopropyl)trimethoxysilane, (3-aminopropyl)triethoxysilane, glycidyloxypropyl/trimethoxysilane, N-(3-triethoxysilylpropyl)-4-hydroxybutyramide, or any combination thereof. In some instances, an active functionalization agent comprises 11-acetoxyundecyltriethoxysilane. In some instances, an active functionalization agent comprises n-decyltriethoxysilane. In some cases, an active functionalization agent comprises glycidyloxypropyltriethoxysilane (GOPS). In some instances, the silane is a fluorosilane. In some instances, the silane is a hydrocarbon silane. In some cases, the silane is 3-iodo-propyltrimethoxysilane. In some cases, the silane is octylchlorosilane.

In some instances, silanization is performed on a surface through self-assembly with organofunctional alkoxysilane molecules. The organofunctional alkoxysilanes are classified according to their organic functions. Non-limiting examples of siloxane functionalizing reagents include hydroxyalkyl siloxanes (silylate surface, functionalizing with diborane and oxidizing the alcohol by hydrogen peroxide), diol (dihydroxyalkyl) siloxanes (silylate surface, and hydrolyzing to diol), aminoalkyl siloxanes (amines require no intermediate functionalizing step), glycidoxysilanes (3-glycidoxypropyl-dimethyl-ethoxysilane, glycidoxy-trimethoxysilane), mercaptosilanes (3-mercaptopropyl-trimethoxysilane, 3-4 epoxycyclohexyl-ethyltrimethoxy silane or 3-mercaptopropyl-methyl-dimethoxysilane), bicyclohepthenyl-trichlorosilane, butyl-aldehydr-trimethoxysilane, or dimeric secondary aminoalkyl siloxanes. Exemplary hydroxyalkyl siloxanes include allyl trichlorochlorosilane turning into 3-hydroxypropyl, or 7-oct-1-enyl trichlorochlorosilane turning into 8-hydroxyoctyl. The diol (dihydroxyalkyl) siloxanes include glycidyl trimethoxysilane-derived (2,3-dihydroxypropyloxy)propyl (GOPS). The aminoalkyl siloxanes include 3-aminopropyl trimethoxysilane turning into 3-aminopropyl (3-aminopropyl-triethoxysilane, 3-aminopropyl-diethoxy-methylsilane, 3-aminopropyl-dimethyl-ethoxysilane, or 3-aminopropyl-trimethoxysilane). In some cases, the dimeric secondary aminoalkyl siloxanes is bis(3-trimethoxysilylpropyl) amine turning into bis(silyloxylpropyl)amine.

Active functionalization areas may comprise one or more different species of silanes, for example, 1, 2, 3, 4, 5, 6, 7, 8, 9, 10 or more silanes. In some cases, one of the one or more silanes is present in the functionalization composition in an amount greater than another silane. For example, a mixed silane solution having two silanes comprises a 99:1, 98:2, 97:3, 96:4, 95:5, 94:6, 93:7, 92:8, 91:9, 90:10, 89:11, 88:12, 87:13, 86:14, 85:15, 84:16, 83:17, 82:18, 81:19, 80:20, 75:25, 70:30, 65:35, 60:40, 55:45 ratio of one silane to another silane. In some instances, an active functionalization agent comprises 11-acetoxyundecyltriethoxysilane and n-decyltriethoxysilane. In some instances, an active functionalization agent comprises 11-acetoxyundecyltriethoxysilane and n-decyltriethoxysilane in a ratio from about 20:80 to about 1:99, or about 10:90 to about 2:98, or about 5:95.

In some instances, functionalization comprises deposition of a functionalization agent to a structure by any deposition technique, including, but not limiting to, chemical vapor deposition (CVD), atomic layer deposition (ALD), plasma enhanced CVD (PECVD), plasma enhanced ALD (PEALD), metal organic CVD (MOCVD), hot wire CVD (HWCVD), initiated CVD (iCVD), modified CVD (MCVD), vapor axial deposition (VAD), outside vapor deposition (OVD), physical vapor deposition (e.g., sputter deposition, evaporative deposition), and molecular layer deposition (MLD).

Any step or component in the following functionalization process be omitted or changed in accordance with properties desired of the final functionalized substrate. In some cases, additional components and/or process steps are added to the process workflows embodied herein. In some instances, a substrate is first cleaned, for example, using a piranha solution. An example of a cleaning process includes soaking a substrate in a piranha solution (e.g., 90% H₂SO₄, 10% H₂O₂) at an elevated temperature (e.g., 120° C.) and washing (e.g., water) and drying the substrate (e.g., nitrogen gas). The process optionally includes a post piranha treatment comprising soaking the piranha treated substrate in a basic solution (e.g., NH₄OH) followed by an aqueous wash (e.g., water). In some instances, a surface of a structure is plasma cleaned, optionally following the piranha soak and optional post piranha treatment.

An example of a plasma cleaning process comprises an oxygen plasma etch. In some instances, the surface is deposited with an active functionalization agent following by vaporization. In some instances, the substrate is actively functionalized prior to cleaning, for example, by piranha treatment and/or plasma cleaning.

The process for surface functionalization optionally comprises a resist coat and a resist strip. In some instances, following active surface functionalization, the substrate is spin coated with a resist, for example, SPRT™ 3612 positive photoresist. The process for surface functionalization, in various instances, comprises lithography with patterned functionalization. In some instances, photolithography is performed following resist coating. In some instances, after lithography, the surface is visually inspected for lithography defects. The process for surface functionalization, in some instances, comprises a cleaning step, whereby residues of the substrate are removed, for example, by plasma cleaning or etching. In some instances, the plasma cleaning step is performed at some step after the lithography step.

In some instances, a surface coated with a resist is treated to remove the resist, for example, after functionalization and/or after lithography. In some cases, the resist is removed with a solvent, for example, with a stripping solution comprising N-methyl-2-pyrrolidone. In some cases, resist stripping comprises sonication or ultrasonication. In some instances, a resist is coated and stripped, followed by active functionalization of the exposed areas to create a desired differential functionalization pattern.

In some instances, the methods and compositions described herein relate to the application of photoresist for the generation of modified surface properties in selective areas, wherein the application of the photoresist relies on the fluidic properties of the surface defining the spatial distribution of the photoresist. Without being bound by theory, surface tension effects related to the applied fluid may define the flow of the photoresist. For example, surface tension and/or capillary action effects may facilitate drawing of the photoresist into small structures in a controlled fashion before the resist solvents evaporate. In some instances, resist contact points are pinned by sharp edges, thereby controlling the advance of the fluid. The underlying structures may be designed based on the desired flow patterns that are used to apply photoresist during the manufacturing and functionalization processes. A solid organic layer left behind after solvents evaporate may be used to pursue the subsequent steps of the manufacturing process. Structures may be designed to control the flow of fluids by facilitating or inhibiting wicking effects into neighboring fluidic paths. For example, a structure is designed to avoid overlap between top and bottom edges, which facilitates the keeping of the fluid in top structures allowing for a particular disposition of the resist. In an alternative example, the top and bottom edges overlap, leading to the wicking of the applied fluid into bottom structures. Appropriate designs may be selected accordingly, depending on the desired application of the resist.

In some instances, a structure described herein has a surface that comprises a material having thickness of at least or at least 0.1 nm, 0.5 nm, 1 nm, 2 nm, 5 nm, 10 nm or 25 nm that comprises a reactive group capable of binding nucleosides. Exemplary include, without limitation, glass and silicon, such as silicon dioxide and silicon nitride. In some cases, exemplary surfaces include nylon and PMMA.

In some instances, electromagnetic radiation in the form of UV light is used for surface patterning. In some instances, a lamp is used for surface patterning, and a mask mediates exposure locations of the UV light to the surface. In some instances, a laser is used for surface patterning, and a shutter opened/closed state controls exposure of the UV light to the surface. The laser arrangement may be used in combination with a flexible structure that is capable of moving. In such an arrangement, the coordination of laser exposure and flexible structure movement is used to create patterns of one or more agents having differing nucleoside coupling capabilities.

Described herein are surfaces for polynucleotide synthesis that are reusable. After synthesis and/or cleavage of polynucleotides, a surface may be bathed, washed, cleaned, baked, etched, or otherwise functionally restored to a condition suitable for subsequent polynucleotide synthesis. The number of times a surface is reused and the methods for recycling/preparing the surface for reuse vary depending on subsequent applications. Surfaces prepared for reuse are in some instances reused at least 1, 2, 3, 5, 10, 20, 50, 100, 1,000 or more times. In some instances, the remaining “life” or number of times a surface is suitable for reuse is measured or predicted.

### Material Deposition Systems

A material deposition system may be used to deposit building blocks, reagents, or both, on a solid support. The material deposition system may comprise a substrate for constructing a DNA sequence; and a deposition unit depositing one or more building blocks, reagents, or both for constructing the DNA sequence. In some instances, the material deposition system is in communication with a computing system. In some examples, the computing system directs the deposition of one or more building blocks or reagents during synthesis or storage of polynucleotides by the material deposition system. In some examples, the material deposition receives input in the form of one or more sequences of polynucleotides from the computing system, and the material deposition system deposits building blocks, reagents, or both to select locations of the solid support. In some examples, the building blocks are nucleotide monomers. In some examples, the building blocks are nucleic acid sequences. The nucleic acid sequences may be about 2, 3, 4, 5, 6, 7, 8, 9, 10, 12, 15, 20, 25, or 30 bases in length. In some examples, the computing system, the material deposition system, or both are in communication with a controller that provides fluidic and/or electronic control of the material deposition system. The material deposition system, computing system, and/or controller may be part of a larger storage system as described herein.

In some cases, the synthesized polynucleotides are stored on the substrate, for example a solid support. Nucleic acid reagents may be deposited on the substrate surface in a non-continuous, or drop-on-demand method using the material deposition system. Examples of such methods include the electromechanical transfer method, electric thermal transfer method, and electrostatic attraction method. In the electromechanical transfer method, piezoelectric elements deformed by electrical pulses cause the droplets to be ejected. In the electric thermal transfer method, bubbles are generated in a chamber of the device, and the expansive force of the bubbles causes the droplets to be ejected. In the electrostatic attraction method, electrostatic force of attraction is used to eject the droplets onto the substrate. In some cases, the drop frequency is from about 5 KHz to about 500 KHz; from about 5 KHz to about 100 KHz; from about 10 KHz to about 500 KHz; from about 10 KHz to about 100 KHz; or from about 50 KHz to about 500 KHz. In some cases, the frequency is less than about 500 KHz, 200 KHz, 100 KHz, or 50 KHz.

The size of the droplets dispensed correlates to the resolution of the device. In some instances, the devices deposit droplets of reagents at sizes from about 0.01 pl to about 20 pl, from about 0.01 pl to about 10 pl, from about 0.01 pl to about 1 pl, from about 0.01 pl to about 0.5 pl, from about 0.01 pl to about 0.01 pl, or from about 0.05 pl to about 1 pl. In some instances, the droplet size is less than about 1 pl, 0.5 pl, 0.2 pl, 0.1 pl, or 0.05 pl.

In some arrangements, the configuration of a polynucleotide synthesis system allows for a continuous polynucleotide synthesis process that exploits the flexibility of a substrate for traveling in a reel-to-reel type process. This synthesis process operates in a continuous production line manner with the substrate travelling through various stages of polynucleotide synthesis using one or more reels to rotate the position of the substrate. In an exemplary instance, a polynucleotide synthesis reaction comprises rolling a substrate: through a solvent bath, beneath a deposition device for phosphoramidite deposition, through a bath of oxidizing agent, through an acetonitrile wash bath, and through a deblock bath. Optionally, the tape is also traversed through a capping bath. A reel-to-reel type process allows for the finished product of a substrate comprising synthesized polynucleotides to be easily gathered on a take-up reel, where it can be transported for further processing or storage.

In some arrangements, polynucleotide synthesis proceeds in a continuous process as a continuous flexible tape is conveyed along a conveyor belt system. Similar to the reel-to-reel type process, polynucleotide synthesis on a continuous tape operates in a production line manner, with the substrate travelling through various stages of polynucleotide synthesis during conveyance. However, in a conveyor belt process, the continuous tape revisits a polynucleotide synthesis step without rolling and unrolling of the tape, as in a reel-to-reel process. In some arrangements, polynucleotide synthesis steps are partitioned into zones and a continuous tape is conveyed through each zone one or more times in a cycle. For example, a polynucleotide synthesis reaction may comprise (1) conveying a substrate through a solvent bath, beneath a deposition device for phosphoramidite deposition, through a bath of oxidizing agent, through an acetonitrile wash bath, and through a block bath in a cycle; and then (2) repeating the cycles to achieve synthesized polynucleotides of a predetermined length. After polynucleotide synthesis, the flexible substrate is removed from the conveyor belt system and, optionally, rolled for storage. Rolling may be around a reel, for storage. In some instances, a flexible substrate comprising thermoplastic material is coated with nucleoside coupling reagent. The coating is patterned into loci such that each locus has diameter of about 10 um, with a center-to-center distance between two adjacent loci of about 21 um. In this instance, the locus size is sufficient to accommodate a sessile drop volume of 0.2 pl during a polynucleotide synthesis deposition step. In some cases, the locus density is about 2.2 billion loci per m² (1 locus/441×10⁻¹² m²). In some cases, a 4.5 m² substrate comprise about 10 billion loci, each with a 10 um diameter.

In some arrangements, a device (e.g., deposition unit) for application of one or more reagents to a substrate during a synthesis reaction is configured to deposit reagents and/or nucleoside monomers for nucleoside phosphoramidite based synthesis. Reagents for polynucleotide synthesis include reagents for polynucleotide extension and wash buffers. As non-limiting examples, the device deposits cleaning reagents, coupling reagents, capping reagents, oxidizers, de-blocking agents, acetonitrile, gases such as nitrogen gas, and any combination thereof. In addition, the device optionally deposits reagents for the preparation and/or maintenance of substrate integrity. In some instances, the polynucleotide synthesizer deposits a drop having a diameter less than about 200 um, 100 um, or 50 um in a volume less than about 1000, 500, 100, 50, or 20 pl. In some cases, the polynucleotide synthesizer deposits between about 1 and 10000, 1 and 5000, 100 and 5000, or 1000 and 5000 droplets per second.

Described herein are devices, methods, systems and compositions where reagents for polynucleotide synthesis are recycled or reused. Recycling of reagents may comprise collection, storage, and usage of unused reagents, or purification/transformation of used reagents. For example, a reagent bath is recycled and used for a polynucleotide synthesis step on the same or a different surface. Reagents described herein may be recycled 1, 2, 3, 4, 5, 6, 7, 8, 9, 10 or more times. Alternatively or in combination, a reagent solution comprising a reaction byproduct is filtered to remove the byproduct, and the reagent solution is used for additional polynucleotide synthesis reactions.

Many integrated or non-integrated elements are often used with polynucleotide synthesis systems. In some instances, a polynucleotide synthesis system comprises one or more elements useful for downstream processing of synthesized polynucleotides. As an example, the system comprises a temperature control element such as a thermal cycling device. In some instances, the temperature control element is used with a plurality of resolved reactors to perform nucleic acid assembly such as PCA and/or nucleic acid amplification such as PCR.

### De Novo Polynucleotide Synthesis

Provided herein are systems and methods for synthesis of a high density of polynucleotides on a substrate in a short amount of time. In some instances, the substrate is a flexible substrate. In some instances, at least 10¹⁰, 10¹¹, 10¹², 10¹³, 10¹⁴, or 10¹⁵ bases are synthesized in one day. In some instances, at least 10×10⁸, 10×10⁹, 10×10¹⁰, 10×10¹¹, or 10×10¹² polynucleotides are synthesized in one day. In some cases, each polynucleotide synthesized comprises at least 20, 25, 50, 100, 200, 300, 400 or at least 500 nucleobases. In some cases, each polynucleotide synthesized comprises 20-500, 25-500, 50-500, 100-500, 200-500, 300-500, 400-500 50-250, 50-300, 100-300, or 150-400 bases. In some cases, these bases are synthesized with a total average error rate of less than about 1 in 100; 200; 300; 400; 500; 1000; 2000; 5000; 10000; 15000; 20000 bases. In some instances, these error rates are for at least 50%, 60%, 70%, 80%, 90%, 95%, 98%, 99%, 99.5%, or more of the polynucleotides synthesized. In some instances, these at least 90%, 95%, 98%, 99%, 99.5%, or more of the polynucleotides synthesized do not differ from a predetermined sequence for which they encode. In some instances, the error rate for synthesized polynucleotides on a substrate using the methods and systems described herein is less than about 1 in 200. In some instances, the error rate for synthesized polynucleotides on a substrate using the methods and systems described herein is less than about 1 in 1,000. In some instances, the error rate for synthesized polynucleotides on a substrate using the methods and systems described herein is less than about 1 in 2,000. In some instances, the error rate for synthesized polynucleotides on a substrate using the methods and systems described herein is less than about 1 in 3,000. In some instances, the error rate for synthesized polynucleotides on a substrate using the methods and systems described herein is less than about 1 in 5,000. Individual types of error rates include mismatches, deletions, insertions, and/or substitutions for the polynucleotides synthesized on the substrate. The term “error rate” refers to a comparison of the collective amount of synthesized polynucleotide to an aggregate of predetermined polynucleotide sequences. In some instances, synthesized polynucleotides disclosed herein comprise a tether of 12 to 25 bases. In some instances, the tether comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more bases.

Described herein are methods, systems, devices, and compositions wherein chemical reactions used in polynucleotide synthesis are controlled using electrochemistry. Electrochemical reactions in some instances are controlled by any source of energy, such as light, heat, radiation, or electricity. For example, electrodes are used to control chemical reactions as all or a portion of discrete loci on a surface. Electrodes in some instances are charged by applying an electrical potential to the electrode to control one or more chemical steps in polynucleotide synthesis. In some instances, these electrodes are addressable. Any number of the chemical steps described herein is in some instances controlled with one or more electrodes. Electrochemical reactions may comprise oxidations, reductions, acid/base chemistry, or other reaction that is controlled by an electrode. In some instances, electrodes generate electrons or protons that are used as reagents for chemical transformations. Electrodes in some instances directly generate a reagent such as an acid. In some instances, an acid is a proton. Electrodes in some instances directly generate a reagent such as a base. Acids or bases are often used to cleave protecting groups, or influence the kinetics of various polynucleotide synthesis reactions, for example by adjusting the pH of a reaction solution. Electrochemically controlled polynucleotide synthesis reactions in some instances comprise redox-active metals or other redox-active organic materials. In some instances, metal or organic catalysts are employed with these electrochemical reactions. In some instances, acids are generated from oxidation of quinones.

Control of chemical reactions with is not limited to the electrochemical generation of reagents; chemical reactivity may be influenced indirectly through biophysical changes to substrates or reagents through electric fields (or gradients) which are generated by electrodes. In some instances, substrates include but are not limited to nucleic acids. In some instances, electrical fields which repel or attract specific reagents or substrates towards or away from an electrode or surface are generated. Such fields in some instances are generated by application of an electrical potential to one or more electrodes. For example, negatively charged nucleic acids are repelled from negatively charged electrode surfaces. Such repulsions or attractions of polynucleotides or other reagents caused by local electric fields in some instances provides for movement of polynucleotides or other reagents in or out of region of the synthesis device or structure. In some instances, electrodes generate electric fields which repel polynucleotides away from a synthesis surface, structure, or device. In some instances, electrodes generate electric fields which attract polynucleotides towards a synthesis surface, structure, or device. In some instances, protons are repelled from a positively charged surface to limit contact of protons with substrates or portions thereof. In some instances, repulsion or attractive forces are used to allow or block entry of reagents or substrates to specific areas of the synthesis surface. In some instances, nucleoside monomers are prevented from contacting a polynucleotide chain by application of an electric field in the vicinity of one or both components. Such arrangements allow gating of specific reagents, which may obviate the need for protecting groups when the concentration or rate of contact between reagents and/or substrates is controlled. In some instances, unprotected nucleoside monomers are used for polynucleotide synthesis. Alternatively, application of the field in the vicinity of one or both components promotes contact of nucleoside monomers with a polynucleotide chain. Additionally, application of electric fields to a substrate can alter the substrates reactivity or conformation. In an exemplary application, electric fields generated by electrodes are used to prevent polynucleotides at adjacent loci from interacting. In some instances, the substrate is a polynucleotide, optionally attached to a surface. Application of an electric field in some instances alters the three-dimensional structure of a polynucleotide. Such alterations comprise folding or unfolding of various structures, such as helices, hairpins, loops, or other 3-dimensional nucleic acid structure. Such alterations are useful for manipulating nucleic acids inside of wells, channels, or other structures. In some instances, electric fields are applied to a nucleic acid substrate to prevent secondary structures. In some instances, electric fields obviate the need for linkers or attachment to a solid support during polynucleotide synthesis.

A suitable method for polynucleotide synthesis on a substrate of this disclosure is a phosphoramidite method comprising the controlled addition of a phosphoramidite building block, i.e. nucleoside phosphoramidite, to a growing polynucleotide chain in a coupling step that forms a phosphite triester linkage between the phosphoramidite building block and a nucleoside bound to the substrate. In some instances, the nucleoside phosphoramidite is provided to the substrate activated. In some instances, the nucleoside phosphoramidite is provided to the substrate with an activator. In some instances, nucleoside phosphoramidites are provided to the substrate in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition and linkage of a nucleoside phosphoramidite in the coupling step, the substrate is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the substrate is de-protected by removal of a protecting group, where the protecting group functions to prevent polymerization. Protecting groups may comprise any chemical group that prevents extension of the polynucleotide chain. In some instances, the protecting group is cleaved (or removed) in the presence of an acid. In some instances, the protecting group is cleaved in the presence of a base. In some instances, the protecting group is removed with electromagnetic radiation such as light, heat, or other energy source. In some instances, the protecting group is removed through an oxidation or reduction reaction. In some instances, a protecting group comprises a triarylmethyl group. In some instances, a protecting group comprises an aryl ether. In some instances, a protecting comprises a disulfide. In some instances a protecting group comprises an acid-labile silane. In some instances, a protecting group comprises an acetal. In some instances, a protecting group comprises a ketal. In some instances, a protecting group comprises an enol ether. In some instances, a protecting group comprises a methoxybenzyl group. In some instances, a protecting group comprises an azide. In some instances, a protecting group is 4,4′-dimethoxytrityl (DMT). In some instances, a protecting group is a tert-butyl carbonate. In some instances, a protecting group is a tert-butyl ester. In some instances, a protecting group comprises a base-labile group.

Following coupling, phosphoramidite polynucleotide synthesis methods optionally comprise a capping step. In a capping step, the growing polynucleotide is treated with a capping agent. A capping step generally serves to block unreacted substrate-bound 5′-OH groups after coupling from further chain elongation, preventing the formation of polynucleotides with internal base deletions. Further, phosphoramidites activated with 1H-tetrazole often react, to a small extent, with the O6 position of guanosine. Without being bound by theory, upon oxidation with I₂/water, this side product, possibly via O6-N7 migration, undergoes depurination. The apurinic sites can end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the substrate is optionally washed.

Following addition of a nucleoside phosphoramidite, and optionally after capping and one or more wash steps, a substrate described herein comprises a bound growing nucleic acid that may be oxidized. The oxidation step comprises oxidizing the phosphite triester into a tetracoordinated phosphate triester, a protected precursor of the naturally occurring phosphate diester internucleoside linkage. In some instances, phosphite triesters are oxidized electrochemically. In some instances, oxidation of the growing polynucleotide is achieved by treatment with iodine and water, optionally in the presence of a weak base such as a pyridine, lutidine, or collidine. Oxidation is sometimes carried out under anhydrous conditions using tert-Butyl hydroperoxide or (1S)-(+)-(10-camphorsulfonyl)-oxaziridine (CSO). In some methods, a capping step is performed following oxidation. A second capping step allows for substrate drying, as residual water from oxidation that may persist can inhibit subsequent coupling. Following oxidation, the substrate and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including, but not limited to, 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

For a subsequent cycle of nucleoside incorporation to occur through coupling, a protected 5′ end (or 3′ end, if synthesis is conducted in a 5′ to 3′ direction) of the substrate bound growing polynucleotide is be removed so that the primary hydroxyl group can react with a next nucleoside phosphoramidite. In some instances, the protecting group is DMT and deblocking occurs with trichloroacetic acid in dichloromethane. In some instances, the protecting group is DMT and deblocking occurs with electrochemically generated protons. Conducting detritylation for an extended time or with stronger than recommended solutions of acids may lead to increased depurination of solid support-bound polynucleotide and thus reduces the yield of the desired full-length product. Methods and compositions described herein provide for controlled deblocking conditions limiting undesired depurination reactions. In some instances, the substrate bound polynucleotide is washed after deblocking. In some cases, efficient washing after deblocking contributes to synthesized polynucleotides having a low error rate.

Methods for the synthesis of polynucleotides on a substrate described herein may involve an iterating sequence of the following steps: application of a protected monomer to a surface of a substrate feature to link with either the surface, a linker or with a previously deprotected monomer; deprotection of the applied monomer so that it can react with a subsequently applied protected monomer; and application of another protected monomer for linking. One or more intermediate steps include oxidation and/or sulfurization. In some instances, one or more wash steps precede or follow one or all of the steps.

Methods for the synthesis of polynucleotides on a substrate described herein may comprise an oxidation step. For example, methods involve an iterating sequence of the following steps: application of a protected monomer to a surface of a substrate feature to link with either the surface, a linker or with a previously deprotected monomer; deprotection of the applied monomer so that it can react with a subsequently applied protected monomer; application of another protected monomer for linking, and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer; deprotection of the applied monomer so that it can react with a subsequently applied protected monomer; and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer; and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer; deprotection of the applied monomer so that it can react with a subsequently applied protected monomer; and oxidation and/or sulfurization. In some instances, one or more wash steps precede or follow one or all of the steps.

In some instances, polynucleotides are synthesized with photolabile protecting groups, where the hydroxyl groups generated on the surface are blocked by photolabile-protecting groups. When the surface is exposed to UV light, such as through a photolithographic mask, a pattern of free hydroxyl groups on the surface may be generated. These hydroxyl groups can react with photoprotected nucleoside phosphoramidites, according to phosphoramidite chemistry. A second photolithographic mask can be applied and the surface can be exposed to UV light to generate second pattern of hydroxyl groups, followed by coupling with 5′-photoprotected nucleoside phosphoramidite. Likewise, patterns can be generated and oligomer chains can be extended. Without being bound by theory, the lability of a photocleavable group depends on the wavelength and polarity of a solvent employed and the rate of photocleavage may be affected by the duration of exposure and the intensity of light. This method can leverage a number of factors such as accuracy in alignment of the masks, efficiency of removal of photo-protecting groups, and the yields of the phosphoramidite coupling step. Further, unintended leakage of light into neighboring sites can be minimized. The density of synthesized oligomer per spot can be monitored by adjusting loading of the leader nucleoside on the surface of synthesis.

The surface of a substrate described herein that provides support for polynucleotide synthesis may be chemically modified to allow for the synthesized polynucleotide chain to be cleaved from the surface. In some instances, the polynucleotide chain is cleaved at the same time as the polynucleotide is deprotected. In some cases, the polynucleotide chain is cleaved after the polynucleotide is deprotected. In an exemplary scheme, a trialkoxysilyl amine such as (CH₃CH₂O)₃Si—(CH₂)₂—NH₂ is reacted with surface SiOH groups of a substrate, followed by reaction with succinic anhydride with the amine to create an amide linkage and a free OH on which the nucleic acid chain growth is supported. Cleavage includes gas cleavage with ammonia or methylamine. In some instances cleavage includes linker cleavage with electrically generated reagents such as acids or bases. In some instances, once released from the surface, polynucleotides are assembled into larger nucleic acids that are sequenced and decoded to extract stored information.

The surfaces described herein can be reused after polynucleotide cleavage to support additional cycles of polynucleotide synthesis. For example, the linker can be reused without additional treatment/chemical modifications. In some instances, a linker is non-covalently bound to a substrate surface or a polynucleotide. In some embodiments, the linker remains attached to the polynucleotide after cleavage from the surface. Linkers in some embodiments comprise reversible covalent bonds such as esters, amides, ketals, beta substituted ketones, heterocycles, or other group that is capable of being reversibly cleaved. Such reversible cleavage reactions are in some instances controlled through the addition or removal of reagents, or by electrochemical processes controlled by electrodes. Optionally, chemical linkers or surface-bound chemical groups are regenerated after a number of cycles, to restore reactivity and remove unwanted side product formation on such linkers or surface-bound chemical groups.

### Assembly

Polynucleotides may be designed to collectively span a large region of a predetermined sequence that encodes for information. In some instances, larger polynucleotides are generated through ligation reactions to join the synthesized polynucleotides. Ligation reactions may be performed using a material deposition system, as provided herein. One example of a ligation reaction is polymerase chain assembly (PCA). In some instances, at least of a portion of the polynucleotides are designed to include an appended region that is a substrate for universal primer binding. For PCA reactions, the presynthesized polynucleotides include overlaps with each other (e.g., 4, 20, 40 or more bases with overlapping sequence). During the polymerase cycles, the polynucleotides anneal to complementary fragments and then are filled in by polymerase. Each cycle thus increases the length of various fragments randomly depending on which polynucleotides find each other. Complementarity amongst the fragments allows for forming a complete large span of double-stranded DNA. In some cases, after the PCA reaction is complete, an error correction step is conducted using mismatch repair detecting enzymes to remove mismatches in the sequence. Once larger fragments of a target sequence are generated, they can be amplified. For example, in some cases, a target sequence comprising 5′ and 3′ terminal adapter sequences is amplified in a polymerase chain reaction (PCR) which includes modified primers that hybridize to the adapter sequences. In some cases, the modified primers comprise one or more uracil bases. The use of modified primers allows for removal of the primers through enzymatic reactions centered on targeting the modified base and/or gaps left by enzymes which cleave the modified base pair from the fragment. What remains is a double-stranded amplification product that lacks remnants of adapter sequence. In this way, multiple amplification products can be generated in parallel with the same set of primers to generate different fragments of double-stranded DNA.

Error correction may be performed on synthesized polynucleotides and/or assembled products. An example strategy for error correction involves site-directed mutagenesis by overlap extension PCR to correct errors, which is optionally coupled with two or more rounds of cloning and sequencing. In certain instances, double-stranded nucleic acids with mismatches, bulges and small loops, chemically altered bases and/or other heteroduplexes are selectively removed from populations of correctly synthesized nucleic acids. In some instances, error correction is performed using proteins/enzymes that recognize and bind to or next to mismatched or unpaired bases within double-stranded nucleic acids to create a single or double-strand break or to initiate a strand transfer transposition event. Non-limiting examples of proteins/enzymes for error correction include endonucleases (T7 Endonuclease I, E. coli Endonuclease V, T4 Endonuclease VII, mung bean nuclease, Cell, E. coli Endonuclease IV, UVDE), restriction enzymes, glycosylases, ribonucleases, mismatch repair enzymes, resolvases, helicases, ligases, antibodies specific for mismatches, and their variants. Examples of specific error correction enzymes include T4 endonuclease 7, T7 endonuclease 1, S1, mung bean endonuclease, MutY, MutS, MutH, MutL, cleavase, CELI, and HINF1. In some cases, DNA mismatch-binding protein MutS (Thermus aquaticus) is used to remove failure products from a population of synthesized products. In some instances, error correction is performed using the enzyme Correctase. In some cases, error correction is performed using SURVEYOR endonuclease (Transgenomic), a mismatch-specific DNA endonuclease that scans for known and unknown mutations and polymorphisms for heteroduplex DNA.

### Sequencing

After extraction and/or amplification of polynucleotides from the surface of the structure, suitable sequencing technology may be employed to sequence the polynucleotides. In some cases, the DNA sequence is read on the substrate or within a feature of a structure. In some cases, the polynucleotides stored on the substrate are extracted is optionally assembled into longer nucleic acids and then sequenced.

Polynucleotides synthesized and stored on the structures described herein encode data that can be interpreted by reading the sequence of the synthesized polynucleotides and converting the sequence into binary code readable by a computer. In some cases the sequences require assembly, and the assembly step may need to be at the nucleic acid sequence stage or at the digital sequence stage.

Provided herein are detection systems comprising a device capable of sequencing stored polynucleotides, either directly on the structure and/or after removal from the main structure. In cases where the structure is a reel-to-reel tape of flexible material, the detection system comprises a device for holding and advancing the structure through a detection location and a detector disposed proximate the detection location for detecting a signal originated from a section of the tape when the section is at the detection location. In some instances, the signal is indicative of a presence of a polynucleotide. In some instances, the signal is indicative of a sequence of a polynucleotide (e.g., a fluorescent signal). In some instances, information encoded within polynucleotides on a continuous tape is read by a computer as the tape is conveyed continuously through a detector operably connected to the computer. In some instances, a detection system comprises a computer system comprising a polynucleotide sequencing device, a database for storage and retrieval of data relating to polynucleotide sequence, software for converting DNA code of a polynucleotide sequence to binary code, a computer for reading the binary code, or any combination thereof.

Provided herein are sequencing systems that can be integrated into the devices described herein. Various methods of sequencing are well known in the art, and comprise “base calling” wherein the identity of a base in the targetpolynucleotide is identified. In some instances, polynucleotides synthesized using the methods, devices, compositions, and systems described herein are sequenced after cleavage from the synthesis surface. In some instances, sequencing occurs during or simultaneously with polynucleotide synthesis, wherein base calling occurs immediately after or before extension of a nucleoside monomer into the growing polynucleotide chain. Methods for base calling include measurement of electrical currents/voltages generated by polymerase-catalyzed addition of bases to a template strand. In some instances, synthesis surfaces comprise enzymes, such as polymerases. In some instances, such enzymes are tethered to electrodes or to the synthesis surface.

### Storage System

The polynucleotides constructed using the system, devices, and methods described herein may be stored in a data storage system. A system for data storage can comprise one or more modules. In some instances, the some or all of the one or more modules are in communication. In some examples, some or all of the one or more modules are in communication to allow transferring of polynucleotides between them. In some examples, some or all of the one or more modules are fluidically coupled. In some examples, some or all of the one or more modules are fluidically coupled with one or more tubes. A fluid may generally refer to one or more liquids used in various processes involved in handling polynucleotides, including, without limitation, synthesis, amplification, preparation for sequencing, and sequencing. In some examples, some or all of the modules are in communication to allow transferring of control commands between modules of the system. In some examples, some or all of the one or more modules are electronically coupled. A module in the system can comprise, without limitation, a material deposition system, a synthesizer unit, an amplification chamber, a sequencer unit, a storage unit, a controller, a robotic system, a computing system, or any combination thereof. In some examples, a module can further comprise a fluid source, a database or a file system, or both. In some examples, the database or file system keeps track of the storage capacity of the system. For example, the database or file system can keep track of available racks (or trays), slots (for capsules), or both. In some examples, the database or the file system is used to determine the disposition of the rack within the storage system. In some instances, movement of polynucleotides between one or more modules of a system is accomplished by one or more tubes or a robotic system. In some examples, the database or the file system is used to direct the robotic system to the correct position in the storage system. In some instances, the system is autonomous.

In some instances, solid supports or compartments comprising polynucleotides are interfaced together on a larger unit. Interfacing may comprise exchange of fluids, electrical signals, or other medium of exchange between solid supports. This unit is capable of interface with any number of servers, computers, or networked devices. For example, a plurality of solid support is integrated onto a rack unit, which is conveniently inserted or removed from a server rack. The rack unit may comprise any number of solid supports. In some instances the rack unit comprises at least 1, 2, 5, 10, 20, 50, 100, 200, 500, 1000, 2000, 5000, 10,000, 20,000, 50,000, 100,000 or more than 100,000 solid supports or compartments comprising polynucleotides. In some instances, two or more solid supports or compartments comprising polynucleotides are not interfaced with each other. Polynucleotides (and the information stored in them) present on solid supports can be accessed from the rack unit. Access includes removal of polynucleotides from solid supports or compartments, direct analysis of polynucleotides on the solid support or compartment, or any other method which allows the information stored in the polynucleotides to be manipulated or identified. Information in some instances is accessed from a plurality of racks, a single rack, a single solid support or compartment, a portion of the solid support or compartment, or a single locus on a solid support. In various instances, access comprises interfacing polynucleotides with additional devices such as mass spectrometers, HPLC, sequencing instruments, PCR thermocyclers, or other device for manipulating polynucleotides. Access to nucleic acid information in some instances is achieved by cleavage of polynucleotides from all or a portion of a solid support or compartment. Cleavage in some instances comprises exposure to chemical reagents (ammonia or other reagent), electrical potential, radiation, heat, light, acoustics, or other form of energy capable of manipulating chemical bonds. In some instances, cleavage occurs by charging one or more electrodes in the vicinity of the polynucleotides. In some instances, electromagnetic radiation in the form of UV light is used for cleavage of polynucleotides. In some instances, a lamp is used for cleavage of polynucleotides, and a mask mediates exposure locations of the UV light to the surface. In some instances, a laser is used for cleavage of polynucleotides, and a shutter opened/closed state controls exposure of the UV light to the surface. In some instances, access to nucleic acid information (including removal/addition of racks, solid supports, reagents, polynucleotides, or other component) is completely automated.

A structure comprising a plurality of polynucleotides can be stored in an identifiable layout in storage unit. The identifiable layout may comprise a rack or a plurality of racks, or a variation thereof The rack may be used to hold one or more structures comprising the plurality of polynucleotides. In some instances, each structure is stored at a fixed location in the identifiable layout. In some instances, the tag comprises information about a location of the structure in the identifiable layout. As an example, a tag (e.g., RFID) or barcode can encode metadata comprising a location of the structure in the identifiable layout. In some instances, the rack may be located in a data center. In some instances, the rack uses mechanical structures commonly used for mounting conventional computing and data storage resources in rack units. For example, a rack may comprise openings adapted to support disk drives, processing blades, and/or other computer equipment. In some instances, the storage unit may be accessed using a robotic system and/or a controller. In some instances, the identifiable layout in the storage unit comprises robotically addressable slots. Each slot may hold a structure comprising a plurality of polynucleotides. In some instances, each slot comprises a width, depth, length, or any combination thereof for accommodating a structure comprising the plurality of polynucleotides. In some instances, a rack comprises a plurality of slots, where each slot holds a structure comprising the plurality of polynucleotides. In some instances, the controller is capable of cataloguing all storage structures loaded, unloaded, and/or stored within a rack or the slots.

### Computer Systems

In various aspects, any of the systems described herein are operably linked to a computer and are optionally automated through a computer either locally or remotely. In various instances, the methods and systems described herein further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure provided herein. In some instances, the computer systems are programmed to interface between the user specified base sequence and the position of a material deposition device to deliver the correct building blocks and/or reagents to specified regions of the substrate (e.g., specific loci).

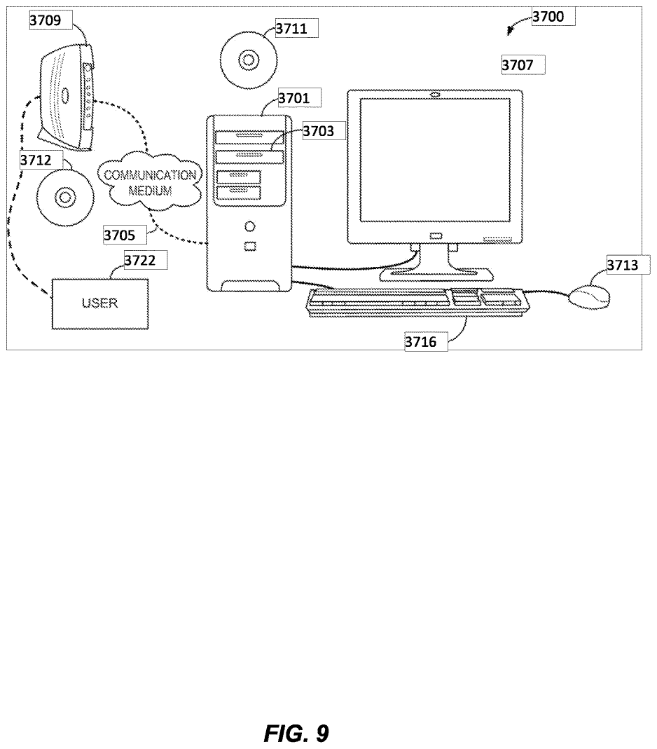

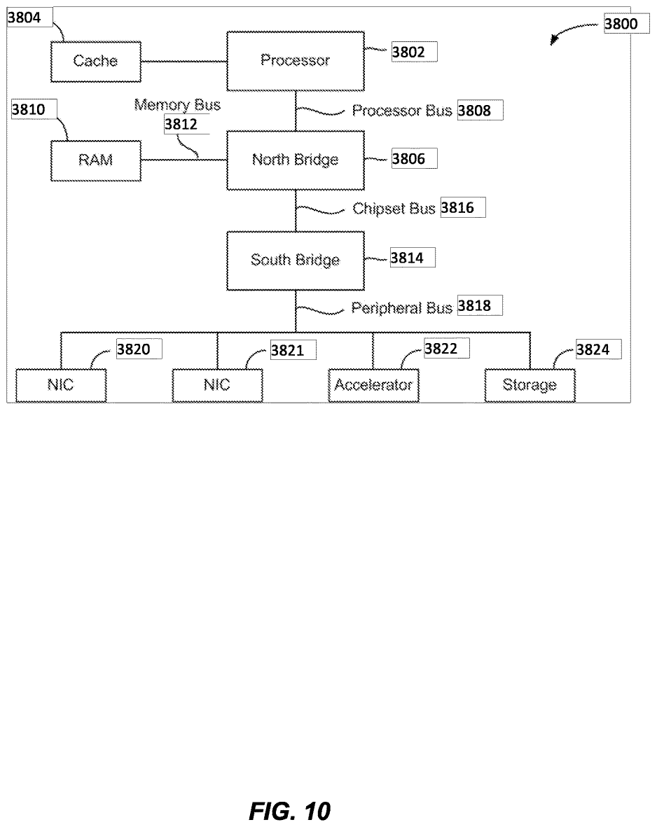

As an example, a computer system, such as the system shown in FIG. 9 or FIG. 10, may be used for encoding data represented as a set of symbols to another set of symbols. For example, the data may be represented as numerical symbols, such as binary values of “0”s and “1”s and the computer system may execute a program comprising a codec (e.g., an error correction code, such as RS code, LDPC code, Turbo code, etc.). In some instances, the computer system converts a first string of symbols to a second string of symbols using the program. In some instances, the computer system executes a program to convert the data to a plurality of nucleic acid sequences, convert a plurality of nucleic acid sequences to data, or both. In some instances, the computer system executes a program to convert a first one or more nucleic acid sequence to a second one or more nucleic acid sequences. For example, the computer program may convert a first one or more nucleic acid sequence to a second one or more nucleic acid sequence, where the second one or more nucleic acid sequences is more resistant to oxidation compared to the first one or more nucleic acid sequences.

A program may be a machine learning algorithm. In some examples, the machine learning algorithm is used to estimate one or more properties of a nucleic acid sequence during synthesis or storage. Non-limiting examples of properties of nucleic acid sequences include stability, resiliency, fidelity, degradation, or error rates, to various temperatures, redox conditions, humidity, or any other synthesis or storage condition for a given period of time. As an example, a machine learning algorithm may determine which one or more guanosine bases to substitute for inosine bases in a nucleic acid sequence in order to increase the redox resistance and decrease error rates during synthesis or storage of the polynucleotides comprising the nucleic acid sequence. In some examples, the machine learning algorithm can be used to determine a nucleotide base based on a signal (e.g., electrical signal, such as current or voltage).

A program may be executed on a computer system provided herein. In some instances, a program comprises a statistical algorithm or a machine learning algorithm. In some instances, an algorithm comprising machine learning (ML) is used to associate the signal (e.g., electrical currents/voltages) to the nucleoside monomer added to the polynucleotide. In some cases, the algorithm comprising ML may be trained with training data in order to associate the signal (e.g., electrical currents/voltages) to the nucleoside monomer added to the polynucleotide. In some cases, the algorithm comprises classical ML algorithms for classification and/or clustering (e.g., K-means clustering, mean-shift clustering, density-based spatial clustering of applications with noise (DBSCAN), expectation-maximization (EM) clustering, agglomerative hierarchical clustering, logistic regression, naïve Bayes, K-nearest neighbors, random forests or decision trees, gradient boosting, support vector machines (SVMs), or a combination thereof).

In some cases, the algorithm comprises a learning algorithm comprising layers, such as one or more neural networks. Neural networks may comprise connected nodes in a network, which may perform functions, such as transforming or translating input data. In some examples, the output from a given node may be passed on as input to another node. In some embodiments, the nodes in the network may comprise input units, hidden units, output units, or a combination thereof. In some cases, an input node may be connected to one or more hidden units. In some cases, one or more hidden units may be connected to an output unit. The nodes may take in input and may generate an output based on an activation function. In some embodiments, the input or output may be a tensor, a matrix, a vector, an array, or a scalar. In some embodiments, the activation function may be a Rectified Linear Unit (ReLU) activation function, a sigmoid activation function, or a hyperbolic tangent activation function. In some embodiments, the activation function may be a Softmax activation function. The connections between nodes may further comprise weights for adjusting input data to a given node (e.g., to activate input data or deactivate input data). In some embodiments, the weights may be learned by the neural network. In some embodiments, the neural network may be trained using gradient-based optimizations. In some cases, the gradient-based optimization may comprise of one or more loss functions. In some examples, the gradient-based optimization may be conjugate gradient descent, stochastic gradient descent, or a variation thereof (e.g., adaptive moment estimation (Adam)). In further examples, the gradient in the gradient-based optimization may be computed using backpropagation. In some embodiments, the nodes may be organized into graphs to generate a network (e.g., graph neural networks). In some embodiments, the nodes may be organized into one or more layers to generate a network (e.g., feed forward neural networks, convolutional neural networks (CNNs), recurrent neural networks (RNNs), etc.). In some cases, the neural network may be a deep neural network comprising of more than one layer.

In some cases, the neural network may comprise one or more recurrent layer. In some examples, the one or more recurrent layer may be one or more long short-term memory (LSTM) layers or gated recurrent unit (GRU), which may perform sequential data classification and clustering. In some embodiments, the neural network may comprise one or more convolutional layers. The input and output may be a tensor representing of variables or attributes in a data set (e.g., features), which may be referred to as a feature map (or activation map). In some cases, the convolutions may be one dimensional (1D) convolutions, two dimensional (2D) convolutions, three dimensional (3D) convolutions, or any combination thereof. In further cases, the convolutions may be 1D transpose convolutions, 2D transpose convolutions, 3D transpose convolutions, or any combination thereof. In some examples, one-dimensional convolutional layers may be suited for time series data since it may classify time series through parallel convolutions. In some examples, convolutional layers may be used for analyzing a signal (e.g., electrical currents/voltages) to the nucleoside monomer added to the polynucleotide.

The layers in a neural network may further comprise one or more pooling layers before or after a convolutional layer. The one or more pooling layers may reduce the dimensionality of the feature map using filters that summarize regions of a matrix. This may down sample the number of outputs, and thus reduce the parameters and computational resources needed for the neural network. In some embodiments, the one or more pooling layers may be max pooling, min pooling, average pooling, global pooling, norm pooling, or a combination thereof. Max pooling may reduce the dimensionality of the data by taking only the maximums values in the region of the matrix, which helps capture the significant feature. In some embodiments, the one or more pooling layers may be one dimensional (1D), two dimensional (2D), three dimensional (3D), or any combination thereof. The neural network may further comprise of one or more flattening layers, which may flatten the input to be passed on to the next layer. In some cases, the input may be flattened by reducing it to a one-dimensional array. The flattened inputs may be used to output a classification of an object (e.g., classification of signals (e.g., electrical currents/voltages) to a nucleoside monomer added to the polynucleotide, etc.). The neural networks may further comprise one or more dropout layers. Dropout layers may be used during training of the neural network (e.g., to perform binary or multi-class classifications). The one or more dropout layers may randomly set certain weights as 0, which may set corresponding elements in the feature map as 0, so the neural network may avoid overfitting. The neural network may further comprise one or more dense layers, which comprise a fully connected network. In the dense layer, information may be passed through the fully connected network to generate a predicted classification of an object, and the error may be calculated. In some embodiments, the error may be backpropagated to improve the prediction. The one or more dense layers may comprise a Softmax activation function, which may convert a vector of numbers to a vector of probabilities. These probabilities may be subsequently used in classifications, such as classifications of signal (e.g., electrical currents and/or voltages) to the nucleoside monomer added to the polynucleotide.

The computer system 3700 illustrated in FIG. 9 may be understood as a logical apparatus that can read instructions from media 3711 and/or a network port 3705, which can optionally be connected to server 3709 having fixed media 3712. The system can include a CPU 3701, disk drives 3703, optional input devices such as keyboard 3715 and/or mouse 3713 and optional monitor 3707. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 3722.

FIG. 10 is a block diagram illustrating a first example architecture of a computer system that can be used in connection with example instances of the present disclosure. As depicted in FIG. 9, the example computer system can include a processor 3802 for processing instructions. Non-limiting examples of processors include: Intel Xeon™ processor, AMD Opteron™ processor, Samsung 32-bit RISC ARM 1176JZ(F)—S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 10, a high speed cache 3804 can be connected to, or incorporated in, the processor 3802 to provide a high speed memory for instructions or data that have been recently, or are frequently, used by processor 3802. The processor 3802 is connected to a north bridge 3806 by a processor bus 3808. The north bridge 3806 is connected to random access memory (RAM) 3810 by a memory bus 3812 and manages access to the RAM 3810 by the processor 3802. The north bridge 3806 is also connected to a south bridge 3814 by a chipset bus 3816. The south bridge 3814 is, in turn, connected to a peripheral bus 3818. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 3818. In some alternative architectures, the functionality of the north bridge can be incorporated into the processor instead of using a separate north bridge chip.

In some instances, a system 3800 can include an accelerator card 3822 attached to the peripheral bus 3818. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 3824 and can be loaded into RAM 3810 and/or cache 3804 for use by the processor. The system 3800 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example embodiments of the present disclosure.

In this example, system 3800 also includes network interface cards (NICs) 3820 and 3821 connected to the peripheral bus for providing network interfaces to external storage, such as Network Attached Storage (NAS) and other computer systems that can be used for distributed parallel processing.

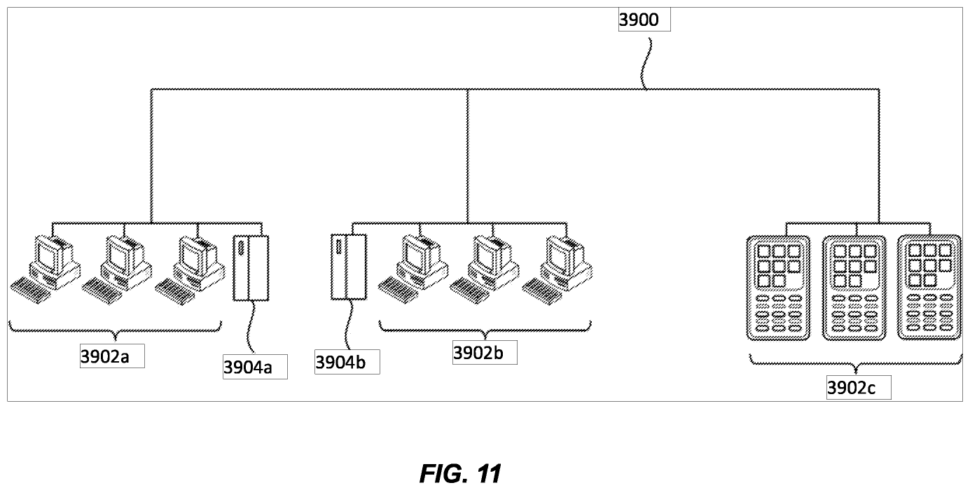

FIG. 11 is a diagram showing a network 3900 with a plurality of computer systems 3902a, and 3902b, a plurality of cell phones and personal data assistants 3902c, and Network Attached Storage (NAS) 3904a, and 3904b. In example embodiments, systems 3902a, 3902b, and 3902c can manage data storage and optimize data access for data stored in Network Attached Storage (NAS) 3904a and 3904b. A mathematical model can be used for the data and be evaluated using distributed parallel processing across computer systems 3902a, and 3902b, and cell phone and personal data assistant systems 3902c. Computer systems 3902a, and 3902b, and cell phone and personal data assistant systems 3902c can also provide parallel processing for adaptive data restructuring of the data stored in Network Attached Storage (NAS) 3904a and 3904b. FIG. 11 illustrates an example only, and a wide variety of other computer architectures and systems can be used in conjunction with the various embodiments of the present disclosure. For example, a blade server can be used to provide parallel processing. Processor blades can be connected through a back plane to provide parallel processing. Storage can also be connected to the back plane or as Network Attached Storage (NAS) through a separate network interface.

In some example embodiments, processors can maintain separate memory spaces and transmit data through network interfaces, back plane or other connectors for parallel processing by other processors. In other embodiments, some or all of the processors can use a shared virtual address memory space.

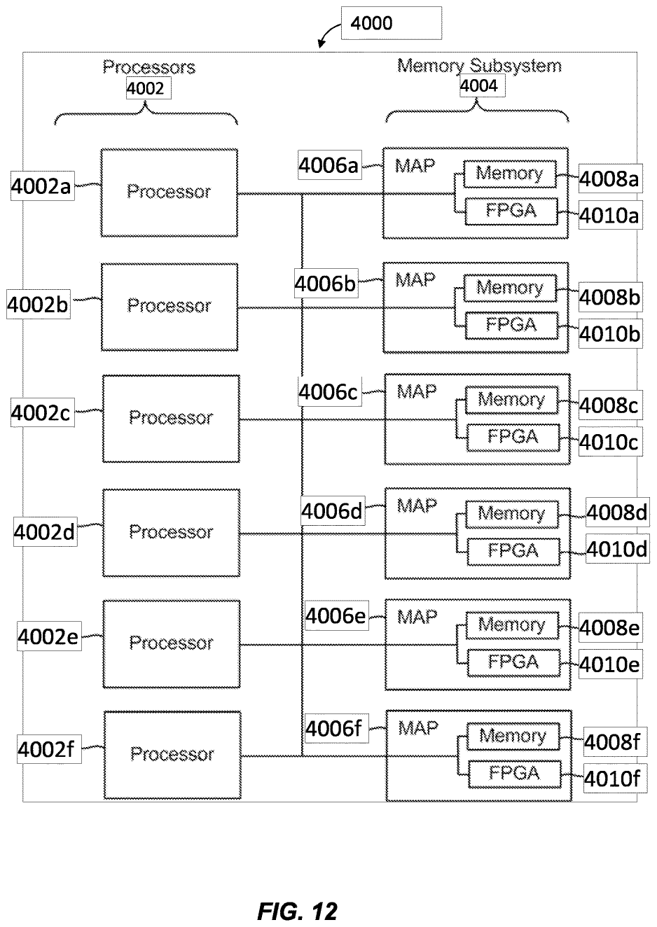

FIG. 12 is a block diagram of a multiprocessor computer system 4000 using a shared virtual address memory space in accordance with an example embodiment. The system includes a plurality of processors 4002a-f that can access a shared memory subsystem 4004. The system incorporates a plurality of programmable hardware memory algorithm processors (MAPs) 806a-f in the memory subsystem 4004. Each MAP 4006a-f can comprise a memory 4008a-f and one or more field programmable gate arrays (FPGAs) 4010a-f. The MAP provides a configurable functional unit and particular algorithms, or portions of algorithms can be provided to the FPGAs 4010a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 4008a-f, allowing it to execute tasks independently of, and asynchronously from, the respective microprocessor 4002a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example embodiments, including systems using any combination of general processors, co-processors, FPGAs and other programmable logic devices, system on chips (SOCs), application specific integrated circuits (ASICs), and other processing and logic elements. In some embodiments, all or part of the computer system can be implemented in software or hardware. Any variety of data storage media can be used in connection with example embodiments, including random access memory, hard drives, flash memory, tape drives, disk arrays, Network Attached Storage (NAS) and other local or distributed data storage devices and systems.

In example embodiments, the computer system can be implemented using software modules executing on any of the above or other computer architectures and systems. In other embodiments, the functions of the system can be implemented partially or completely in firmware, programmable logic devices such as field programmable gate arrays (FPGAs), system on chips (SOCs), application specific integrated circuits (ASICs), or other processing and logic elements. For example, the Set Processor and Optimizer can be implemented with hardware acceleration through the use of a hardware accelerator card.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1: Synthesis and Analysis of 15-Mer Oligomers with Deoxyinosine (dI) or Deoxyguanosine (dG)

Two 15-mer DNA oligomers were synthesized: A solution comprising a mixture of hydroquinone and benzoquinone was used as a redox solution for electrochemical deblocking of the protecting group, 4,4′-dimethoxytrityl (DMT). The mixture included 17 mM of TBA-HFP, 500 mM hydroquinone, 10 mM benzoquinone in acetonitrile.

A natural four nucleobase sequence, 5′-AGATCAGTCAGTGTCLLT (L stands for unylinker and is commercially available from Glen Research), was first synthesized using electrochemical deblocking conditions described herein, on an electrochemically enabled silicon chip. Acid was generated in an anodic process and in situ participates in DMT deprotection. Then DMT-removed oligos participate in subsequent regular coupling, capping, and oxidation process. The synthesis cycle described above is repeated until all nucleotides are assembled in desired sequence showed above. Then silicon chip is carried out to deprotection process under methylamine at 65° C. to remove protecting group on oligos as well as cleave the oligos from linker. After deprotection, the DNA was extracted using a 25% methanol solution (MeOH:H₂O=1:3) and a total volume of 200 uL was used to extract the DNA from the synthesis surface, which was subsequently dried down in SpeedVac.

A second 15-mer was synthesized under the same conditions, except that for replacement of dG with dI (5′-AIATCAITCAITITCLLT), and the synthesized oligomers were subsequently extracted following the same protocol.

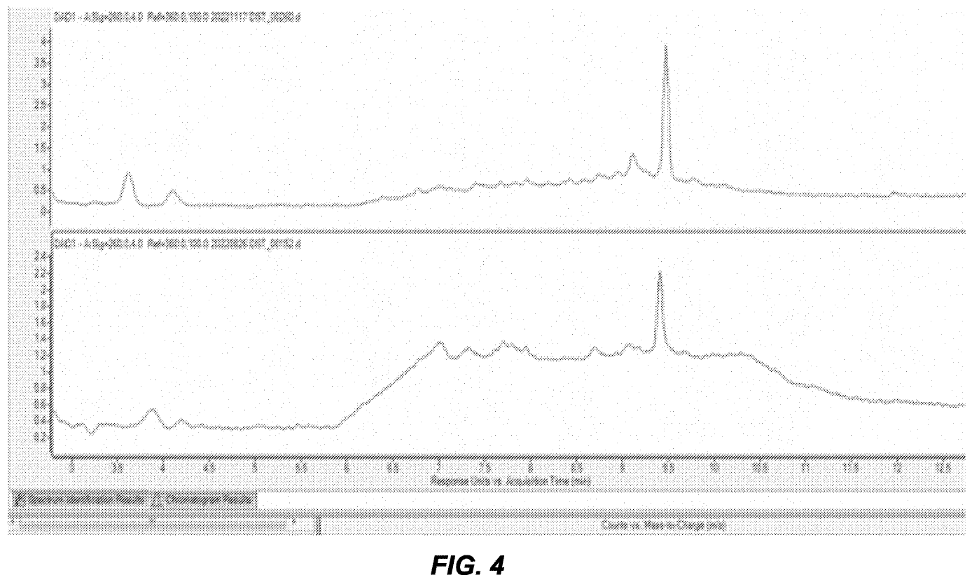

The two synthesized oligomers were collected and analyzed: The dried down samples were rehydrated with 13 uL of nuclease-free water, and aliquots were collected for analysis by a nanodrop spectrophotometer. The rest was dried down for analysis by LCMS The LCMS results are shown in FIG. 4. As shown, the second sequence with dI (top) resulted in twice the yield compared to the first sequence with dG (bottom).

### Example 2: Synthesis and Analysis of 90-Mer Oligomers with Deoxyinosine (dI) or Deoxyguanosine (dG)

Two 90-mer DNA oligomers are synthesized using the methods generally described in Example 1. A solution comprising a mixture of hydroquinone and benzoquinone is used as a redox solution for electrochemical deblocking of the protecting group, 4,4′-dimethoxytrityl (DMT).

The first sequence includes the four natural nucleobases, dA, dT, dC, and dG, while the second sequence replaces all dG with dI.

The two oligomers are synthesized, collected and analyzed.

### Example 3: Encoding an Item of Information in a Library with Inosine

An item of information is received by a computing system in the form of binary digits, 0 and 1. This first string is converted using a codec to a second string of symbols representing a plurality of polynucleotide sequences. The codec converts the first string to the second string using one or more rules, such as an error correction scheme, a codebook, a sequence constraint, or any combination thereof. The codebook maps the first string of binary digits 0 and 1 to a second string comprising letters for constructing nucleic acid sequences: A, T, C, and I. The sequence constraint can comprises one or more constraints related to length, inosine content, guanosine content, guanosine cytosine content, repeats of one or more bases, or any combination thereof. The codec is applied such that the second string of symbols representing a plurality of polynucleotides each comprise a data block storing a portion of the item of information, and a non-data block storing metadata. Metadata can comprise an index, data type, data size, data format, encryption codec, date of synthesis, date of last access, dates of previous handling, owner information, manufacture information, storage mechanism, or any combination thereof.

### Example 4: Increasing the Redox Resistance of a Nucleic Acid Sequence

A polynucleotide with sequences comprising a first string of symbols of nucleobases A, T, C, and G is received and sequenced using the systems and methods provided herein. A computing system comprising a program (e.g., codec) is used to determine which one or more bases in the polynucleotide to replace with a redox resistant base. In some examples, all of the G residues in the sequences polynucleotide are replaced with inosine to increase the redox resistance of the polynucleotide, thereby generating a second string of symbols of nucleobases A, T, C, and I. The new polynucleotide (with residues A, T, C and I) can be constructed and stored using the systems and methods generally described herein.

### Example 5: Synthesis of a Library with Mosine on a High Density Array Device

The polynucleotides with sequences comprising dI of any one Examples 1-4 are synthesized on a solid support comprising an addressable array of loci. The solid support can synthesize at least 10,000 polynucleotides with a length of 150 bases. The loci have a pitch distance of 10-200 nm, and the array comprises at least 1000 addressable loci. A material deposition system is provided that is in communication with the computing system that stores sequences of the polynucleotides to be synthesized.

The material deposition system comprises a substrate with the plurality of addressable loci for constructing the polynucleotides. The material deposition system further comprises a deposition unit for depositing one or more building blocks and reagents that are needed to construct the polynucleotides. The deposition unit deposits building blocks comprising nucleic acids monomers (A, T, C, or I) and reagents at select locations on the substrate for synthesis of the polynucleotides. The polynucleotides are extended from the surface of the substrate.

### Example 6: Storage of the Polynucleotides

Polynucleotides that are synthesized as generally illustrated in Example 5 are stored in a storage system, such as a data center.

In some examples, the substrate comprising the solid support as generally provided in Example 5 is integrated onto a rack unit, which is conveniently inserted or removed from a server rack. In some examples, the polynucleotides are removed from the solid support using the methods generally illustrated in Example 1, and stored in a compartment comprising a storage medium.

The rack can house a number of solid supports or compartments, with mechanical structures commonly used for mounting conventional computing and data storage resources in rack units. For example, a rack may comprise openings adapted to support disk drives, processing blades, and/or other computer equipment.

Polynucleotides (and the information stored in them) present on solid supports or in compartments can be accessed from the rack unit. Access includes removal of polynucleotides from solid supports or compartments, direct analysis of polynucleotides on the solid support or compartment, or any other method which allows the information stored in the polynucleotides to be manipulated or identified. Information in some instances is accessed using a robotic system. Information in some instances is accessed from a plurality of racks, a single rack, a single solid support in a rack, a portion of the solid support or compartment, or a single locus on a solid support.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present disclosure is further described by the following non-limiting items.
Item 1. A library comprising a plurality of polynucleotides, wherein the plurality of polynucleotides comprises a redox resistant base, and wherein the library encodes an item of information.
Item 2. The library of item 1, wherein at least 10% of bases in the plurality of polynucleotides comprise the redox resistant base.
Item 3. The library of items 1 or 2, wherein at least one of four canonical bases is replaced with a redox resistant base.
Item 4. The library of any one of items 1-3, wherein the redox resistant base is a non-canonical base.
Item 5. The library of item 4, wherein the non-canonical base can pair with a canonical base.
Item 6. The library of any one of items 1-5, wherein the redox resistant base has an oxidation potential larger than that of deoxyguanosine.
Item 7. The library of any one of items 4-6, wherein a ratio of redox resistant bases comprising the non-canonical base to canonical bases in the plurality of polynucleotides is about 1:1 to about 1:9.
Item 8. The library of any one of items 1-7, wherein the plurality of polynucleotides comprises at least one, two, or three different canonical bases.
Item 9. The library of any one of items 1-8, wherein the plurality of polynucleotides comprise adenosine, thymidine, cytidine, or any combination thereof.
Item 10. The library of item 4, wherein the non-canonical base comprises diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, or 2,6-diaminopurine.
Item 11. The library of item 4, wherein the non-canonical base comprises inosine.
Item 12. The library of any one of items 1-11, wherein the plurality ofpolynucleotides comprises 50 to 300 bases in length.
Item 13. The library of any one of items 1-12, wherein the item of information comprises text information, audio information, visual information, or any combination thereof.
Item 14. The library of any one of items 1-13, wherein each of the plurality of polynucleotides comprises at least one data block and at least one non-data block.
Item 15. The library of item 14, wherein the at least one data block comprises a portion of the item of information.
Item 16. The library of item 14, wherein the at least one non-data block comprises metadata related to the item of information.
Item 17. The library of item 16, wherein the metadata comprises an index, data type, data size, data format, encryption codec, date of synthesis, date of last access, dates of previous handling, owner information, manufacture information, storage mechanism, or any combination thereof.
Item 18. The library of any one of items 1-17, wherein the item of information is stored in the library with at least 10% redundancy.
Item 19. The library of any one of items 1-18, wherein the plurality of polynucleotides comprises 1000 to 500,000 polynucleotides.
Item 20. The library of any one of items 1-19, wherein the library comprises at least one adaptor sequence.
Item 21. The library of any one of items 1-20, wherein the at least one adaptor is configured to bind to a flow cell.
Item 22. The library of any one of items 1-21, wherein the library comprises at least one barcode.
Item 23. A method of storing an item of information in a plurality of polynucleotides, comprising:
   converting a first string of symbols encoding an item of information to a second string of symbols, wherein the second string of symbols comprises sequences of a plurality of polynucleotides in the library of any one of items 1-22.
Item 24. The method of item 23, wherein further comprising constructing the library comprising the plurality of polynucleotides.
Item 25. The method of item 23, wherein further storing the library comprising the plurality of polynucleotides.
Item 26. The method of item 23, wherein converting the item of information comprises:
   (a) generating a codec comprising one or more rules; and
   (b) applying the codec to the first string of symbols to generate the second string of symbols.
Item 27. The method of item 26, wherein the one or more rules comprises an error correction scheme, a codebook, a sequence constraint, or any combination thereof.
Item 28. The method of item 27, wherein the sequence constraint comprises one or more constraints related to length, inosine content, guanosine content, guanosine cytosine content, repeats of one or more bases, or any combination thereof.
Item 29. The method of any one of items 23-28, wherein the second string of symbols comprises at least one data block and at least one non-data block.
Item 30. The method of item 29, wherein at least one data block comprises a portion of the item of information.
Item 31. The method of item 29, wherein the at least one non-data block comprises metadata related to the item of information.
Item 32. The method of item 31, wherein the metadata comprises an index, data type, data size, data format, encryption codec, date of synthesis, date of last access, dates of previous handling, owner information, manufacture information, storage mechanism, or any combination thereof.
Item 33. The method of any one of items 23-32, wherein constructing comprises synthesizing the plurality of polynucleotides.
Item 34. The method of item 33, wherein synthesizing comprises electrochemical deblocking using electrochemical acid generation.
Item 35. The method of item 34, wherein electrochemical acid generation comprises contacting a protected polynucleotide with a composition comprising one or more redox compounds.
Item 36. The method of any one of items 23-35, wherein synthesizing comprises:
   (a) contacting a nucleoside attached to a solid support with a protected nucleoside, wherein the protected nucleoside is configured to form a covalent bond with the nucleoside to generate a protected polynucleotide;
   (b) contacting the protected polynucleotide with a composition comprising one or more redox compounds, and
   (c) applying a voltage to a solvent in fluid communication with the protected polynucleotide, wherein the voltage results in deprotection of the terminal nucleoside of the protected polynucleotide.
Item 37. The method of item 35 or 36, wherein the composition further comprises an organic salt and at least one solvent.
Item 38. The method of any one of items 35-37, wherein the one or more redox compounds comprises a substituted or unsubstituted quinone.
Item 39. The method of any one of items 35-38, wherein the one or more redox compounds comprises a mixture of quinone and benzoquinone.
Item 40. The method of item 37, wherein the organic salt comprises a tetraalkylammonium cation.
Item 41. The method of item 37, wherein the organic salt comprises a hexafluorophosphate anion.
Item 42. The method of item 37, wherein the organic salt is tetrabutylammonium hexafluorophosphate.
Item 43. The method of any one of items 37-42, wherein the at least one solvent is acetonitrile, methanol, ethanol, dichloromethane, chloroform, 1,2-dichloromethane, dimethylformamide, ethylene glycol, propylene carbonate, or a mixture thereof.
Item 44. The method of any one of items 35-43, wherein a concentration of the one or more redox compounds is 0.1-2M.
Item 45. The method of any one of items 37-44, wherein concentration of the organic salt is 10-50 mM.
Item 46. The method of any one of items 36-45, wherein the voltage is less than 2 volts.
Item 47. The method of any one of items 36-46, wherein the voltage is 0.1-2 volts.
Item 48. The method of any one of items 36-47, wherein the voltage is applied for 0.001-5000 seconds.
Item 49. The method of any one of items 36-48, wherein the voltage is applied for 0.001-5 seconds.
Item 50. The method of any one of items 36-49, wherein the voltage is applied in one or more pulses.
Item 51. The method of any one of items 36-50, wherein the time between pulses is 0-500 milliseconds.
Item 52. The method of any one of items 35-51, wherein the protected polynucleotide comprises an acid-cleavable protecting group.
Item 53. The method of any one of items 36-52, wherein the voltage generates acid.
Item 54. The method of any one of items 23-53, further comprising retrieving the item of information.
Item 55. The method of item 54, wherein retrieving the item of information comprises:
   (a) sequencing the library comprising the plurality of polynucleotides to obtain a readout; and
   (b) converting readout into the item of information.
Item 56. The method of item 54 or 55, wherein retrieving the item of information further comprises amplifying the plurality of polynucleotides.
Item 57. The method of any one of items 23-56, wherein the item of information is retrieved with at least 99% accuracy.
Item 58. The method of any one of items 55-57, wherein converting the readout into the item of information comprises:
   (a) applying the codec or a portion thereof to the readout comprising a third string of symbols to generate a fourth string of symbols; and
   (b) assembling the fourth string of symbols to retrieve the item of information.
Item 59. The method of item 58, wherein the second string of symbols and the third string of symbols comprise nucleic acid sequences.
Item 60. The method of item 58, wherein the second string of symbols and the third string of symbols are at least 99% identical.
Item 61. The method of item 58, wherein the first string of symbols and the fourth string of symbols are at least 99% identical.
Item 62. A method for increasing fidelity of DNA encoding an item of information, comprising replacing one or more bases of a DNA sequence with a redox resistant base.
Item 63. A method for storing DNA encoding an item of information, comprising:
   (a) receiving a DNA sequence encoding an item of information;
   (b) replacing one or more bases of the DNA sequence with a redox resistant base; and
   (c) storing the DNA sequence with the redox resistant base.
Item 64. The method of item 62 or 63, wherein replacing the one or more bases comprises replacing at least 10% of bases in the DNA sequence with the lowest oxidation potential.
Item 65. A device for polynucleotide synthesis comprising:
   a surface comprising a plurality of loci configured for polynucleotide synthesis of the library of any one of items 1-22; and
   a plurality of vias or routing configured for addressable control of the plurality of loci,
   wherein the area of each loci is 50-500 nm.
Item 66. The device of item 65, wherein the loci comprises a pitch distance of no more than 1000 nm.
Item 67. The device of item 65 or 66, wherein the device comprises at least 10 loci per square micron.
Item 68. The device of any one of items 65-67, wherein the device is integrated into a CMOS.
Item 69. The device of any one of items 65-68, wherein the device further comprises a fluidics interface.
Item 70. A device for storing an item of information in a plurality of polynucleotides, comprising:
   (a) one or more compartments, wherein each compartment comprises:
      (i) a library of any one of items 1-22 or a portion thereof, wherein the library encodes the item of information, or a portion thereof; and
      (ii) a medium for storing the library or the portion thereof.
Item 71. The device of item 70, wherein the one or more compartments are in communication.
Item 72. The device of item 70, wherein the one or more compartments are not in communication.
Item 73. The device of any one of items 70-72, wherein the one or more compartments are independently accessible.
Item 74. The device of any one of items 70-73, wherein each of the one or more compartments are independently accessible via a robotic system.
Item 75. The device of any one of items 70-74, wherein the medium comprises a solid, a liquid, a gas, or any combination thereof.
Item 76. The device of any one of items 70-75, wherein a medium comprises a salt solution at a molar ratio of less than 20:1 salt cation to phosphate groups in the DNA.
Item 77. The method of item 76, wherein the salt solution is dried to create a dried product.
Item 78. The device of any one of items 70-77, further comprising a solid support comprising a surface.
Item 79. The device of item 78, further comprising a plurality of structures located on the surface, wherein the plurality of polynucleotide are extended from the plurality of structures.
Item 80. A system for storing an item of information, comprising:
   (a) a computing system comprising at least one processor and instructions executable by the at least one processor to perform one or more operations, the one or more operations comprising: converting a first string of symbols to a second string of symbols, wherein the second string of symbols comprises a DNA sequence with a redox resistant base; and
   (b) a material deposition system in communication with the computing system, comprising:
      (iii) a substrate for constructing the DNA sequence; and
      (iv) a deposition unit for depositing one or more building blocks, reagents, or both for constructing the DNA sequence.

What is claimed is:

1. A library comprising a plurality of polynucleotides, wherein the plurality of polynucleotides comprises a redox resistant base, and wherein the library encodes an item of information.

2. The library of claim 1, wherein at least 10% of bases in the plurality of polynucleotides comprise the redox resistant base.

3. The library of claim 1 or 2, wherein at least one of four canonical bases is replaced with a redox resistant base.

4. The library of any one of claims 1-3, wherein the redox resistant base is a non-canonical base.

5. The library of claim 4, wherein the non-canonical base can pair with a canonical base.

6. The library of any one of claims 1-5, wherein the redox resistant base has an oxidation potential larger than that of deoxyguanosine.

7. The library of any one of claims 4-6, wherein a ratio of redox resistant bases comprising the non-canonical base to canonical bases in the plurality of polynucleotides is about 1:1 to about 1:9.

8. The library of any one of claims 1-7, wherein the plurality of polynucleotides comprises at least one, two, or three different canonical bases.

9. The library of any one of claims 1-8, wherein the plurality of polynucleotides comprise adenosine, thymidine, cytidine, or any combination thereof.

10. The library of claim 4, wherein the non-canonical base comprises diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, or 2,6-diaminopurine.

11. The library of claim 4, wherein the non-canonical base comprises inosine.

12. The library of any one of claims 1-11, wherein the plurality of polynucleotides comprises 50 to 300 bases in length.

13. The library of any one of claims 1-12, wherein the item of information comprises text information, audio information, visual information, or any combination thereof.

14. The library of any one of claims 1-13, wherein each of the plurality of polynucleotides comprises at least one data block and at least one non-data block.

15. The library of claim 14, wherein the at least one data block comprises a portion of the item of information.

16. The library of claim 14, wherein the at least one non-data block comprises metadata related to the item of information.

17. The library of claim 16, wherein the metadata comprises an index, data type, data size, data format, encryption codec, date of synthesis, date of last access, dates of previous handling, owner information, manufacture information, storage mechanism, or any combination thereof.

18. The library of any one of claims 1-17, wherein the item of information is stored in the library with at least 10% redundancy.

19. The library of any one of claims 1-18, wherein the plurality of polynucleotides comprises 1000 to 500,000 polynucleotides.

20. The library of any one of claims 1-19, wherein the library comprises at least one adaptor sequence.
